(19) United States
(12) Patent Application Publication
(10) Pub. No.: US 2025/0382676 A1
(43) Pub. Date: Dec. 18, 2025

(54) METHODS OF IDENTIFYING STRAINS ASSOCIATED WITH THE HUMAN FEMALE GENITOURINARY TRACT

(71) Applicant: Freya Biosciences ApS, Copenhagen Ø (DK)

(72) Inventors: Elleke Fenna BOSMA, Copenhagen (DK); Colleen Denise ACOSTA, Copenhagen (DK); Johan E.T. VAN HYLCKAMA VLIEG, Copenhagen (DK); Thomas Gundelund RASMUSSEN, Copenhagen (DK); Brynjulf MORTENSEN, Copenhagen (DK)

(21) Appl. No.: 18/863,199

(22) PCT Filed: May 11, 2023
(86) PCT No.: PCT/IB2023/054900

§ 371 Date: Nov. 5, 2024

Related U.S. Application Data
(60) Provisional application No. 63/340,887, filed on May 11, 2022.

(51) Int. Cl. C12Q 1/689; A61K 35/747

(52) U.S. Cl. CPC C12Q 1/689; A61K 35/747; C12Q 2600/156; C12Q 2600/172

(57) ABSTRACT

Provided are methods of identifying bacterial strains capable of colonizing the human female genitourinary tract and compositions thereof, as well as methods of defining vaginal microbial communities and profiles.

### RELATED APPLICATIONS

This application is a U.S. National Stage Application of International Application No. PCT/IB2023/054900 filed on May 11, 2023, which claims priority to U.S. Provisional Patent Application No. 63/340,887, filed on May 11, 2022, each of which are incorporated herein by reference in their entireties.

### BACKGROUND OF THE INVENTION

The vagina is a fibromuscular tubular tract leading from the uterus to the exterior of the body in females. A healthy vagina is colonized by a mutually symbiotic flora of microorganisms, in particular of the genus Lactobacillus, that protects its host from vaginal infections. The acidity of a healthy vagina of a woman of child-bearing age (pH about 3.8-4.5) is thought to be due to the degradation of secreted glycogen/glucose to lactic acid and acetate by lactobacilli. Acidic conditions are unfavorable for the growth of many pathogenic microorganisms and pathobionts, including bacteria, protozoa and viruses. However, an imbalance in the vaginal microbiota may result in overgrowth of pathogenic microorganisms and pathobionts, resulting in dysbiosis, inflammation and/or infections.

Lactobacillus-containing products (comprising, e.g., Lactobacillus acidophilus, Lactobacillus rhammosus, or Lactobacillus gasseri) for intravaginal or oral use have been available for many years. These products include vaginal suppositories containing lyophilized Lactobacillus acidophilus or other Lactobacillus species (e.g., Lactobacillus rhamnosus, or Lactobacillus gasseri) of human origin as well as various nutritional supplements. These products have been largely non-efficacious due to the products' inability to colonize the vagina with the exogenous lactobacilli. This might be due to poor quality or the use of ecologically unsuitable strains.

The colonization and establishment of isolated bacteria, such as single strains, in the vaginal microbial niche is difficult to predict from in vitro experiments, e.g., it is common for different bacterial species in the vaginal microbial niche to interact, and these interactions are difficult to recreate in the laboratory. It is well established and broadly accepted that different women can be colonized by different compositions of species/strains of lactobacilli and that this composition of lactobacilli changes over time. Thus, even if bacterial species display promising in vitro results, it might not colonize or engraft in vivo and provide the desired therapeutic effect, or the effect is transient as the strains(s) delivered are not able to collectively adapt to provide a maintained colonization over time. For example, a specified value for adherence to the vaginal mucosa (vaginal epithelial cells—VEC), ability to produce anti-microbial/anti-pathogenic substances, such as, e.g., hydrogen peroxide and/or bacteriocins, ability to produce lactic acid for acidification, and fast doubling time, in culture are often measured in vitro, though these characteristics have not routinely led to successful predictions of the ability of specific strains to engraft in the vaginal niche (their ability to colonize the vagina) in vivo.

Thus, a need exists for the identification of bacterial taxa capable of colonizing the human female genitourinary tract and a better understanding of the in vivo environment of the genitourinary tract and overall health profile of subjects receiving such strains.

### SUMMARY OF THE INVENTION

Provided herein are methods for identifying one or more bacterial strains capable of colonizing the human female genitourinary tract, e.g., of a female recipient that exhibits, e.g., a dysbiosis of the genitourinary tract.

Further provided herein are methods of defining vaginal microbial communities, such as donor and/or recipient vaginal microbial communities. Defining the vaginal microbial communities can be performed, e.g., to increase the success rate of colonizing the recipient's female genitourinary tract with one or more strains capable of colonizing the human female genitourinary tract identified by the methods described herein.

The first aspect of the invention relates to methods of identifying one or more bacterial strains capable of colonizing the female genitourinary tract comprising providing a donor sample comprising a substantially complete vaginal microbiota preparation (SCVMP) derived from a donor female comprising a plurality of bacterial strains; administering an effective amount of the donor sample to a recipient female's genitourinary tract that exhibits a dysbiotic vaginal microbial niche; assessing a desired change in dysbiosis of the recipient female's genitourinary tract over a predetermined time period; identifying one or more bacterial strains of the donor sample that colonize the vaginal microbial niche of the recipient female's genitourinary tract by analyzing a recipient sample comprising a plurality of bacterial strains obtained from the recipient female's genitourinary tract after achieving the desired change in dysbiosis

The second aspect of the invention relates to a composition (e.g., microbial composition) or pharmaceutical composition comprising one or more (isolated) bacterial strains capable of colonizing and engrafting the female genitourinary tract obtainable by the methods described herein.

The third aspect of the invention relates to a dosage form comprising the (microbial) composition or pharmaceutical composition of the invention, wherein said dosage form is formulated for vaginal administration.

A fourth aspect of the invention relates to a method of defining one or more vaginal microbial donor communities, the method comprising:
A. obtaining a plurality of vaginal donor samples, wherein each donor sample comprises vaginal microbes obtained from a different non-dysbiotic (e.g., healthy) donor female (e.g., from vaginal fluid, e.g., a cervicovaginal secretion),
B. using an analytical technique to obtain a plurality of vaginal microbial profiles from the plurality of vaginal microbial samples,
C. identifying a profile (e.g., a pangenome or supragenome profile) based on the plurality of microbial profiles, thereby defining a plurality of vaginal microbial donor communities,
D. optionally, wherein the profile is selected at least in part based on identifying at least one genetic element (or a plurality of genetic elements) associated with the capability to successfully engraft/colonize the female genitourinary tract of a female recipient, and
providing the profile to a user. In a further aspect, the invention relates to a composition or pharmaceutical composition comprising one or more (isolated) bacterial strains capable of colonizing and engrafting the vaginal microbial niche identified according to the methods described herein.

In a further aspect, the invention provides a (microbial) composition or pharmaceutical composition comprising one or more (isolated) bacterial strains which form a vaginal microbial donor community as defined herein.

The method may further comprise (a) nucleic acid sequencing of the microbial constituents of the recipient sample after determining a desired change in dysbiosis, and of the donor sample, (b) comparing the sequencing results for the microbial constituents of the recipient sample with the microbial constituents of the donor sample to determine sequence identity of strains residing in both, and (c) determining that one or more strains is capable of colonization if the one or more strains identified from the recipient sample match one or more strains from the donor sample by a predetermined degree of sequence identity. The nucleic acid sequencing may be whole genome sequencing. The nucleic acid sequencing may be haplotype analysis, e.g. haplotype analysis of single copy core genes identified from metagenome assembled genomes (MAGs), wherein single nucleotide polymorphisms (SNPs) may be used to define the haplotypes. The sequence identity of a strain or genetic element thereof is at least 98%, 99%. 99.5%, 99.8%, or 99.9%. The one or more strains of the genus Lactobacillus can be identified as capable of colonizing. The one or more strains of the genus Lactobacillus can be identified (e.g., detectable by sequencing) in the donor sample and in the recipient sample after administration of the donor sample to the recipient female but not prior thereto. The one or more strains of the genus Lactobacillus can be identified (e.g., detectable by sequencing) in the recipient sample after a desired change in dysbiosis of the recipient female's genitourinary tract has occurred. The one or more strains of the genus Lactobacillus can be identified (e.g., detectable by sequencing) in the recipient sample after one (two, three, four or five) menstruation cycle(s) post administration of the donor sample. The one or more strains of the genus Lactobacillus can be identified (e.g., detectable by sequencing) in the recipient sample after one (two, three, four, five, six, or 12) month(s) post administration of the donor sample. The donor sample comprising a substantially complete vaginal microbiota preparation may be obtained by a method comprising one or more of the following steps: a) diluting the sample in a buffer or diluent to obtain a predetermined viscosity suitable for administration to a female recipient, b) pooling the sample from the same donor (or from different donors) to obtain a sample of predetermined quantity (e.g., of CFUs contained in the sample), and/or c) filtering the sample (e.g., to remove cell or bacterial aggregates or other unwanted matter residing in the cervicovaginal secretion of the donor sample). The buffer or diluent can be saline. The method may not include culturing the donor sample comprising the substantially complete vaginal microbiota preparation and isolating strains prior to administering the donor sample to the recipient female. The method may not include testing the strains comprised in the donor sample prior to administering the donor sample to the recipient female for one or more of: a) their ability to adhere to epithelial cells in vitro (e.g., obtaining a vaginal epithelial cell (VEC) cohesion value), b) their ability to produce hydrogen peroxide, c) their ability to produce lactic acid in vitro, d) their growth rate in vitro, e) their ability to kill or inhibit pathogens in vitro, or f) their viability after lyophilization. The plurality of bacterial strains may comprise one or more Lactobacillus strains. The plurality of bacterial strains may consist of between 1 and 30 (2 and 30, 2 and 20, 3 and 25, 3 and 15) lactic acid producing, culturable and in vitro propagatable bacterial strains obtained from the donor sample comprising the substantially complete vaginal microbiota preparation. The recipient female may be clinically diagnosed with bacterial vaginosis. The change in dysbiosis of the recipient female's genitourinary tract may be assessed within one, two, or three, six, twelve week(s), 6 months, 9 months, 12 months, 18 months or within 24 months post administration of the donor sample. The change in dysbiosis of the recipient female's genitourinary tract can be assessed within 1, 2, 3, 4, 5, 6 or 7 day(s) post administration of the donor sample. The desired change in dysbiosis of the recipient female's genitourinary tract may be detectable within one, two, or three week(s) post administration of the donor sample. The desired change in dysbiosis of the recipient female's genitourinary tract may be detectable within two, three, six, nine, or twelve months post administration of the donor sample. The desired change in dysbiosis of the recipient female's genitourinary tract may be detectable within 1, 2, 3, 4, 5, 6 or 7 day(s) post administration of the donor sample. The desired change may be a drop in vaginal pH, e.g., by at least pH 0.3, 0.5, 1.0 or at least pH 1.5. Any one of the steps may be repeated one or more (e.g., two, three, or four) times. A donor sample from the same donor may be administered to the same recipient twice. A donor sample from the same donor may be administered to the same recipient three times. The recipient may receive two donor samples from different donors. The recipient may receive three donor samples from at least two different donors. The recipient may receive (i.e., is administered) another donor sample within 1, 2, 3, 4, 5, 6 or 7 days of the first donor sample. The recipient receives (i.e., is administered) another donor sample within 1, 2, 3, 4, 5, 6, 8, 10, or 12 weeks of the first donor sample. The method may further comprise isolating (and optionally propagating) the one or more identified bacterial strains that colonize the microbial niche of the recipient female's genitourinary tract. The method may further comprise testing the isolated strains for one or more of: a) low abundance of antibiotic resistance markers (AMR), b) pathogenicity or presence of nucleic acids encoding pathogenicity factors, c) lysogenic phages, d) process relevant traits such as yield during fermentation, viability after lyophilization, or stability during storage, (e) immune modulatory properties and f) ability to colonize a dysbiotic female recipient. The method may further comprise determining that the donor sample is substantially free of pathogens and pathobionts prior to administration to the recipient. The method may further comprise determining that the donor sample is substantially free of Gardnerella spp., Atopobium spp., and Prevotella spp. prior to administration to the recipient. The method may further comprise determining that the donor sample is substantially free of antimicrobial resistance (AMR) genes prior to administration to the recipient. The method may further comprise determining that the donor sample is substantially free of human sperm (spermatozoa) prior to administration to the recipient. The method may further comprise determining that the female donor is substantially free of any one or more (two or more, three or more, or four or more) of: (i) bacteria involved in bacterial vaginosis (e.g., Gardnerella spp. and Mobiluncus spp.), (ii) yeast (e.g., Candida, Cryptococcus, and Saccharomyces species), (iii) sexually transmitted pathogens (including Neisseria gonorrhea, Chlamydia trachomatis, and Trichomonas vaginalis), (iv) bacteria involved in urinary tract infections (e.g., Escherichia coli, Staphylococcus, Chlamydia, and Mycoplasma), and (v) viruses (e.g., HIV, human papilloma virus (HPV), hepatitis B virus, hepatitis C virus, HSV-2).

The female donor may comprise a healthy (e.g., non-dysbiotic) microbiome of the genitourinary tract (e.g., vaginal microbial niche). The donor sample may comprise about 80-99.9% Lactobacillus crispatus. The donor sample may comprise about 80-99.9% Lactobacillus iners. The donor sample may comprise about 80-99.9% Lactobacillus jensenii. The donor sample may comprise about 80-99.9% Lactobacillus gasseri. The donor sample may comprise about 80-99.9% of one, two, three, or four of Lactobacillus crispatus, Lactobacillus iners, Lactobacilhis jensenii, and Lactobacillus gasseri. The donor sample may be formulated as a pharmaceutical composition (e.g., comprising a pharmaceutically acceptable buffer or diluent) for administration to the genitourinary tract of a recipient female.

The recipient female may further receive an agent, e.g., an antibiotic (e.g., metronidazole), and anti-fungal, an immunological agent, an acidifying agent (e.g., lactic acid), a prebiotic, or a hormonal agent (e.g., estrogen). The agent may be administered to the recipient female before, concurrent with, and/or after administration of the donor sample. The agent may be administered 1, 2, 3, 4, 5, 6, 7, 10 or 14 days prior to administration. The agent may be administered up to 1, 2, 3, or up to 4, weeks after administration.

In another aspect, the invention relates to a method of defining one or more vaginal microbial donor communities, the method comprising: obtaining a plurality of vaginal donor samples, wherein each donor sample comprises vaginal microbes obtained from a different non-dysbiotic (e.g., healthy) donor female (e.g., from vaginal fluid, e.g., a cervicovaginal secretion), using an analytical technique to obtain a plurality of vaginal microbial profiles from the plurality of vaginal samples, identifying profiles from among the plurality of microbial profiles, thereby defining a plurality of vaginal microbial donor communities, wherein the profiles comprise one or more strains belonging to one or more species selected from Lactobacillus crispatus, Lactobacillus iners, Lactobacillus jensenii, and Lactobacillus gasseri, wherein the one or more strains are capable of colonizing the female genitourinary tract of a female recipient of the vaginal microbial donor community, and providing the profiles to a user. The microbial profiles can be obtained by preparing at least one nucleic acid sample from at least one species of microbiota present in the vaginal donor sample comprising a substantially complete vaginal microbiota preparation. The nucleic acid may be prepared directly from the vaginal donor sample without isolating and/or propagating strains from the sample (e.g., in vitro). The analytical technique may be whole genome sequencing. The method may comprise determining which microbes are present in the single samples and selecting vaginal microbial donor communities from the profiles that comprise at least 80% lactic acid producing bacteria, e.g., for administration to a recipient female. The method may comprise selecting vaginal microbial donor communities from the profiles that comprise about 80-99.9% Lactobacillus crispatus, e.g., for administration to a recipient female. The method may comprise selecting vaginal microbial donor communities from the profiles that comprise about 80-99.9% Lactobacillus iners, e.g., for administration to a recipient female. The method may comprise selecting vaginal microbial donor communities from the profiles that comprise about 80-99.9% Lactobacillus jensenii, e.g., for administration to a recipient female. The method may comprise selecting vaginal microbial donor communities from the profiles that comprise about 80-99.9% Lactobacillus gasseri, e.g., for administration to a recipient female. The method may comprise selecting vaginal microbial donor communities from profiles that comprise of one, two, three, or four of Lactobacillus crispatus, Lactobacillus iners, Lactobacillus jensenii, and Lactobacillus gasseri, e.g., for administration to a recipient female. In some embodiments, the vaginal microbial donor communities may further comprise other lactobacilli, including Lactobacillus amylovorus, Lactobacillus gallinarum, Lactobaillus helveticus, or Limosilactobacillus vaginalis.

The recipient female may exhibit dysbiosis (e.g., exhibits a dysbiotic vaginal microbial niche). In some embodiments, the recipient female may exhibit bacterial vaginosis (BV).

The vaginal donor samples can be obtained from a female donor who is substantially free of any one or more (two or more, three or more, or four or more) of: (i) bacteria involved in bacterial vaginosis (e.g., Gardnerella and Mobiluncus), (ii) yeast (e.g., Candida, Cryptococcus, and Saccharomyces species), (iii) sexually transmitted pathogens (including Neisseria gonorrhea, Chlamydia trachomatis, and Trichomonas vaginalis), (iv) bacteria involved in urinary tract infections (e.g., Escherichia coli, Streptococcus, Staphylococcus, Chlamydia, and Mycoplasma), and (v) viruses (e.g., HIV, human papilloma virus (HPV), hepatitis B virus, hepatitis C virus, HSV-2). The vaginal donor samples can be obtained from a female donor comprising a healthy (e.g., non-dysbiotic) microbiome of the genitourinary tract (e.g., vaginal microbial niche).

The user may obtain the profiles for the purpose of determining a suitable donor for administration of the vaginal microbial donor communities to a recipient in need thereof.

The donor and the recipient may be human.

### BRIEF DESCRIPTION OF THE DRAWINGS

FIG. 1 is a schematic representation of the donor program to produce donor samples comprising substantially complete vaginal microbiota preparations. Donors were selected based on microbiome sequencing, a medical examination, and the absence of certain diseases. Testing was performed both before and after the donation visits. All samples provided by the donor were subjected to quality control (see, e.g., FIG. 5). Samples were released only when donor and samples passed all requirements.

FIG. 2 shows the classification of vaginal microbiomes from a cohort of 96 female subjects based on taxonomic composition as assessed by shotgun DNA sequencing to identify suitable donors from which to obtain cervicovaginal secretions. Relative abundance of bacteria was measured, and subjects were classified as “healthy”, “dysbiotic” and “undefined” using the classification parameters indicated.

FIG. 3 shows stacked bar graphs of relative abundance of bacterial taxa (genera and species) in the donors from the cohort of 96 female subjects (see, FIG. 2) with a healthy vaginal microbiome (n=61), as assessed by shotgun sequencing. All donor microbiomes in this graph contain at least 80% of vaginal Lactobacillus species (Lactobacillus crispatus, Lactobacillus gasseri, Lactobacillus jensenii, and Lactobacillus iners) or mixtures thereof, and less than 5% selected pathogens (Atopobium spp., Prevotella spp., B. vaginale, and F. vaginae) or mixtures thereof.

FIG. 4 shows stacked bar graphs of relative abundance of bacterial taxa (genera and species) in the donors from the cohort of 96 female subjects (see, FIG. 2) with a dysbiotic vaginal microbiome (left, n=27) or undefined microbiome (right, n=8) as assessed by shotgun sequencing. Dysbiotic microbiomes contain at least 20% species from selected vaginal pathogens (Atopobium spp., Prevotella spp., B. vaginale, and F. vaginae) or mixtures thereof, and less than 10% of vaginal Lactobacillus species (Lactobacillus crispatus, Lactobacillus gasseri, Lactobacillus jensenii, and Lactobacillus iners) or mixtures thereof.

FIG. 5 shows a flowchart depicting an exemplary procedure to obtain cervicovaginal secretions and processing thereof. FIG. 5A provides a general overview, whereas FIG. 5B provides a concrete exemplary embodiment of the procedure. The secretions were split into samples for characterization and quality control (including, e.g., viability count, DNA sequencing and pathogen screening) and processing into samples comprising a substantially complete vaginal microbiota preparation.

FIG. 6 shows stacked bar graphs of four main Lactobacillus species present in donor sample comprising the substantially complete vaginal microbiota preparations from 9 donors (FIGS. 6A-6I) as assessed by shotgun sequencing. The results from eight representative donation visits are shown for each donor. Where present, the vertical line indicates a new round of visits as recurring donor.

FIG. 7 shows plots depicting stability of donor samples comprising substantially complete vaginal microbiota preparations after storage in −80° C. for up to 11 months. Each data point represents one sample; each connecting line indicates samples from one donation visit of one donor. Similar symbols and shading indicate similar microbiomes.

FIGS. 8A and 8B shows bar graphs of viability of donor samples comprising substantially complete vaginal microbiota preparations after thawing from −80° C. A. Gradual thawing of the samples and warming them up until approx. 30° C. B. Effect of repeated freeze-thaw cycles or prolonged incubation at ambient temperature (RT=room temperature) on cell viability.

FIGS. 9A and 9B shows stacked bar graphs of relative bacterial abundance on a species level of a substantially complete vaginal microbiota preparations in two recipients and their respective donors, as measured by shotgun sequencing. All recipients were enrolled based on their microbiome at screening; a baseline microbiome sample was taken just prior to dosing. The asterisk (*) indicates that one menstruation took place before that sample was taken. Two asterisks (**) indicate that two menstrual cycles passed.

FIG. 10 shows a principal component analysis (PCA) plot for two recipients of a donor sample comprising a substantially complete vaginal microbiota preparation, pre- and post-dosing, and their respective donors. The variables used as input are the relative abundances of bacterial species as measured by shotgun sequencing. The same input was used as for the graphs in FIG. 9.

FIGS. 11A and 11B shows a heatmap of L. crispatus haplotypes identified in donor samples (FIG. 11A) and recipient samples (FIG. 11B). Haplotypes are indicated in columns; samples obtained during separate visits are indicated in rows. Samples that lacked any L. crispatus are not shown. FIG. 11A. L. crispatus haplotypes found in donor samples comprising substantially complete vaginal microbiota (SCVMP) preparations from multiple donations (each row represents one donation from a single visit) and different donors. FIG. 11B. L. crispatus haplotypes found in two recipients who received treatment with donor samples comprising a SCVMP derived from different visits of donor 1 (see FIG. 11A.).

FIG. 12 shows a simplified schematic of the development of strains using the in-human-first approach, where only engrafting strains after treatment with a substantially complete vaginal microbiota preparation are further tested as candidate strains for further clinical development.

### DETAILED DESCRIPTION

### Methods to Identify Bacterial Strains Capable of Colonizing and Engrafting the Female Genitourinary Tract

Provided herein are methods of identifying one or more bacterial strains capable of colonizing the human female genitourinary tract, e.g., of a female recipient that exhibits, e.g., a dysbiosis of the genitourinary tract. The genitourinary tract includes the reproductive tract, and it is thought that a link exists between the vaginal and endometrial spaces (e.g., that they represent interconnected biogeographical niches) and the exchange of e.g., microorganisms and immune molecules between the niches (Chen C et al., The microbiota continuum along the female reproductive tract and its relation to uterine-related diseases, Nature Communications vol 8, 875 (2017)).

Preparations of lactic acid producing bacteria (e.g., including lactobacilli) known in the art generally are not sufficiently capable of colonizing and engrafting in vivo, e.g., in the urogenital tract of a human female recipient of the preparation, e.g., a female exhibiting dysbiosis of the vaginal microbial niche. Among others, three main issues have hampered the discovery of lactic acid producing bacteria (e.g., including lactobacilli) capable of colonizing and engrafting in vivo: (1) the source of the bacteria, (2) the process of identifying and isolating suitable bacteria, (3) the route of administration of the bacteria, and (4) the combination of the plurality of bacteria, e.g. the specific combination of bacterial strains.

The inventors realized that to increase chances of obtaining strains of lactic acid producing bacteria (e.g., including lactobacilli) that are capable of colonizing and engrafting the female reproductive tract in vivo the following conditions are favorable:

The preparations of lactic acid producing bacteria (e.g., including lactobacilli) are sourced from a human female donor exhibiting healthy (non-dysbiotic) vaginal microbiota; i.e., the lactic acid producing bacteria that will be administered to a human female recipient are derived from a microbial niche (the donor female's urogenital tract microbiota) that is adapted to survive and thrive in this environment; the lactic acid producing bacteria are not sourced from food or other animal sources. Provided herein are methods that include bacterial preparations derived from healthy human female donors.

Strains capable of colonizing and engrafting in vivo in human subjects are identified after the donor preparation (comprising the lactic acid producing bacteria) has been administered to a female recipient, e.g., after a predetermined period of time, not prior to the administration to a recipient which is typically described in the art. Generally, the art describes methods that include first taking a (vaginal) sample from a healthy individual, then isolating and propagating individual strains in vitro (in culture), followed by in vitro testing to assess the ability to colonize (e.g., adherence testing to vaginal epithelia cells (VEC) or HeLa cells, and to produce anti-microbial agents, e.g., hydrogen peroxide) in vitro, and finally administering a strain(s) to a recipient. This has the disadvantage that any in vitro testing comprises a reductionist simulation of the in vivo environment and results have been mixed as to the extent of predictability of success, using such in vitro methods. Provided herein are methods that include engrafting bacterial preparations derived from healthy human female donors in recipient human females (e.g., females who exhibit a dysbiosis), followed by taking a (vaginal) sample from the recipient female after a predetermined amount of time, then determining which species and strains were successful in colonizing and engrafting in the recipient female's urogenital tract in vivo, e.g., by comparing the constituents (bacterial taxa and strains) of the donor female with those of the recipient female (e.g., by nucleic acid sequencing), and then upon obtaining the identification of strains that are successful in colonizing and engrafting the microbial niche in vivo, return to the healthy donor or a stored sample from the donor, from which the original material was prepared (or optionally the recipient's sample) to isolate those specific strains, propagate and formulate into bacterial preparations and/or (pharmaceutical) compositions. The provided methods thus remove the need for cumbersome and labor-intensive in vitro testing procedures and the uncertainty inherent in the current methods of having to predict from in vitro assays the ability to colonize an environment in vivo, thereby increasing the likelihood of identifying relevant, highly adapted, and successful strains capable of colonizing and engrafting in vivo. The forward approach of first administering a donor sample comprising a substantially complete vaginal microbiota preparation (also referred to as “SCVMP” herein), and subsequently identifying the specific species and strains that were capable of engrafting and improving the dysbiosis in the recipient's vaginal tract, will not only have a higher chance of identifying the therapeutically effective strains that are capable of improving dysbiosis, but also allow identification of strain consortia, e.g., preferred combinations of bacterial strains, which represent a sub-population of the donor sample, which provide for the therapeutic effect.

In some embodiments, the methods provided herein further comprise monitoring the recipient female for one or more biomarkers or symptoms of the underlying ailment (e.g., dysbiosis) and based upon an improvement of the one or more biomarkers or symptoms initiate the identification of strains (e.g., by comparing the resident strains of the recipient with those of the donor). In such embodiments, the species/strains that are identified are not only capable of colonizing and engrafting but are also associated with improvement of the underlying ailment. Thus, in some embodiments, strains successful in colonizing and engrafting are further selected by determining a health outcome in the female recipient.

The preparations of selected strain(s) (e.g., as described in 2) are administered locally, e.g., to the urogenital tract of the female human recipient, e.g., to the mucosal surfaces of the vaginal cavity,—not, as often described in the art, orally13 to increase successful colonization and engraftment.

While not explicitly excluded, the methods described herein generally do not rely on ex vivo culturing and/or propagating a recipient female's own microbiota for the purpose of re-introducing same after shifting the microbial community in vitro (e.g., from an abundance of dysbiotic to beneficial microbes). Rather, the methods described herein seek to exploit the competitive and other environmental pressures of the microbial niche in vivo (e.g., of the urogenital tract of a host of the microbial community, e.g., a recipient human female) to select and shift the administered donor microbial community to one that is dominated by lactobacilli typical for a healthy microbiome and that has successfully adapted to the in vivo selective pressures that exist in the recipient's niche. Thus, these methods generally do not rely on ex vivo approaches described above. Rather, donor strain(s) that are identified, isolated and/or propagated can be administered to various female recipients unrelated to the donor.

A healthy vaginal flora is characterized by an acidic environment inhabited predominantly by lactic acid bacteria, primarily species of Lactobacillus (residing in the vaginal microbial niche). The microbial composition in healthy women can differ, though it is typically dominated by one of four Lactobacillus species: L. crispatus. L. iners, L. gasseri, L. jensenii, and mixtures thereof. A healthy vagina of a women of child-bearing age is estimated to be dominated by 10⁷-10⁹ colony forming units of lactic acid producing bacteria (e.g., Lactobacillus) per gram of fluid. The species distribution differs between women of different geographical background, race (e.g., Asian, white women, black, Hispanic), age, lifestyle and the like. The composition of the vaginal flora is also influenced by which specific strains the woman has inherited from her mother and/or which strains have migrated from her digestive tract to the urogenital tract. Healthy, fertile women present with a pH of about 3 to 5.5 (more specifically between pH 3.5 and 4.5) in the vagina, primarily as a result of lactic acid production. Vaginal pH undergoes physiological changes from birth to menopause. The increase of vaginal pH above 4.0-4.5 is detrimental for the survival of Lactobacillus bacteria, but not for other microorganisms. The vaginal lactobacilli are believed to have a protective effect against vaginal colonization by pathogenic microorganisms (e.g., yeast (Candida albicans), Trichomonas vaginalis, Neisseria gonorrhoeae, and Chlamydia trachomatis, and viruses, e.g., HIV, HSV-2, and various anaerobes) and prevent the vaginal establishment of, for instance, bacteria that are present in the colon, such as Gardnerella vaginalis, Mobiluncus, Bacteroides, Prevotella and Escherichia coli.

Several factors may contribute to the disturbance of the vaginal flora. Factors may include, a) use of antibiotics to kill pathogenic bacteria which can lead to significantly reduced levels of lactobacilli in the vagina; b) hormonal changes, in particular changes in estrogen levels, which are observed in several phases of a woman's life (e.g., puberty, pregnancy, childbearing age, pre- and post-menopause); estrogen levels are thought to be associated with Lactobacillus levels (dominance) in the vagina; c) sexual intercourse, which can be associated with pH increases (semen generally is alkaline) that may disturb the vaginal flora, because bacteria other than lactobacilli may start to flourish once the vaginal pH increases; d) use of medications, e.g., chemotherapeutics or antimycotics; e) use of birth control products; f) during menstruation; g) insufficient hygiene (e.g., promoting undesirable spread of the microorganisms from rectum to the urogenital area); h) general health status, such as, e.g., being diabetic; and (i) use of excessive hygienic measures in particular the use of vaginal douches.

Disturbance of the vaginal flora may lead to vaginal dysbiosis and vaginal disorders, e.g., candidiasis and bacterial vaginosis, which are two common vaginal disorders that affect women worldwide. Bacterial vaginosis is believed to be the result of displaced vaginal lactic acid producing bacteria which are replaced by a range of unwanted species such as Gardnerella vaginalis, Bacteroides, Mobilumeus, Prevotella, and Mycoplasma hominis. Vaginal infections are most often associated with one or more of: Escherichia, Enterococcus, Pseudomonas, Proteus, Klebsiella, Streptococcus, Staphylococcus, Gardnerella, Ureaplasma, Bacteroides, Peptococcus, Neisseria, Serratia, Sneathia, Megasphera, Corynebacterium, Clostridium, and Candida.

Vaginal lactobacilli predominance is thought to play an important role in resistance to infection via production of lactic acid and acidification of the vagina and by production of other antimicrobial products, such as, e.g., hydrogen peroxide. The presence of lactobacilli in the vagina has been linked to decreased frequencies of bacterial vaginosis, yeast vaginitis and sexually transmitted pathogens, including Neisseria gonorrhea, Chlamydia trachomatis, and Trichomonas vaginalis. Lactobacillus dominance varies among ethnic groups (they are thought to be very predominant in Asian and white women but less so in black and Hispanic women, though they still represent the majority).

Studies have shown that Lactobacillus-depleted communities can be transient, lasting just a few days, while in other instances the depleted communities persist for many weeks Some women with Lactobacillus-depleted communities remain asymptomatic and healthy. However, such women may be at higher risk for infections and STDs.

Provided herein are methods of identifying one or more bacterial strains capable of colonizing the female genitourinary tract. The methods include: providing a donor sample comprising a SCVMP derived from a donor female comprising a plurality of bacterial strains; administering an effective amount of the donor sample to a recipient female's genitourinary tract (e.g., a female's genitourinary tract exhibits a dysbiotic vaginal microbial niche); assessing a desired change (e.g., a health change (e.g., of a symptom) and/or a change in a microbial community, e.g., the vaginal microbial niche) of the recipient female's genitourinary tract over a predetermined time period; and identifying one or more bacterial strains that colonize the vaginal microbial niche of the recipient female's genitourinary tract by analyzing a recipient sample comprising a plurality of bacterial strains obtained from the recipient female's genitourinary tract after achieving the desired change in dysbiosis.

The methods may further comprise (a) nucleic acid sequencing of the microbial constituents of the recipient sample after determining a desired change in dysbiosis, and of the donor sample, (b) comparing the sequencing results for the microbial constituents of the recipient sample with the microbial constituents of the donor sample to determine sequence identity of strains residing in both, and (c) determining that one or more strains is capable of colonization if the one or more strains identified from the recipient sample match one or more strains from the donor sample by a predetermined degree of sequence identity. The predetermined degree of sequence identity may be at least 95%, 96%, 97%, 98%, 99%, 99.7%, 99.8%, 99.9%, or 100%.

Aspects of the invention relate to methods of identifying one or more bacterial strains capable of colonizing and engrafting the female genitourinary tract. The methods include engrafting bacterial preparations, such as SCVMPs, derived from healthy human female donors in recipient human females (e.g., females who exhibit a dysbiosis), followed by taking a (vaginal) sample from the recipient female after a predetermined amount of time, then determining which species and strains were successful in colonizing and engrafting in the recipient female's urogenital tract in vivo, e.g., by comparing the constituents (bacterial taxa) of the donor female with those of the recipient female (e.g., by nucleic acid sequencing), and then upon obtaining the identification of strains that are successful in colonizing and engrafting the microbial niche in vivo, return to the healthy donor or a stored sample from the donor, from which the original material was prepared (or optionally the recipient's sample) to isolate those specific strains. In some embodiments, the methods provided herein further comprise monitoring the recipient female for one or more biomarkers or symptoms of the underlying ailment (e.g., dysbiosis) and based upon an improvement of the one or more biomarkers or symptoms initiate the identification of strains (e.g., by comparing the resident strains of the recipient with those of the donor). In such embodiments, the species/strains that are identified are not only capable of colonizing and engrafting but are also associated with improvement of the underlying ailment. Thus, in some embodiments, strains successful in colonizing and engrafting are further selected by determining a health outcome in the female recipient.

These methods include: (1) providing a donor sample comprising a SCVMP derived from a donor female comprising a plurality of bacterial strains; (2) administering an effective amount of the donor sample to a recipient female's genitourinary tract, e.g., that exhibits a dysbiotic vaginal microbial niche, and/or to a recipient female that exhibits one or more symptoms of a disease or disorder associated with the genitourinary tract (e.g., an infection, e.g., vulvovaginal candidiasis, bacterial vaginosis, or inflammation), e.g., itching, redness, discharge, malodor, etc.; (3) assessing a desired change in (a) the composition of the microbiota of the recipient female's genitourinary tract and/or (b) one or more disease-associated health symptoms (e.g., a reduction in dysbiosis and/or a reduction one or more disease-associated health symptoms) over a predetermined time period; and (4) identifying one or more bacterial strains that colonize the vaginal microbial niche of the recipient female's genitourinary tract by analyzing a recipient sample comprising a plurality of bacterial strains obtained from the recipient female's genitourinary tract after achieving the desired change in, e.g., dysbiosis and/or one or more disease-associated health symptoms.

In some embodiments, the methods further include (a) conducting nucleic acid sequencing of the microbial constituents of the recipient sample after determining the desired change, and of the donor sample, (b) comparing the sequencing results for the microbial constituents of the recipient sample with the microbial constituents of the donor sample to determine sequence identity of strains residing in both, and (c) determining that one or more strains is capable of colonization if the one or more strains identified from the recipient sample match one or more strains from the donor sample by a predetermined degree of sequence identity.

The steps may include, e.g., collecting a bacterial sample, such as vaginal fluid (vaginal secretion), e.g., from the vaginal tract of a female donor with a normal, healthy vaginal flora or from a recipient (e.g., a recipient exhibiting a dysbiosis and/or a disease or disorder associated with the urogenital tract). For nucleic acid sequencing, morphological analysis and/or in vitro propagation, a sample comprising microbiota can be taken, e.g., from the vaginal mucosa (e.g., from the donor female). For example, a sample is taken using a suitable sample collection tool, e.g., a menstrual cup (e.g., a Softcup) or by inserting a vaginal swab, into the vagina and, e.g., swabbing the vaginal lining. In some embodiments, the sample is a vaginal swab, a vaginal mucus sample, a vaginal tissue sample or a vaginal cell sample.

The sample is typically treated as specified for the detection method to be used. For example, to analyze nucleic acids from the collected sample, the swab is placed in PBS or a similar buffered liquid, and the cells are collected and lysed using standard methods (thus, the sample may comprise isolated DNA, RNA or total nucleic acids extracted from the microbial sample). In a preferred embodiment, the cervico-vaginal samples are diluted with saline to reduce the viscosity, aliquoted (for procedure, see Example 3) and stored at −80° C. prior to DNA extraction. In a preferred embodiment, human DNA is removed prior to sequencing. DNA may be extracted using the Molysis Complete5 kit (MolZym), which uses a differential lysis method to extract microbial DNA and remove human DNA.

For (morphological) observation of the bacterial cells (e.g., for assessment of vaginal health, e.g., to assess Amsel criteria or Nugent score), the sample can be applied to a glass slide and treated using standard methods (e.g., for staining and/or fixation). For bacterial isolation and propagation, the sample can be applied to an appropriate culture medium, e.g., a solid or liquid medium, and if desired, a medium selective for lactic acid producing bacteria, such as, e.g., Lactobacillus.

Samples should be obtained and maintained using appropriate procedures to maintain the composition of the bacterial strains as much as possible. Factors that should be monitored are, amongst others, temperature, humidity, and contact with air (oxygen). Suitable sampling methods are known in the art and can be identified by the person of skill without undue burden.

### SCVMP and Lactobacilli

Aspects of the invention relate to methods of identifying one or more bacterial strains capable of colonizing the female genitourinary tract comprising:
providing a donor sample comprising a SCVMP derived from a donor female comprising a plurality of bacterial strains;
administering an effective amount of the donor sample to a recipient female's genitourinary tract that exhibits a dysbiotic vaginal microbial niche;
assessing a desired change in dysbiosis of the recipient female's genitourinary tract over a predetermined time period;
identifying one or more bacterial strains of the donor sample that colonize the vaginal microbial niche of the recipient female's genitourinary tract by analyzing a recipient sample comprising a plurality of bacterial strains obtained from the recipient female's genitourinary tract after achieving the desired change in dysbiosis.

The donor sample comprising a SCVMPs described herein can be, e.g., administered to the vaginal cavity to modulate the vaginal microbial niche for maintenance of a healthy vaginal microbiota and to help restore an unbalanced vaginal microbiota and to identify the one or more bacterial strains that are capable of colonizing in the recipient's vaginal niche and effecting the desired change in dysbiosis in the recipient female.

The female urogenital (also known as genital-urinary) tract consists of interconnected biogeographical niches, such as the vaginal niche and the endometrial niche. Bacteria from the vaginal microbial community can migrate through the cervix to remote sites of the urogenital tract. A dysbiosis in the vaginal microbial community can result in a dysbiosis at remote sites. Dysbiosis at these sites has been associated with a range of disease and conditions, including urinary tract infection (UTI), pelvic inflammatory disease (PID), and bacterial vaginosis (BV). In some embodiments, administration of the donor sample comprising a SCVMP to the vagina includes resolving dysbiosis in remote sites of the urogenital tract.

In some embodiments, the donor sample comprising a SCVMP comprise one, two, three, four or five different bacterial species from the genus Lactobacillus. In some embodiments, the preparation comprises one, two, three, or four different bacterial species from the genus Lactobacillus. In some embodiments, the bacterial species comprise about 80%, about 85%, about 90%, about 91%, about 92%, about 93%, about 94%, about 95%, about 96%, about 97%, about 98%, about 99%, about 99.5%, about 99.9%, 80-99.9%, 75%-95%, 85%-95%, 85%-99.9%, or 90%-99.9% of the preparation (of the total of all detectable bacterial taxa (e.g., species) of the preparation).

In some embodiments, the donor sample comprising a SCVMP comprises of one of (a) to (o) Lactobacillus species and species combinations: a) Lactobacillus crispatus; (b) Lactobacillus iners; (c) Lactobacillus jensenii; (d) Lactobacillus gasseri; (e) Lactobacillus crispatus and Lactobacillus iners; (f) Lactobacillus crispatus and Lactobacillus jensenii; (g) Lactobacillus crispatus and Lactobacillus gasseri; (h) Lactobacillus iners and Lactobacillus jensenii, (i) Lactobacillus iners and Lactobacillus gasseri; (j) Lactobacillus jensenii and Lactobacillus gasseri; (k) Lactobacillus crispatus, Lactobacillus iners and Lactobacillus jensenii; (1) Lactobacillus crispatus, Lactobacillus iners and Lactobacillus gasseri; (m) Lactobacillus crispatus, Lactobacillus jensenii and Lactobacillus gasseri; (n) Lactobacillus iners, Lactobacillus jensenii and Lactobacillus gasseri: (o) Lactobacillus crispatus, Lactobacillus iners, Lactobacillus jensenii and Lactobacillus gasseri.

In one aspect, the donor sample comprising a SCVMP (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises less than 5% of Gardnerella spp., Atopobium spp., and Prevotella spp.; wherein the pharmaceutical composition comprises a pharmaceutically acceptable carrier or diluent.

In some embodiments, about 80-99.9% of all detectable bacterial species of the preparation comprise one bacterial species from the genus Lactobacillus, selected from Lactobacillus crispatus, Lactobacillus iners, Lactobacillus jensenii, and Lactobacillus gasseri. In some embodiments, about 80-99.9% of all detectable bacterial species of the preparation comprise Lactobacillus crispatus. In some embodiments, about 80-99.9% of all detectable bacterial species of the preparation comprise Lactobacillus iners. In some embodiments, about 80-99.9% of all detectable bacterial species of the preparation comprise Lactobacillus jensenii. In some embodiments, about 80-99.9% of all detectable bacterial species of the preparation comprise Lactobacillus gasseri.

In some embodiments, about 80-99.9% of all detectable bacterial species of the donor sample comprising a SCVMP comprise two bacterial species from the genus Lactobacillus, selected from Lactobacillus crispatus, Lactobacillus iners, Lactobacillus jensenii, and Lactobacillus gasseri. In some embodiments, about 80-99.9% of all detectable bacterial species of the preparation comprise Lactobacillus crispatus and Lactobacillus iners. In some embodiments, about 80-99.9% of all detectable bacterial species of the preparation comprise Lactobacillus crispatus and Lactobacillus jensenii. In some embodiments, about 80-99.9% of all detectable bacterial species of the preparation comprise Lactobacillus crispatus and Lactobacillus gasseri. In some embodiments, about 80-99.9% of all detectable bacterial species of the preparation comprise Lactobacillus iners and Lactobacillus jensenii. In some embodiments, about 80-99.9% of all detectable bacterial species of the preparation comprise Lactobacillus iners and Lactobacillus gasseri.

In some embodiments, about 80-99.9% of all detectable bacterial species of the preparation comprise three bacterial species from the genus Lactobacillus, selected from Lactobacillus crispatus, Lactobacillus iners, Lactobacillus jensenii, and Lactobacillus gasseri.

In some embodiments, about 80-99.9% of all detectable bacterial species of the preparation comprise Lactobacillus crispatus, Lactobacillus iners and jensenii. In some embodiments, about 80-99.9% of all detectable bacterial species of the preparation comprise Lactobacillus crispatus, Lactobacillus iners and gasseri.

In some embodiments, about 80-99.9% of all detectable bacterial species of the preparation comprise four bacterial species from the genus Lactobacillus, selected from Lactobacilhis crispatus, Lactobacillus iners, Lactobacillus jensenii, and Lactobacillus gasseri.

In some embodiments, Lactobacillus crispatus or Lactobacillus iners are present in greater relative quantities than other bacterial species of the preparation, for example, 80-99.9% of all detectable bacterial species of the donor sample comprising the SCVMP may be Lactobacillus crispatus, further comprising less than 20%, 10%, 5%, 2%, 1%, 0.5% or 0.1% other bacterial species.

### Further Lactobacillus Species

In some embodiments, one or more additional Lactobacillus species are present in the preparations of the donor sample in minor quantities (e.g., less than 20%, 15%, 10%, 5%, 2%, 1% of the Lactobacillus species of the preparation). In some embodiments, these lactobacilli species are present in a concentration of about 0.01-1%, 0.02-0.5% or 0.01-0.3% of all detectable bacterial species of the donor sample comprising the SCVMP.

In a further embodiment, the SCVMP comprises in the donor sample further lactobacilli other than Lactobacillus crispatus, Lactobacillus iners, Lactobacillus jensenii, or Lactobacillus gasseri, wherein the further Lactobacillus species include, e.g., Lactobacillus amylovorus, Lactobacillus gallinarum, Lactobacillus helveticus, Lactobacillusvaginalis, Lactobacillus acidophilus, Limosilactobacillus fermentum (formerly known as Lactobacillus fermentum), Lacticaseibacillus casei (formerly known as Lactobacillus casei), and Lacticaseibacilhes rhanmosus (formerly known as Lactobacillus rhamnosus). As used herein, Limosilactobacillus fermentum, Lacticaseibacillus casei, Lacticaseibacillus rhamnosus are referred to as Lactobacillus and are included in the meaning of Lactobacillus.

The SCVMP comprised in the donor sample comprises less than than 5% of Gardnerella spp., Atopobium spp., and Prevotella spp. In some embodiments, SCVMP comprised in the donor sample comprises less than 10%, less than 7%, less than 5%, less than 2%, less than 1%, less than 0.5%, or less than 0.1% of Gardnerella spp., Atopobium spp., and Prevotella spp. The preparation of the invention can further comprise less than 5% of Gardnerella vaginalis., Atopobium spp., and Prevotella spp. and Fannyhessa vaginae. Gardnerella vaginalis was historically misclassified and has recently been re-classified as belonging to the genus Bifidobacteria. The terms Gardnerella vaginalis and Bifidobacterium vaginalis are thus used interchangeably. Atopobium vaginae and Fannyhessea vaginea are also used interchangeably throughout the application.

As used herein, the SCVMPs are separate from the animal or human body (isolated SCVMPs or donor sample comprising said SCVMP). The SCVMP is thus used interchangeably with isolated SCVMP.

The donor sample comprising a SCVMP is suitable for administration, preferably vaginal administration, to a recipient subject. The recipient subject can be a human. In one embodiment, the SCVMP is suitable for vaginal administration. In a further embodiment, the SCVMP is for use in a method of treating dysbiosis, or bacterial vaginosis.

### Lactobacilli in the Vaginal Niche

Lactobacilli are the predominant microorganisms in the vaginal microbial community and they play a major role in maintaining a healthy urogenital tract, including the vaginal and/or endometrial niche. Lactobacilli are capable of preventing adhesion and growth of pathogenic microorganisms and/or overgrowth of pathobionts through mechanisms that appear to involve secretion of anti-adhesion factors, hydrogen peroxide, bacteriocins and fermenting the glycogen to lactic acid, thereby creating an acidic environment hostile to pathogens and pathobionts. The genus Lactobacillus comprises a phenotypically heterogenous group of Gram-positive, aerotolerant anaerobic, lactic acid producing bacteria. Other typical characteristics include being catalase-negative and rod-shaped, and generally possess DNA with a low content of guanine (G) and cytosine (C), less than about 50%. They are members of the phylum Firmicutes, class Bacilli, order Lactobacillus and family Lactobacilluseae. One skilled in the art will be able to identify Lactobacillus species using standard techniques.

Species of lactobacilli can be identified phenotypically, as well as genetically, e.g., on the basis of 16S rRNA (ribosomal RNA) sequence (or the DNA encoding the 16S rRNA, generally referred to as 16S rDNA). Genetic analysis can be performed using standard techniques, for example whole genome sequencing analysis as well as widely used typing approaches based on nucleotide variation in several hundred DNA sequences and a few gene fragments: Multi-locus Sequence Typing (MLST), Multi-locus Variable number of tandem repeats Analysis (MLVA), rMLST and cgMLST) discussed, e.g., in Marcos Pérez-Losada, M. et al., “Microbial sequence typing in the genomic era”, Infection, Genetics and Evolution, Vol. 63, September 2018, p. 346-359.

Other identification techniques include: Vaginal pH, Nugent Score, Whiff Test, gas liquid chromatographic analysis of glucose fermentation products, total anaerobe concentrations, total aerobe concentrations, enzymatic activity (e.g., lipase, phospholipase A2 and phospholipase C, hydrogen peroxide production).

In some embodiments, the SCVMPs comprised in the donor samples do not comprise (and are not derived from) isolated and/or culture-propagated bacterial strain(s). The preparations can be prepared from cervicovaginal secretions (vaginal fluid), e.g., collected from the vaginal tract of a female donor with a healthy vaginal flora. SCVMPs can be collected using standard techniques using commercially available collection devices, such as, a menstrual fluid collection device (soft cup or soft disc), a syringe, a tube, spatula or beaker, or an absorbent matrix. In some embodiments, the menstrual fluid collection device is a vaginal self-sampling device. A vaginal self-sampling device can be, e.g., used by donors to collect vaginal fluid or cervicovaginal secretions without the help of another person.

Optionally, the collected material is undergoing centrifugation. The centrifugation step may be performed to facilitate collection, without physical separation of vaginal fluid components. In some embodiments, the donor sample comprising a SCVMPs comprise one or more of: mucus (e.g., secreted by the cervix), shed epithelial cells, vaginal transudate, and bacteria other than lactobacilli found in the secretion from the female donor. It is thought that mucus and other components of the secretion (including other bacteria) are beneficial to Lactobacillus growth and survival upon administration to the urogenital tract thereby supporting engraftment in a female recipient. This provides an advantage over compositions comprising isolated strain(s). Thus, in some embodiments, the SCVMP is not cultured (e.g., for the purpose of strain isolation) or propagated in vitro but rather preferably stored in the refrigerator at 4° C. or immediately frozen after collection (e.g., frozen to 0° C., −20° C., −80° C., −190° C.), or optionally spray dried or lyophilized.

If desired, the cervicovaginal secretions can be further processed, e.g., prior to refrigeration or freezing, e.g., by filtration for sterility and/or to remove residual particles, aggregates and cells, and adding diluent, e.g, to arrive at a desired volume, concentration (e.g., CFU/mL) and/or viscosity, as discussed herein. Optionally, the SCVMP is kept refrigerated or frozen until it is formulated into a dosage form and/or dispensed into an applicator or dispenser.

The donor sample comprising a SCVMP comprises a substantially complete ecosystem of the donor's vaginal microbiota. Even lactobacilli that are present in very low quantities, such as 0.01% or even 0.001%, in certain embodiments, are successfully transferred to the recipient by administering the preparation. The administration of substantially all vaginal lactobacilli from the donor to the recipient allows the identification of the subset of species and strains that are capable of colonizing the recipient's vaginal niche and resulting in an improvement of the recipient's vaginal dysbiosis. Further, since even lactobacilli of very small relative quantities are transferred to the recipient female with the initial administration of the SCVMP, the methods of the invention will allow the identification of species and strains that colonize even if present at very low relative amounts. Further, the method of the invention allows the identification and preparation of a composition comprising a plurality of bacterial strains, e.g., a bacterial strain consortium, which is capable of engraftment and thus advantageous over the existing art, which uses single strains only based on in vitro data, which do not reliably predict the capacity of the single strains to engraft in the recipient's vaginal niche.

In some embodiments, the one or more strains present in the donor female or the donor sample (the sample obtained from the donor female), are identified to be present in the recipient female after administration of the SCVMP, wherein the one or more strains may be identified by sequencing methods, e.g., whole genome sequencing or haplotype analysis.

In some embodiments, at least a subset of the lactobacilli comprised in the SCVMPs described herein is capable of colonizing and becoming established (engrafted) in a human vagina. In some embodiments, colonization and engraftment occurs even during menstrual discharge. The lactobacilli comprised in the SCVMPs may continue to reside in the vagina after administration over several menstruation cycles. In some embodiments, the lactobacilli engraft in the vaginal microbial niche over one or more than one (e.g., two, three, four, five or six) menstruation cycle upon vaginal administration. Residence time can be determined, e.g., using nucleic acid sequencing, e.g., for specific lactobacilli, e.g., comparing sequencing results prior to and post administration of the SCVMPs described herein, e.g, to determine the identity of newly added members (e.g., one or more lactobacilli) to the recipient's microbial community, and then at predetermined time intervals (e.g., prior or post a menstruation cycle, optionally, over a number of menstruation cycles) determine (e.g., through nucleic acid sequencing) that all or a certain subset of the newly added members are still substantially present at the specific time interval. In some embodiments, the residence time of the recipient's microbiota may be compared to the donor's microbiota. The residence time may vary depending on various factors including hormone levels (e.g, estrogen), diet, sexual activity, acidity status of the vagina, and the presence or absence of genital infections and other microbial perturbations, e.g., treatment with antibiotics.

Successful colonization and engraftment of the lactobacilli comprised in the SCVMPs may be indicated by one or more of: decreased pH, increased lactic acid content, lower abundance of antibiotic resistance genes, decreased amount of fungal DNA, decreased toxin content, decreased pathogenicity factors, decreased inflammatory cytokines and chemokines, decreased immune cell infiltrates, decreased total bacterial DNA load, decreased total pathogenic DNA load, increased viscoelasticity, increased sialoglycan content, decreased relative or absolute abundance of pathobionts or pathogens, or any combination thereof in the vaginal cavity and the vaginal microbial niche (e.g., when compared to baseline of the same subject (e.g., prior to administration and engraftment) or a non-treated control subject).

### Stability

The lactobacilli comprised in the SCVMPs, one or more lactobacilli strains identified by the method of the invention, or compositions or pharmaceutical compositions comprising the same, are generally viable for up to one week if stored in the refrigerator at 4° C. and up to several months (e.g., 1, 2, 3, 4, 5, 6, 9, 12, 15, 18, or 24 months, or longer) if stored frozen at about −18° C., or preferably at about −80° C. Viability decreases over time. The SCVMPs, one or more lactobacilli strains identified by the method of the invention, or compositions or pharmaceutical compositions comprising the same are suitable for administration if they retain at least 30%, 40%, 50%, 60%, 70%, or at least 80% viable bacteria prior to use. Viable cells are generally able to colonize and engraft. Percent viability refers to the percentage of viable bacteria in a population. In some embodiments, the SCVMPs or one or more lactobacilli strains identified by the method of the invention, are further processed by adding a diluent, such as, e.g., saline, e.g., to formulate a pharmaceutical composition. In some embodiments, the SCVMPs, or one or more lactobacilli strains identified by the method of the invention, or compositions thereof described herein are further processed to be lyophilized (e.g., by freeze-drying, spray drying or other technologies known in the art), e.g., for easy storage, packaging, formulated in, for instance, compositions, and transport, and can be rehydrated before administration.

### Acidifying Agents

The compositions comprising a SCVMP described herein or one or more lactobacilli strains identified by the method of the invention, may optionally comprise one or more acidifying agents, such as, e.g., organic acids or salts thereof. In some embodiments, acidifying agents may be used to reduce the pH of the compositions, e.g., to below pH 5.5 or 5.0, e.g., to between pH 3.5 to 4.5, or pH 3.0 to 4.5. In decreasing pH such acidifiers may act as anti-microbial agents (e.g., to inhibit Candida or pathogenic bacteria). Acidifying agents comprise, e.g., lactic, acetic, ascorbic, citric, folic sorbic, or boric acid. In other embodiments, the acidifying agent is administered separate from the compositions comprising one or more lactobacilli strains identified by the method of the invention, or a SCVMP, e.g. prior to, concurrent with, or after administration of the composition (e.g., in a different dosage form, such as, e.g. a suppository, cream, gel, powder, douche or similar), e.g., for one to seven days, or one to ten days. In these embodiments, the acidifying agent may be used to promote the survival and engraftment of the one or more lactobacilli strains identified by the method of the invention, or the lactobacilli comprised in the SCVMP. Acidifying agents may also be useful as spermicides, e.g., to kill or inactivate residual sperm that may be present in the preparation. In one embodiment, the spermicidal activity is contributed by adding lactic acid. In one embodiment, lactic acid may be provided as a racemic mixture of D- and L-isomers, or at different suitable ratio, including, e.g., only D-lactate or only L-lactate.

In some embodiments, the compositions comprising one or more lactobacilli strains identified by the method of the invention, or a SCVMP is acidified by adding 0.1%, 0.5%, 1%, 1.5%, 2%, 2.5%, 3% or 5% of acidifier (e.g., lactic acid). In preferred embodiments, the acidifier is added at a concentration of 0.5% to 1.5%, or 0.2% to 2% (w/w), or a similar concentration that matches a healthy vagina.

### Viscosity

The donor sample or composition comprising a SCVMP described herein may comprise one or more pharmaceutically acceptable buffer or diluent or other excipients. In preferred embodiments, the diluent is saline (e.g., 0.9% NaCl), e.g., sterile normal saline. In one embodiment, the diluent is used to adjust the viscosity of the donor sample or composition, e.g., to lower the viscosity of the SCVMP derived from the cervicovaginal secretion, thereby increasing the ability to administer the composition to a female recipient, e.g., using an applicator or dispenser or similar vaginal delivery system. In other embodiments, one or more excipients is used to formulate the sample or composition in different dosage forms, such as, e.g., suppositories, creams or dissolving films or tablets.

In some embodiments, the dosage form is liquid, solid or semi-solid. The solid dosage form preferably comprises a tablet, capsule, or a film. The semi-solid dosage form preferably comprises a suppository, ointment, gel, cream or rigid foam. In a preferred embodiment, the dosage form is a gel.

When adjusting the viscosity for administration to a female recipient's vaginal cavity, e.g., using an applicator or dispenser, care should be taken to adjust the viscosity to be easily dispensable in the vaginal cavity (e.g., without having to apply significant force to expel the liquid composition), yet avoid making the donor sample or composition so fluid/liquid that is does not stay in the vaginal cavity for any appreciable amount of time after administration. Thus, a viscosity should be selected that is suitable to prevent rapid discharge from the vaginal cavity. The donor samples or compositions comprising a SCVMP described herein are preferably of a viscosity which allows the majority of it to stay in the vagina for at least 5 minutes, at least 10 minutes, at least 20 minutes, at least 30 minutes, when the female recipient is in an upright position, although administration is preferably carried out in a lithotomy position, e.g., with the female recipient lying down. Viscosity can be measured, e.g., using a Viscometer.

### Spermicides

The donor samples or compositions comprising a SCVMP described herein may comprise one or more spermicides. Similarly, compositions comprising one or more lactobacilli strains identified by the method of the invention may comprise one or more spermicides. Care should be taken when using nonoxynol-9 (N-9) because of its membrane disruptive properties (it increases the permeability of vaginal tissue by causing damage the cervicovaginal epithelium) and has been shown to be detrimental to Lactobacillus spp. Other spermicides include octoxynol-9, sodium cholate, and benzalkonium chloride. In one embodiment, lactic acid is used. In other embodiments, lactic acid may be used in combination, e.g., with citric acid.

### Prebiotics

A “prebiotic” as used herein is a growth substrate, which increases growth of bacteria (such as lactobacilli) comprised in the donor sample comprising a SCVMPs, or in a composition comprising one or more (isolated) bacterial strains identified according to the method of the invention, as could be measured, e.g., in vitro. If desired, though it is not generally necessary, a prebiotic may be added to the donor sample or compositions described herein, e.g., to create a synbiotic mixture, e.g., to increase growth of the bacteria of the preparation or the one or more bacterial strains upon administration to the genitourinary tract of a female recipient. This may, under certain circumstances, increase successful colonization and engraftment. In other embodiments, prebiotics may be used for the maintenance of an engrafted preparation and/or general vaginal health.

If a prebiotic is desired, it should be carefully chosen to not be greatly metabolizable by any yeast (e.g., Candida species), pathobionts or pathogens (e.g., E. coli and other Gram-negative bacteria) that may reside in the female recipient's genitourinary tract (e.g., to avoid promoting their growth). Prebiotics include, e.g., lactitol, lactulose, and in some instances also other oligosaccharides and soluble fibers, e.g., fructooligosaccharides (FOS), glucooligosaccharides (GOS), and inulin.

### Other Active Agents

If desired, other active agents may be added to the donor samples or compositions comprising one or more bacterial strains identified by the inventive method, e.g., to address bacterial or fungal infections and/or sexually transmitted diseases in the recipient female, for example antimicrobial agents, antifungal agents, antibacterial agents, antiviral agents, antibiotics, antiparasitic agents (e.g., with activities against Trichomonas vaginalis), anti-inflammatory agents, and the like. Care must be taken when formulating these agents into the pharmaceutical composition so as to not substantially interfere with the activity and efficacy of the lactobacilli comprised in the preparations and compositions described herein.

In some embodiments, the donor sample or compositions comprising one or more bacterial strains identified by the inventive method, comprise a form of estrogen. Adequate levels of estrogens play a role in the trophism of vaginal mucosa, and estrogens increase the cellular content of glycogen.

In some embodiments, the donor sample or compositions comprising one or more bacterial strains identified by the inventive method, comprise thiosulfate, e.g., to potentiate the anti-pathogenic effect of lactobacilli.

If desired, the donor sample or compositions comprising one or more bacterial strains identified by the inventive method, can further contain an antibiotic, such as, e.g., metronidazole, or one or more antibiotics of the following classes: a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a beta-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), and the like.

In some embodiments, the pharmaceutical compositions can further contain an antimicrobial (an antibiotic or antifungal) selected from metronidazole, tinidazole, secnidazole, clindamycin, nystatin, azithromycin, erythromycin, ofloxacin, doxycycline, levofloxacin, amoxicillin, and fluconazole.

If desired, the donor sample or compositions comprising one or more bacterial strains identified by the inventive method, can contain an agent for treating infections with mycobacteria. Suitable agents for treating infections with mycobacteria include an aminoglycoside (e.g., capreomycin, kanamycin, streptomycin), a fluoroquinolone (e.g. ciprofloxacin, levofloxacin, moxifloxacin), isozianid and isozianid analogs (e.g. ethionamide), aminosalicylate, cycloserine, diarylquinoline, ethambutol, pyrazinamide, protionamide, rifampin, and the like.

If desired, the donor sample or compositions can contain a suitable antiviral agent, such as remdesivir, oseltamivir, zanamavir, amantidine or rimantadine, ribavirin, gancyclovir, valgancyclovir, foscavir, Cytogam® (cytomegalovirus immune globulin), pleconaril, rupintrivir, palivizumab, motavizumab, cytarabine, docosanol, denotivir, cidofovir, and acyclovir.

If desired, the donor sample or composition can contain a suitable antifungal agent, such as polyene (e.g., nystatin and natamycin) and imidazole antifungals (e.g., flucanozole and clotrimazole).

If desired, the donor sample or compositions comprising one or more bacterial strains identified by the inventive method, can contain one or more suitable steroids. For example, the donor sample or composition may include androgens/anabolic steroids, estrogens, progestogens, corticosteroids, neurosteroids, estradiol, estropipate, premarin, drospirenone, noresthisterone, levonorgestrel, testosterone, fluoxymesterone, methylesterosterone, oxandrolone, and oxymetholone.

### Dosage Forms and Formulations

The SCVMP or the composition comprising one or more bacterial strains capable of colonizing and engrafting identified by the methods described herein, may be formulated into strain preparations and (pharmaceutical) compositions and various dosage forms. In the following, the preferred dosage forms are provided, wherein all dosage forms are equally suitable for SCVMPs and the one or more bacterial strains capable of colonizing and engrafting identified by the methods described herein.

Multiple dosage forms are contemplated herein, and include a suspension, spray, gel, cream, ointment, powder, (gelatin or vegetable cellulose) capsule, solution for lavages or douches, foams, films, ovules, a vaginal insert (e.g. tampon), tablets, disk, wafer (e.g., drying on film, by vaporization), or a microencapsulated product employing excipients and formulation techniques known to those skilled in the art. Particularly preferred dosage forms include formed gels, lyophilized gels, tablets, frozen formulations and films.

A number of suitable excipients can be used to formulate the SCVMP or the composition comprising one or more bacterial strains capable of colonizing and engrafting identified by the methods described herein, such as bulking agents, polymers, carbon sources, mucoadhesive agents, or pH modifiers and/or buffers.

The carbon source excipients may act as a carbon source for the microbiota contained in the SCVMP or the isolates bacterial strains identified by the method described herein. Such carbon courses comprise mannitol, maltodextran, and Guar gum. Some excipients may further serve as mucoadhesive agents or as viscosity agents. Bulking agents may comprise one or more of mannitol, micro-crystalline cellulose, maltodextran, guar gum, inulin, or alginic acid (e.g., sodium alginate). Polymers may comprise structural polymers. In some embodiments, polymers comprise one or more of mucin, hyaluronic acid, polyvinyl alcohol, sodium CMC, polyvinylpyrrolidone, hydroxypropyl methylcellulose, carbopol (e.g., Carbopol 934), and poloxamer (e.g., poloxamer 407). Mucoadhesive agents comprise, e.g., alginic acid (sodium alginate) and sodium CMC. Viscosity agents comprise, e.g., to Guar gum and Carbopol 934.

Some excipients serve as pH modifiers and/or buffers, such as lactic acid and acetate buffer.

Suitable formulations show little to no flow on suitable vertical surfaces and maintain high bacterial viability (e.g., CFU count) both upon formulation and during (long-term) storage.

Desired formulation selection parameters include, for example, mucoadhesion (of reconstituted product, e.g., in the vaginal tract); viscosity (of reconstituted product), e.g., final viscosity for gel-based product needs to be syringeable at ambient temperature and preferably congealed at 37° C. (at body temperature, e.g., in the vaginal tract); total sugar content (of reconstituted product), e.g., ideally at or lower than physiological concentration (about 0.5-1.0 mg/mL); volume of reconstituted product, e.g., up to 3 mL; hydration rate/disintegration rate (e.g., of gel/matrix), e.g., sufficient physical integrity to provide desired release rate; pH, e.g., between about pH 3.4-3.9 (e.g., to promote inhibition of competitive vaginal bacteria); water activity/moisture content (e.g., of dried formulations), e.g., between 0.5-3% water (e.g., for longer term dried formulation stability); microbial diversity, e.g., relative abundance of Lactobacillus species, such as, e.g., L. crispatus, L. gasseri, L. jensenii, and L. iners; total dose/potency, e.g., preferably at least about 1×10⁵ CFU/VCC, at least about 10⁶ CFU/VCC, at least about 10⁷ CFU/VCC or at least about 10⁸ CFU/VCC per administration (per dose), shelf-life (not reconstituted) at various temperatures, and microbial limits, e.g., absence of microorganisms such as, Pseudomonas aeruginosa, Candida albicans, Staphylococcus aureus, Ph Eur criteria 5.1.4, 2.6.12 & 2.6.13).

Suitable testing methods include standard assays, such as, plate count (e.g., MRS agar), e.g., for life bacteria count, dose determination, shelf-life; rheometer, e.g., for mucoadhesion and viscosity, pH meter, Karl Fisher/water activity meter, Ph Eur testing, e.g., for microbial loads.

The one or more bacterial strains identified by the methods of the invention, or the SCVMPs can be lyophilized and formulated into, e.g., gels and tablets as well as other dosage forms that can be filled with lyophilized products, such as, e.g., capsules. The composition comprising one or more bacterial strains identified by the methods of the invention or the SCVMPs can also be formulated into gels that can be frozen, as well as into liquid media (e.g., with glycerol) that can be frozen. The composition comprising one or more bacterial strains identified by the methods of the invention or the SCVMPs can also be formulated into (air-dried) films, that could be, e.g., shaped like disks. Losses in viability range from approximately 0.5 log to 1 log at the formulation step depending on excipient and dosage form.

In preferred embodiments, the composition comprising one or more bacterial strains identified by the methods of the invention or the SCVMP is provided in a dosage form selected from a tablet, pre-formed gel, lyophilized gel, liquid formulation, frozen formulation, film-forming formulation or film.

### Pre-Formed and Frozen Gels

The SCVMP or the composition comprising one or more bacterial strains capable of colonizing and engrafting identified by the methods described herein can be comprised in a pre-formed gel. In some embodiments, the pre-formed gel is provided in a vial, e.g., for drawing up into a syringe. In some embodiments, the pre-formed gel is provided in a suitable applicator. The pre-formed gel may be stored frozen or refrigerated to maintain the stability of the one or more bacterial strains identified by the methods of the invention or the SCVMP. In some embodiments, the pre-formed gel comprises hyaluronic acid. In some embodiments, hyaluronic acid is comprised in a concentration of about 0.3-3%. In some embodiments, hyaluronic acid is comprised in a concentration of about 0.5-2%. In some embodiments, the pre-formed gel comprising hyaluronic acid may be frozen at −80° C. In some embodiments, the frozen pre-formed gel comprising hyaluronic acid is substantially stable for a time period of at least 3 months, 6 months, 9 months, 12 months, 18 months or longer.

### Lyophilized Gels

The SCVMP or the composition comprising the one or more bacterial strains capable of colonizing and engrafting identified by the methods described herein can be comprised in in a lyophilized gel. The lyophilized gel is stable and may be stored for extended periods of time at 2-8° C. The lyophilized gel comprises the lyophilized SCVMP or the lyophilized composition comprising the one or more bacterial strains identified by the methods described herein and a gel forming excipient. The lyophilized gel may be supplied in a vial. The lyophilized gel can be reconstituted prior to administration (e.g., in a clinical or home setting) with a reconstitution agent. In some embodiments, the reconstitution agent comprises a gel, a gel forming agent, or a liquid. The liquid may comprise water, saline or another liquid suitable for reconstitution and subsequent administration to a subject.

In embodiments, the lyophilized gel comprising the one or more bacterial strains identified by the methods of the invention or the SCVMP further comprises sodium-carboxymethylcellulose (Na-CMC). In some embodiments, Na-CMC is comprised in a concentration of about 1-15%. In some embodiments, Na-CMC is comprised in a concentration of about 5-10%. In some embodiments, Na-CMC is comprised in a concentration of about 1-3%. In some embodiments, Na-CMC is comprised in a concentration of about 2%. In embodiments, the lyophilized gel comprises the one or more bacterial strains identified by the methods of the invention or the SCVMP and hyaluronic acid. In some embodiments, hyaluronic acid is comprised in a concentration of about 0.3-5%, 0.3-3%, or about 0.5-2%. In embodiments, the lyophilized gel comprises the one or more bacterial strains identified by the methods of the invention or the SCVMP and hyaluronic acid and sodium-carboxymethylcellulose (Na-CMC) at the above concentrations. The lyophilized gel comprising NA-CMP, hyaluronic acid, or a combination of both, is stable at 2-8° C. or at −20° C. for a time period of at least 3 months, 6 months, 9 months, 12 months, 18 months or longer. In some embodiments, the lyophilized gel comprising hyaluronic acid may be frozen at −80° C. In some embodiments, the frozen lyophilized gel comprising hyaluronic acid is stable for a time period of at least 3 months, 6 months, 9 months, 12 months, 18 months or longer. In some embodiments, the lyophilized gel and the reconstitution agent are provided as a kit.

### Film-Forming Formulations

The SCVMP or the composition comprising one or more bacterial strains capable of colonizing and engrafting identified by the methods described herein can be comprised in in a film-forming formulation. The one or more bacterial strains identified by the methods of the invention or the SCVMP can be formulated with polymeric excipients, wherein the polymeric excipients have bioadhesive properties and film-forming capacity. Exemplary polymeric excipients for film-forming formulations include polyvinyl alcohol (PVA), sodium lactate and lactic acid. In particular embodiments, the film-forming formulation comprises polyvinyl alcohol (PVA). In some embodiments, the PVA is comprised in a concentration of about 10-25%, 10-20%, or about 12-15%. In particular embodiments, the film-forming formulation comprises PVA in a concentration of about 12%. In some embodiment, the film-forming formulation comprises PVA, e.g. in a concentration of about 10-25%, 10-20%, 12-15% or 12%, and is air-dried. In some embodiments, the film-forming formulation is provided as a disc or wafer. In some embodiments, the film-forming formulation is provided as a mucoadhesive pessary or patch. The film-forming formulation is substantially stable for a time period of at least 3 months, 6 months, 9 months, 12 months, 18 months or longer at 2-8° C. In some embodiments, the film-forming formulation rapidly disperses or dissolves in contact with fluids, e.g. cervicovaginal solution, to intravaginally form a viscous and bioadhesive gel. In some embodiments, formation of a bioadhesive dispersion is retained in the vagina for prolonged periods of time.

### Tablets

The composition comprising one or more bacterial strains identified by the methods of the invention or the SCVMP can be comprised in in a tablet. The tablet may comprise agents with gel-forming properties, muco-adhesive properties, or a combination of both. The tablet may further comprise excipient, such as swelling agents, bulking agents, lactic acid, carbopol, HPMC, alginate, or sodium-carboxymethylcellulose (Na-CMC). In some embodiments, the bulking agent comprises microcrystalline cellulose, HPMC/PVP, maltodextran, or poloxamer 407. In particular embodiments, the tablet comprises the one or more bacterial strains identified by the methods of the invention or the SCVMP and Na-CMC. In particular embodiments, the tablet comprises the one or more bacterial strains identified by the methods of the invention or the SCVMP and polyvinylpyrrolidone (PVP). The most preferred excipients for tablets are sodium CMC, polyvinylpyrrolidone and poloxamer. The tablet may further be substantially stable, e.g., substantially retain Lactobacilli viability. The tablet may be substantially stable at 2-8° C. or at room temperature (about 25° C.) for a time period of at least 3 months, 6 months, 9 months, 12 months, 18 months or longer. In particular embodiments, the tablet comprises the one or more bacterial strains identified by the methods of the invention or the SCVMP and sodium-carboxymethylcellulose (Na-CMC) and is stable at 2-8° C. or at room temperature (about 25° C.) for a time period of at least 3 months, 6 months, 9 months, 12 months, 18 months or longer. In particular embodiments, the tablet comprises the one or more bacterial strains identified by the methods of the invention or the SCVMP and polyvinylpyrrolidone (PVP) and is stable at 2-8° C. or at room temperature (about 25° C.) for a time period of at least 3 months, 6 months, 9 months, 12 months, 18 months or longer.

### Liquid Formulations

The SCVMP or the composition comprising one or more bacterial strains capable of colonizing and engrafting identified by the methods described herein or the SCVMP can be comprised in in a liquid formulation. In some embodiments, the liquid formulation may be provided as a frozen formulation. In some embodiments, the liquid formulation is provided with gelling agents in a kit. The liquid formulation may comprise a cyroprotectant, such as glycerol, wherein the glycerol concentration is optionally less than 25%, less than 20%, less than 15%, or less than 10%. In some embodiments, the liquid formulation may be frozen, e.g., at −20° C. or −80° C., and substantially retains Lactobacillus viability. In some embodiments, the liquid formulation further comprises lactic acid. In a preferred embodiment, the liquid formulation comprises lactic acid and a cryoprotectant, such as glycerol. In some embodiments, the frozen liquid formulation comprising the cryoprotectant, and optionally lactic acid, is substantially stable for a time period of at least 3 months, 6 months, 9 months, 12 months, 18 months or longer. The most preferred excipients for frozen liquid gels are hyaluronic acid, poloxamer, and carbopol. In some embodiments, the liquid formulation comprising the composition comprising one or more bacterial strains identified by the methods of the invention or the SCVMP is provided with a gelling agent as a kit. In some embodiments, a frozen liquid formulation comprising the SCVMP is provided with a gelling agent as a kit. In some embodiments, a frozen liquid formulation which does not comprise the composition comprising one or more bacterial strains identified by the methods of the invention or the SCVMP is provided with a lyophilized gel as a kit, wherein the lyophilized gel comprises the one or more bacterial strains identified by the methods of the invention or the SCVMP.

### Lyophilized Formulations

The SCVMP or the composition comprising the one or more bacterial strains capable of colonizing and engrafting identified by the methods described herein, can be lyophilized. The lyophilized preparation or lyophilized composition is stable and may be stored for extended periods of time at 2-8° C. The lyophilized preparation or lyophilized composition comprises the lyophilized SCVMP or the lyophilized composition comprising the one or more bacterial strains identified by the methods described herein and a gel forming excipient. The lyophilized preparation or lyophilized composition may be supplied in a vial. The lyophilized preparation or lyophilized composition can be reconstituted prior to administration (e.g., in a clinical or home setting) with a reconstitution agent. In some embodiments, the reconstitution agent comprises a gel, a gel forming agent, or a liquid. The liquid may comprise water, saline or another liquid suitable for reconstitution and subsequent administration to a subject.

In particular embodiments, the lyophilized preparation or lyophilized composition comprising the one or more bacterial strains identified by the methods of the invention or the SCVMP further comprises Maltodextran. In some embodiments, Maltodextran is comprised in a concentration of about 1-10%, 2-8% or 5-7.5%. In some embodiments, Maltodextran is comprised in a concentration of about 4-7%, 4-6% or about 5%. In particular embodiments, the lyophilized preparation or lyophilized composition comprising the one or more bacterial strains identified by the methods of the invention or the SCVMP further comprises guar gum. In some embodiments, guar gum is comprised in a concentration of about 1-5%, 1-4% or 2-3%. In some embodiments, guar gum is comprised in a concentration of about 2.5-3.5% . . . . In particular embodiments, the lyophilized preparation or lyophilized composition comprising the one or more bacterial strains identified by the methods of the invention or the SCVMP further comprises polyvinyl alcohol (PVA). In some embodiments, PVA is comprised in a concentration of about 5-20%, 7-18% or about 9-15%. In some embodiments, PVA is comprised in a concentration of about 10%. In particular embodiments, the lyophilized preparation or lyophilized composition comprising the one or more bacterial strains identified by the methods of the invention or the SCVMP further comprises Polyvinylpyrrolidone. In some embodiments, Polyvinylpyrrolidone is comprised in a concentration of about 5-20%, or about 10-15%. In some embodiments, Polyvinylpyrrolidone is comprised in a concentration of about 10%. In particular embodiments, the lyophilized preparation or lyophilized composition comprising the one or more bacterial strains identified by the methods of the invention or the SCVMP further comprises hydroxypropyl methylcellulose. In some embodiments, hydroxypropyl methylcellulose is comprised in a concentration of about 10-20%, or about 12-15% In some embodiments, hydroxypropyl methylcellulose is comprised in a concentration of about 12%. In particular embodiments, the lyophilized preparation or lyophilized composition comprising the one or more bacterial strains identified by the methods of the invention or the SCVMP further comprises Poloxamer 407. In some embodiments, Poloxamer 407 is comprised in a concentration of about 10-40%, or about 15-30%. In some embodiments, Poloxamer 407 is comprised in a concentration of about 15-25% or about 20%. %. In particular embodiments, the lyophilized preparation or lyophilized composition comprising the one or more bacterial strains identified by the methods of the invention or the SCVMP further comprises Carbopol 934. In some embodiments, Carbopol 934 is comprised in a concentration of about 1-10%, or about 1-5%. In some embodiments, Carbopol 934 is comprised in a concentration of about 2-5%, 2-3%, or about 3%. In particular embodiments, the lyophilized preparation or lyophilized composition comprising the one or more bacterial strains identified by the methods of the invention or the SCVMP further comprises mannitol. In some embodiments, mannitol is comprised in a concentration of about 1-10%, or about 3-8%. In some embodiments, mannitol is comprised in a concentration of about 3-7.5%, or about 5%.

### Donor Considerations, Donor Sample Acquisition and Processing (Step 1)

Aspects of the invention include methods of identifying one or more bacterial strains capable of colonizing the female genitourinary tract by administering an effective amount of a donor sample comprising a SCVMP comprising a plurality of bacterial strains derived from a donor female to a recipient female's genitourinary tract that exhibits a dysbiotic vaginal microbial niche; assessing a desired change in dysbiosis of the recipient female's genitourinary tract over a predetermined time period; identifying one or more bacterial strains of the donor sample that colonize the vaginal microbial niche of the recipient female's genitourinary tract by analyzing a recipient sample comprising a plurality of bacterial strains obtained from the recipient female's genitourinary tract after achieving the desired change in dysbiosis.

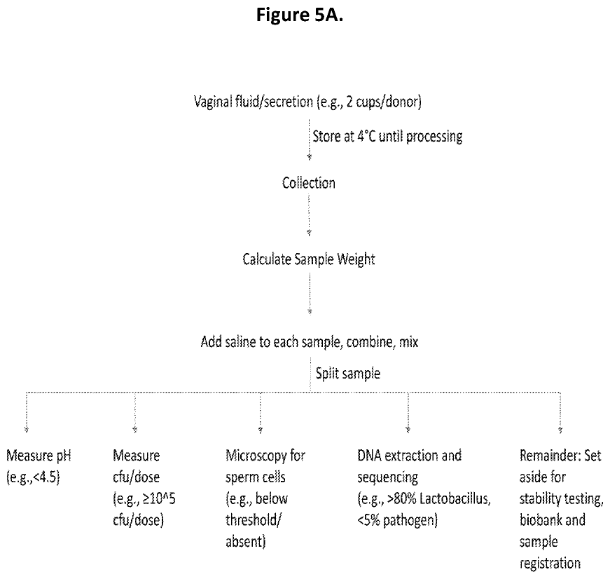

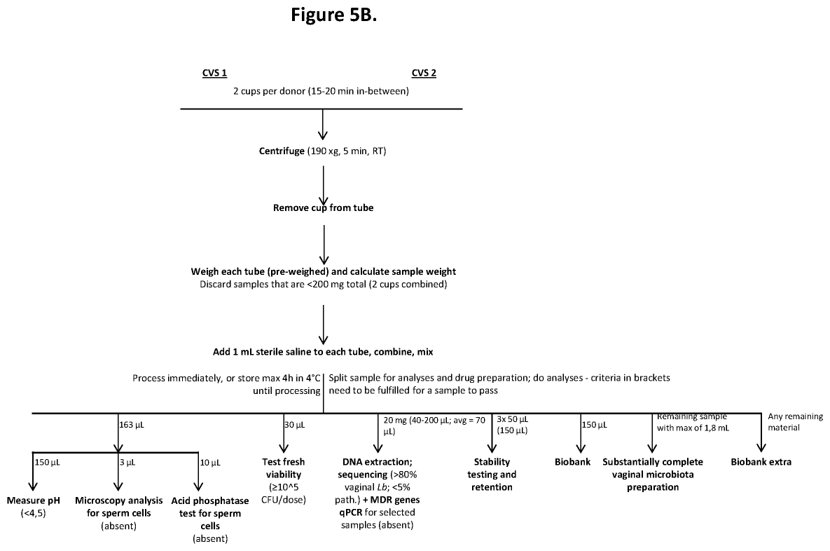

Donor samples (e.g., comprising a vaginal fluid/vaginal secretion) can be acquired and processed, e.g., as described in the examples (and summarized in FIGS. 5A and 5B) to provide SCVMPs Samples comprising a SCVMP can be prepared from cervicovaginal secretions (vaginal fluid), e.g., collected from the vaginal tract of a female donor with a healthy vaginal flora. Samples can be collected using standard techniques using commercially available collection devices, such as, a menstrual fluid collection device (soft cup), a syringe, a tube or beaker, or an absorbent matrix.

In some embodiments, the donor sample of a SCVMPs described herein do not comprise (and are not derived from) isolated and/or culture-propagated bacterial strain(s). SCVMPs can be prepared from cervicovaginal secretions (vaginal fluid), e.g., collected from the vaginal tract of a female donor with a healthy vaginal flora. SCVMPs can be collected using standard techniques using commercially available collection devices, such as, a menstrual fluid collection device (soft cup or soft disc), a syringe, a tube, spatula or beaker, or an absorbent matrix. In some embodiments, the menstrual fluid collection device is a vaginal self-sampling device. A vaginal self-sampling device can be, e.g., used by donors to collect vaginal fluid or cervicovaginal secretions without the help of another person.

Optionally, the collected material is undergoing centrifugation. The centrifugation step may be performed to facilitate collection, without physical separation of vaginal fluid components.

If desired, the cervicovaginal secretions can be further processed, e.g., prior to refrigeration or freezing, e.g, by filtration for sterility and/or to remove residual particles, aggregates and cells, and adding diluent, e.g., to arrive at a desired volume, concentration (e.g., CFU/mL) and/or viscosity, as discussed herein. Optionally, the SCVMP is kept refrigerated or frozen until it is formulated into a dosage form and/or dispensed into an applicator or dispenser.

In some embodiments, the engrafted lactobacilli persist for at least 1, 2, 3, 4, 5 or more menstrual cycles after administration of the SCVMP.

The SCVMP provided herein (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises less than 5% of Gardnerella spp., Atopobium spp., and Prevotella spp. For example, the methods of producing a SCVMP includes a step of providing a microbiota sample (such as a cervicovaginal secretion) from a healthy female donor and a step of releasing from quarantine (e.g., based on meeting one or more predetermined (quality) parameters, such as, e.g., those obtained from performing one or more of steps 5 (a) to 5 (e) below) a processed sample as a (pharmaceutical) composition, e.g., for administration to a female recipient in need of a SCVMP.

Generally, the step of providing and processing of a microbiota sample (such as a cervicovaginal secretion) from a healthy female donor can include one, two, three, four, five or more steps, and any combinations, e.g., any two, three, four, five or more steps, including: (1) adding a diluent (e.g., saline), buffer, or other excipient to the microbiota sample to create a diluted sample; (2) removing a portion of the diluted microbiota sample for testing (e.g., nucleic acid sequencing); (3) pre-cooling for either refrigeration or freezing of the remainder of the microbiota sample; (4) storing the refrigerated or frozen microbiota sample under quarantine, and/or (5) holding the refrigerated or frozen microbiota sample under quarantine until completion of any combination of (a), (b), (c), (d), and/or (e): (a) testing the donor to exclude the presence of transmissible pathogens (e.g., blood, vaginal swab, and/or urine sample testing), (b) confirming the composition and viability of the donor sample microbiota (e.g., lactobacilli), (c) further confirming the health of the female donor by a plurality of post-screening tests occurring within a time period of 30-90 days post-donation (or alternatively: 10-120 days, or 30-60 days), (d) testing for presence of sperm cells, and/or (e) testing pH of the sample. Any of steps (1) to (5) and 5 (a) to 5 (e) are optional and can be carried out sequentially or in parallel in any particular order that is desired.

In one embodiment, the method of producing the donor sample comprising a SCVMP comprises
A. providing a microbiota sample from a donor female genitourinary tract; wherein step A comprises one, two, or three of steps (1), (2), (3) or any combination thereof, and both steps (4) and (5):
   (1) adding a diluent to the microbiota sample to create a diluted sample,
   (2) removing a portion of the diluted microbiota sample for testing (e.g., nucleic acid sequencing),
   (3) pre-cooling for either refrigeration or freezing of the remainder of the microbiota sample,
   (4) storing the refrigerated or frozen microbiota sample under quarantine,
   (5) holding the refrigerated or frozen microbiota sample under quarantine until any completion of any combination of (a) testing the donor to exclude the substantial presence of one or more transmissible pathogens (e.g., blood, and/or cervicovaginal secretions, and/or urine sample testing), (b) confirming the composition and viability of the microbiota, or (c) further confirming the health of the female donor by a plurality of post-screening tests occurring within a time period of 30-90 days post-donation; and
B. releasing the refrigerated or frozen microbiota sample from quarantine to define the SCVMP.

Step (1) may include, e.g., adding an acidifying agent (e.g., to adjust the pH of the sample); adjusting the viscosity of the sample (e.g., to aid administration as described herein); adjusting the isotonicity/osmolarity; and/or adding one or more other active agents, such as described herein, including spermicides, antimicrobial agents, hormonal agents, anti-inflammatory agents, and optionally prebiotics. In one embodiment, the acidifying agent is lactic acid.

For step (2), the microbiota sample (e.g., a sample of vaginal fluid/vaginal secretion) is preferably at least 75 mg or 100 mg, more preferably at least 150 mg and a portion is removed for nucleic acid sequencing. The microbiota sample may be 75 mg, 100 mg, 150 mg, 200 mg, 250 mg, or 300 mg. In a further embodiment, the microbiota sample (e.g., a sample of vaginal fluid/vaginal secretion) is at least 200 mg, 300 mg, 400 mg, 500 mg, 500 mg, 700 mg or more. In one embodiment, the microbiota sample (e.g., a sample of vaginal fluid/vaginal secretion) is at least 500 mg.

Sequencing is performed to assess the microbial community of the donor microbiota sample and to select suitable donor females. Preferably, the presence of one, two, three, four or five different bacterial species from the genus Lactobacillus is detected in the donor microbiota sample by nucleic acid sequencing. Most preferably, one (dominant) bacterial species from the genus Lactobacillus is detected in the donor microbiota sample by nucleic acid sequencing. In some embodiments, nucleic acid sequencing determines that the donor microbiota sample comprises 80%, about 85%, about 90%, about 91%, about 92%, about 93%, about 94%, about 95%, about 96%, about 97%, about 98%, about 99%, about 99.5%, about 99.9%, 80-99.9%, 75%-95%, 85%-95%, 85%-99.9%, or 90%-99.9% lactobacilli of one species of the total of all detectable species in the preparation. In some embodiments, nucleic acid sequencing determines that the donor microbiota sample comprises 80%, about 85%, about 90%, about 91%, about 92%, about 93%, about 94%, about 95%, about 96%, about 97%, about 98%, about 99.9%, about 99.5%, about 99.9%, 80-99%, 75%-95%, 85%-95%, 85%-99.9%, or 90%-99.9% lactobacilli of more than one species (e.g., two, three, four, or five species) of the total of all detectable species in the preparation. Preferably, nucleic acid sequencing determines that the donor microbiota sample comprises species of Lactobacillus selected from: a) Lactobacillus crispatus; (b) Lactobacillus iners; (c) Lactobacillus jensenii; (d) Lactobacillus gasseri, or any combination thereof (including combinations of two, three, or all four species).

Optionally, nucleic acid sequencing is performed to identify any pathogens or pathobionts in the donor sample, e.g., to determine that a donor sample is substantially free of pathogens and pathobionts. For example, nucleic acid sequencing is preformed to detect the presence of one or more of Gardnerella spp., Atopobium spp., and/or Prevotella spp. In some embodiments, nucleic acid sequencing is preformed to detect the presence of one or more of Gardnerella spp., Atopobium spp., Prevotella spp., and/or Fannyhessa vaginae. In some embodiments, nucleic acid sequencing is performed to detect the presence of one or more of Gardnerella vaginalis, Bacteroides, Mobiluncus spp., Sneathia spp., and Mycoplasma hominis. In some embodiments, nucleic acid sequencing is preformed to detect the presence of one or more of Escherichia, Enterococcus, Pseudomonas, Proteus, Klebsiella, Streptococcus, Staphylococcus, Gardnerella, Ureaplasma, Bacteroides, Peptococcus, Neisseria, Serratia, Corynebacterium, Clostridium, and Candida. The presence of any one or more of these species suggests the displacement of healthy vaginal lactic acid producing bacteria by unwanted species, signaling the presence of vaginal dysbiosis. Donor samples containing more than about 1%, 3%, 5%, 8%, 10% or more than about 15% of species belonging to one or more unwanted species are not used for further processing to generate the SCVMPs described herein. In one embodiment, donor samples containing more than about 5% of species belonging to one or more unwanted species (e.g., are Gardnerella spp., Atopobinum spp., and/or Prevotella spp.) are not used for further processing to generate the SCVMPs described herein.

Optionally, nucleic acid sequencing is performed to identify the presence of any antimicrobial resistance (AMR) genes in the donor sample, e.g., to determine that a donor sample is substantially free of antimicrobial resistance (AMR) genes. Antimicrobial resistance (AMR) genes include genes that confer resistance to one or more antibiotics, including, e.g., aminoglycosides, beta-lactams, tetracyclines, and sulfonamides (e.g., as described and cataloged in the NCBI National Database of Antibiotic Resistant Organisms (NDARO)). It will be appreciated that due to extensive use of antibiotics the cut-off is not zero. Some reasonable allowance for the presence of AMR genes is made. The precise cut-off can be determined by one of ordinary skill, based on, e.g., the nature of the AMR genes (e.g., degree of health concern) and public health recommendations.

Alternatively, or in addition, tests are performed to determine that a donor sample is substantially free one or more of gram-negative toxins (e.g., endotoxin or lipopolysaccharide (LPS) and other secreted exotoxins and enterotoxins), pathogenicity factors/bacterial virulence factors, and/or colonization factors (e.g., motility, adherence, invasiveness, etc.).

Optionally, tests are performed (e.g., by microscopy) to identify the presence of any human sperm (spermatozoa) in the donor sample, e.g., to determine that a donor sample is substantially free of human sperm (spermatozoa).

Step (3) may include pre-cooling for either subsequent refrigeration (e.g., at 4° C.) or freezing (e.g., −18° C. or −80° C.), depending on the desired time period for storage, quarantine and use for administration. This step may also include freeze-drying (lyophilizing), e.g., for easy storage, packaging, formulation and transport. Further, this step may optionally include viability testing, e.g., upon refrigeration, freezing, or freeze-drying, e.g., viability of the lactobacilli comprised in the SCVMPs.

Steps (4) and (5) include storing the refrigerated, frozen or freeze-dried microbiota sample under quarantine, and holding the stored microbiota sample under quarantine until completion of a number of tests conducted on the donor microbiota sample and/or the sample donor. The quarantine is lifted, and the sample released for use as a SCVMP or composition (e.g., pharmaceutical composition), e.g., for administration to a recipient female, upon the sample and/or the donor passing one or more predetermined tests (sample quality and/or female donor health tests). Those include one or more of: (a) testing the donor to exclude the presence of transmissible pathogens (e.g., blood, vaginal swab, and/or urine sample testing); (b) confirming the composition and viability of the donor sample microbiota (e.g., lactobacilli), and/or (c) further confirming the health of the female donor by a plurality of post-screening tests occurring within a time period of 30-90 days post-donation (or alternatively: 10-120 days, or 30-60 days).

Step 5 testing to exclude the presence of transmissible (and potentially infectious) pathogens (e.g., blood, vaginal swab, and/or urine sample testing) may include determining that the female donor is substantially free of any one or more (two or more, three or more, or four or more) of: (i) bacteria involved in bacterial vaginosis (e.g., Gardnerella and Mobiluncus), (ii) yeast (e.g., Candida, Cryptococcus, and Saccharomyces species), (iii) sexually transmitted pathogens (including Neisseria gonorrhea, Chlamydia trachomatis, and Trichomonas vaginalis), (iv) bacteria involved in urinary tract infections (e.g., E. coli, Staphylococcus, Chlamydia, and Mycoplasma), and (v) viruses (e.g., HIV, human papilloma virus (HPV), hepatitis B virus, hepatitis C virus, HSV-2).

Step 5 testing may include determining that the female donor is substantially free of any one or more sexually transmitted infections or diseases (STI, STD), including Chlamydia, chancroid, crabs (pubic lice), genital herpes, genital warts, Hepatitis B, human immunodeficiency virus/acquired immunodeficiency syndrome, human papilloma virus, trichomoniasis, molluscum contagiosum, pelvic inflammatory disease, syphilis, gonorrhea, and yeast infections.

Step 5 testing may include determining that the female donor does not exhibit a dysbiosis in the vaginal tract, e.g., by one or more established tests for bacterial vaginosis (BV) and bacterial infections. Such tests include Amsel Criteria, Nugent Gram-stain scoring system, and Hay-Ison Criteria. Alternatively, other methods may be used to determine the absence of dysbiosis, e.g., using the BV Blue test or Affirm Microbial Identification Test.

Amsel Criteria include the presence of three of the following four symptoms: (a) thin homogeneous malodorous discharge; (b) vaginal pH fluid >4.5; (c) an amine odor from vaginal fluid when 10% KOH is added; and (d) the presence of “clue” cells (vaginal epithelial cells with adherent bacteria that obscure cell margins) (Amsel et al., Am. J. Med. 74:14-22 (1983)).

The Nugent Gram-stain scoring system involves assessment of a normally prepared Gram stain for relative abundance of three morphotypes of bacteria, and then calculating the so-called Nugent score based on the amounts of large Gram-positive rods (lactobacilli morphotype; decrease in lactobacilli is scored as 0 to 4), small Gram-negative and variable rods (Bacteroides and Gardnerella morphotype; scored as 0 to 4), and curved gram-variable rods (Mobiluncus spp. morphotype; scored as 0 to 2). The Nugent score can range from 0 to 10, with scores of 0-3 deemed normal (non-BV), 4-6 intermediate, and 7-10 positive for BV.

Hay-Ison Criteria (alternatively Ison-Hay scoring system) suggests five grades of flora: a) Grade 0, epithelial cells with no bacteria; b) Grade I, normal vaginal flora (Lactobacillus morphotypes alone); c) Grade II, reduced numbers of Lactobacillus morphotypes with a mixed bacterial flora; d) Grade III, mixed bacterial flora only, few or absent Lactobacillus morphotypes; c) Grade IV, Gram positive cocci only.

Grades 0, I, and IV are found in women without BV. Grade II is intermediate and not found in women with BV as defined by Amsel criteria. Grade III is consistent with BV as diagnosed by Amsel criteria. Grade Ill flora are indicative of BV (C. A. Ison and P. E. Hay, Sex Transm. Infect. 2002 December; 78 (6): 413-5).

The BV BLUE test (Gryphus diagnostics) detects sialidase activity, an enzyme produced by BV-associated bacteria such as Gardnerella vaginalis, Bacteroides spp., Prevotella spp., and Mobiluncus spp. A vaginal fluid sample is placed in the test vessel which contains a chromogenic substrate for sialidase. After incubation, a developer solution is added, and if the sample contained a high level of sialidase, a blue or green color is seen. Samples containing no sialidase, or low levels of this enzyme, will generate a yellow color in the reaction.

The AFFIRM Microbial Identification Test (Beckton Dickinson) is a DNA probe-based diagnostic test for the differential detection and identification of the three types of vaginitis-associated organisms: Candida spp., G. vaginalis and T. vaginalis.

In some embodiments, donor females are selected from generally healthy, pre-menopausal women, of ages 18 years and older with regular, predictable menstrual cycles. In some embodiments, donor females are selected from generally healthy, post-menopausal women. In some embodiments, donor females are selected from both pre- and post-menopausal women. Donors can take oral contraceptives, hormonal contraceptives, hormonal intrauterine devices or no contraceptives. Donors are substantially free of vaginal symptoms, such as odor, discharge, or itching. Optionally, donors do not use or perform one or more of (or all of a-e) during the sample donation period, e.g., from initial donor screening to the final donation: a) use vaginal feminine products that are inserted, e.g tampons, menstrual cups, sex toys, though sanitary napkins are acceptable; b) use other vaginal products, such as, e.g., cleansing products, spermicides, lubricants, hygiene powders and sprays); c) have vaginal and anal intercourse; d) take baths, go swimming, sit in a hot tub, and/or e) wear thong underwear.

Donors may be excluded if they exhibit one or more of the following (e.g., test above a set of predetermined thresholds, e.g., concerning viral, fungal, and bacterial pathogen and/or pathobiont load, for which individual maximal thresholds may be set, e.g., above zero, such as, e.g., being substantially free thereof): (a) a health history of one or more of: bacterial vaginosis, recurrent yeast infection, trichomoniasis, syphilis, human papilloma virus (HPV) including genital warts, high grade pap smear, herpes, pelvic inflammatory disease, recurrent urinary tract infection, and mycoplasma, or any combination thereof; (b) testing positive for one or more of: HIV, Hepatitis A/B/C, syphilis, Human T-lymphotrophic Virus (HTLV)-I/II, WNV, Epstein-Barr Virus (EBV), rubella, Toxoplasma gondii, Herpes Simplex Virus (HSV)-1/2, Chlamydia, gonorrhea, Mycoplasma genitalium, Trichomonas vaginalis, HPV, and other yeast or bacteria that are considered pathogenic/abnormal and/or show antibiotic resistance, or any combination thereof, (c) vaginal fluid/cervicovaginal secretions not dominated by one of the common vaginal Lactobacillus species, e.g., as determined by qPCR, and/or (d) history of gonorrhea or Chlamydia (e.g., within 12 months prior to screening), or any combination of (a), (b), (c) and (d).

Further, donors may be excluded if they exhibit one or more of the following: hysterectomy, intra-uterine device insertion or removal, cervical cryotherapy, or cervical laser treatment (e.g., within 2 months prior to screening), any condition requiring regular periodic use of systemic antibiotics, use of long-acting hormonal treatments, social, medical, or psychiatric condition, including history of drug or alcohol abuse, menopause (e.g., defined as more than 12 consecutive months of amenorrhea without another known cause), irregular menstrual cycles, use of other medication, or any combination thereof. Preferably, donors are not currently pregnant or breastfeeding.

If desired, donors exhibiting one or more of cytomegalovirus (CMV), Rubella, and Varicella Zoster Virus (VZV) IgG, or any combination thereof, will only be matched with CMV positive and/or Rubella and/or VZV positive recipients, or can be excluded.

### Methods of Administration

Aspects of the invention include methods for vaginal administration to a human female subject of a donor sample comprising a SCVMP described herein. The methods may include using a device for administration, e.g., these methods would normally be carried out by a healthcare provider (e.g., in a clinic). For example, the donor sample comprising a SCVMP is provided frozen, e.g., in a cryo-vial, then thawed and pre-heated to 37° C. and then dispensed into a syringe/applicator by a healthcare provider and then administered to a recipient. Alternatively, the methods include using an alternative dosage form described herein, such as, e.g., a suppository, tablet, capsule, film, cream, etc. The methods can, if desired, be carried out by the recipient herself, e.g., by self-administration (e.g., at home). The methods may also include other healthcare related activities, such as diagnosing a health issue and providing standard of care in addition to providing a SCVMP or composition described herein. The activities can include the one or more combination therapies provided herein. For example, the methods for administration described herein may further comprise administering antimicrobial agents, antifungal agents, antibacterial agents, antiviral agents, antibiotics, antiparasitic agents (e.g., with activities against Trichomonas vaginalis), anti-inflammatory agents, and the like.

In embodiments, in which administration is carried out using a device, the device typically includes an open end (e.g., a tip) for insertion into the vaginal cavity, and a dispensing end (e.g., a plunger or piston) to expel the composition through the open end. The administration steps include: a) introducing the open end into a vaginal cavity, b) expelling the composition into the vaginal cavity, c) removing the device from the vaginal cavity (after administering the desired dose). Administration is preferably carried out with the recipient being in a lithotomy position, e.g., with the female recipient in a lithotomy position. In some embodiments, the recipient is to remain in a lithotomy position for at least 5 minutes, at least 10 minutes, at least 20 minutes, at least 30 minutes before returning to an upright position to allow the SCVMP or composition sufficient residence time in the vaginal cavity, e.g., sufficient contact time with the mucosal or endometrial surfaces of the vagina. In some embodiments, the administration is carried out targeting areas high in the vaginal cavity, e.g., near the vaginal fornices.

If desired, the menstrual cycle of the recipient female is taken into account when determining the timing of administration. For example, the procedure may be avoided during menstrual discharge. In some embodiments, a time other than during menstrual discharge is preferred for carrying out the administration, including, e.g., during a time window that includes prior to ovulation and prior to menstrual discharge.

In some embodiments, the precise steps, timing, and length of the procedure varies between recipients (and is, e.g., determined by a healthcare provider) in order to provide optimal conditions for the bacteria (e.g., lactobacilli) comprised in the SCVMP to colonize and become established (engrafted) in the vagina of the recipient female.

### Administration to Recipient (Step 3)

Donor samples comprising the SCVMP, or a composition comprising one or more bacterial strains identified by the method of the invention, can be administered to a recipient female's urogenital tract by any method known in the art. Suitable methods use applicators or dispensers for administration. For example, an applicator or dispenser is inserted into the vaginal cavity and a vaginal microbial preparation (e.g., the processed vaginal secretion or the one or more strains identified as described herein) comprised in the applicator or dispenser is dispensed into the vaginal cavity, and the applicator or dispenser is then removed from the vaginal cavity (after administering the desired quantity, e.g., CFU). Administration is preferably carried out with the recipient lying down, e.g., being in a lithotomy position. In some embodiments, the recipient is to remain in a lithotomy position for at least 5 minutes, at least 10 minutes, at least 20 minutes, at least 30 minutes before returning to an upright position to allow the SCVMP or composition sufficient residence time in the vaginal cavity, e.g., sufficient contact time with the mucosal or endometrial surfaces of the vagina. In some embodiments, the administration is carried out targeting areas high in the vaginal cavity, e.g., near the vaginal fornices. In some embodiments, the administration is carried out targeting areas high in the vaginal cavity, e.g., near the vaginal fornices. The methods may include using a device for administration, e.g., these methods would normally be carried out by a healthcare provider (e.g., in a clinic). For example, the composition comprising a SCVMP is provided frozen, e.g., in a cryo-vial, then thawed and pre-heated to 37° C. and then dispensed into a syringe/applicator by a healthcare provider and then administered to a recipient. Alternatively, the methods include using an alternative dosage form described herein, such as, e.g., a suppository, tablet, capsule, film, cream, etc. The methods can, if desired, be carried out by the recipient herself, e.g., by self-administration (e.g., at home) The methods may also include other healthcare related activities, such as diagnosing a health issue and providing standard of care in addition to providing a SCVMP or composition described herein. The activities can include the one or more combination therapies provided herein. For example, the methods for administration described herein may further comprise administering antimicrobial agents, antifungal agents, antibacterial agents, antiviral agents, antibiotics, antiparasitic agents (e.g., with activities against Trichomonas vaginalis), anti-inflammatory agents, and the like. In embodiments, in which administration is carried out using a device, the device typically includes an open end (e.g., a tip) for insertion into the vaginal cavity, and a dispensing end (e.g., a plunger or piston) to expel the composition through the open end. The administration steps include: a) introducing the open end into a vaginal cavity, b) expelling the composition into the vaginal cavity, c) removing the device from the vaginal cavity (after administering the desired dose).

If desired, the menstrual cycle of the recipient female is taken into account when determining the timing of administration. For example, the procedure may be avoided during menstrual discharge. In some embodiments, a time other than during menstrual discharge is preferred for carrying out the administration, including, e.g., during a time window that includes prior to ovulation and prior to menstrual discharge.

In some embodiments, the precise steps, timing, and length of the procedure varies between recipients in order to provide optimal conditions for the vaginal bacterial preparation (e.g., the processed vaginal secretion or the one or more strains identified as described herein) to colonize and become established (engrafted) in the vagina of the recipient female.

### Recipient's Health Symptoms and Biomarker Assessment (Step 4)

In some embodiments, successful colonization and engraftment is associated with changes in (a) the composition of the microbiota of the recipient female's genitourinary tract (e.g. the vaginal and or endometrial tract) and/or (b) one or more disease-associated health symptoms over a predetermined time period. These desired changes may be included as additional selection criteria for the one or more strains that are identified as being capable of colonization and engraftment. In a preferred embodiment, the one or more strains identified by the methods described herein are not only capable of colonizing and engrafting in the urogenital tract but also are associated with desired changes in the composition of the microbiota of the recipient female's genitourinary tract and/or (b) one or more disease-associated health symptoms. Such strains may preferably be selected for isolation, propagation and formulation into preparations and (pharmaceutical) compositions.

Desirable changes of the composition of the microbiota of the recipient female's genitourinary tract that can be determined include, e.g., a reduction in the relative abundance of pathogen and/or pathobiont residing in the genitourinary tract (e.g., less than 10% relative abundance of pathogen and/or pathobiont), and/or an increase in relative abundance of Lactobacillus above 50% (above 60%, or above 70%).

Desirable changes in one or more disease-associated health symptoms that can be determined include, e.g., (a) a reduction in one or more pro-inflammatory markers (e.g., local or systemic cytokines and chemokines) and/or decreased immune cell infiltrates; (b) a lowering of vaginal pH (e.g., by at least pH 0.3, 0.5, 1.0, or 1.5) and/or increased lactic acid content of the vaginal tract; and/or (c) a change in grade based on the Ison-Hay scoring system (e.g., to grade 0 or grade I), when compared to the values of (a), (b), and/or (c) determined in the same subject prior to administration of one or more strains capable of colonization and engraftment.

Other desirable changes in one or more disease-associated health symptoms that can be determined include, lower abundance of antibiotic resistance genes, decreased amount of fungal DNA, decreased toxin content, and/or decreased pathogenicity and/or virulence factors.

The vaginal health status of a female recipient (and also of a donor) can be assessed prior to and/or after administration at predetermined intervals. Testing may include using one or more of: Amsel Criteria, Nugent Gram-stain scoring system, and Hay-Ison Criteria.

Amsel Criteria include the presence of three of the following four symptoms: (a) thin homogeneous malodorous discharge; (b) vaginal pH fluid >4.5; (c) an amine odor from vaginal fluid when 10% KOH is added; and (d) the presence of “clue” cells (vaginal epithelial cells with adherent bacteria that obscure cell margins) (Amsel et al., Am. J. Med. 74:14-22 (1983)).

The Nugent Gram-stain scoring system involves assessment of a normally prepared Gram stain for relative abundance of three morphotypes of bacteria, and then calculating the so-called Nugent score based on the amounts of large Gram-positive rods (lactobacilli morphotype; decrease in lactobacilli is scored as 0 to 4), small Gram-negative and variable rods (Bacteroides and Gardnerella morphotype; scored as 0 to 4), and curved gram-variable rods (Mobiluncus spp. morphotype; scored as 0 to 2). The Nugent score can range from 0 to 10, with scores of 0-3 deemed normal (non-BV), 4-6 intermediate, and 7-10 positive for BV.

Hay-Ison Criteria (alternatively Ison-Hay scoring system) suggests five grades of flora: a) Grade 0, epithelial cells with no bacteria; b) Grade I, normal vaginal flora (Lactobacillus morphotypes alone); c) Grade II, reduced numbers of Lactobacillus morphotypes with a mixed bacterial flora; d) Grade III, mixed bacterial flora only, few or absent Lactobacillus morphotypes; e) Grade IV, Gram positive cocci only.

Grades 0, I, and IV are found in women without BV. Grade II is intermediate and not found in women with BV as defined by Amsel criteria. Grade III is consistent with BV as diagnosed by Amsel criteria. Grade III flora are indicative of BV (C. A. Ison and P. E. Hay, Sex Transm. Infect. 2002 December; 78 (6): 413-5).

Further desirable changes in one or more disease-associated health symptoms that can be determined include beneficial immune response, which may be assessed by e.g. proteomics (e.g., Olink) and transcriptomics (e.g., Fluidigm or RNAseq).

In some embodiments, the desired changes associated with successful colonization and engraftment in the recipient female's genitourinary tract are assessed within one, two, three, four, six, eight, ten, or twelve week(s), or 6 months, 8 months, 10 months, 12 months, 18 months, or within 24 months post administration of the donor sample.

In some embodiments, the desired changes associated with successful colonization and engraftment in the recipient female's genitourinary tract are assessed within 1, 2, 3, 4, 5, 6 or 7 day(s) post administration of the donor sample.

In some embodiments, the desired changes associated with successful colonization and engraftment in the recipient female's genitourinary tract are detectable within 1, 2, 3, 4, 5, 6, or 7 day(s) post administration of the donor sample.

In some embodiments, the desired changes associated with successful colonization and engraftment in the recipient female's genitourinary tract are detectable within one, two, three, four, six, eight, ten, or twelve week(s), or 6 months, 8 months, 10 months, 12 months, 18 months, or within 24 months post administration of the donor sample.

### Strain Identification (Step 5)

The advantageous effect of the administration of the donor sample of a SCVMP might be due to a subset of the bacterial strains present in the sample. Not much is known about the bacterial strains present in the vaginal niche and their interaction with each other. Without being bound by theory, it is believed that the beneficial microbial phenotype is strain-specific, e.g. while a first strain of a Lactobacillus species provides an advantageous effect on the dysbiotic vaginal microbiota of the recipient subject, a second strain of the same Lactobacillus species may not exert the same therapeutic effect to the same degree or might even result in adverse outcomes. While the precise characterization of what constitutes a “strain” is ill-defined in the field, the concept of “strain” is used in the present context to be a sub-species categorization based on genomic markers and/or sequences, for example, Yan Y et al. Strain-level epidemiology of microbial communities and the human microbiome, Genome Medicine vol 12, Art. No. 71 (2020). In one embodiment, strains are represented by the bacterial haplotypes. In another embodiment, strains are analyzed and defined by whole genome sequencing.

The methods of identifying one or more bacterial strains capable of colonizing and engrafting the female genitourinary tract include identifying one or more bacterial strains that colonize the vaginal microbial niche of the recipient female's genitourinary tract by analyzing a recipient sample comprising a plurality of bacterial strains obtained from the recipient female's genitourinary tract after administration of the donor sample comprising the SCVMP. As described herein, a sample for analysis can be taken from the recipient after achieving a desired change in the female recipient, e.g., a reduction in dysbiosis and/or one or more disease-associated health symptoms. As described, the methods further include (a) conducting nucleic acid sequencing of the microbial constituents of the recipient sample after determining the desired change, and of the donor sample, (b) comparing the sequencing results for the microbial constituents of the recipient sample with the microbial constituents of the donor sample to determine sequence identity of strains residing in both, and (c) determining that one or more strains are capable of colonization if the one or more strains identified from the recipient sample match one or more strains from the donor sample by a predetermined degree of sequence identity. In some embodiments, the predetermined degree of sequence identity of a strain is at least 98%, 98.5%, 99%, 99.5%, 99.8%, or 99.9%.

### Nucleic Acid Sequencing

In one embodiment, methods include sequencing of nucleic acids in the sample to identify the bacterial taxa present in the sample, e.g. a sample from the recipient female's genitourinary tract prior to and after administration of the donor sample, or a composition comprising one or more isolated bacterial strains (i.e. an isolate). Genetic information from the sample can be obtained by nucleic acid extraction from the sample. Methods for extracting nucleic acid from a sample are known in the art. For sequencing, nucleic acid is extracted from the bacterial samples collected, e.g., a vaginal fluid sample from a healthy female donor. Numerous standard DNA extraction protocols exist. Preferably, extraction protocols are used that specifically enrich for microbial DNA (e.g., protocols that reduce the amount of human DNA in the sample). Exemplary commercial kits include MoLysis Complete5 kit, and Qiagen DNAeasy Blood & Tissue kit. I In a preferred embodiment, human DNA is removed prior to sequencing. DNA may be extracted using the Molysis Complete5 kit (MolZym), which uses a differential lysis method to extract microbial DNA and remove human DNA.

Nucleic acid sequencing may be conducted by any method known in the art, including whole genome sequencing or other standard nucleic acid techniques, such as Southern blot, sequence analysis, electrophoresis, and PCR (including quantitative PCR (qPCR)). Sequences can be determined, e.g., by Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry (MALDI-TOF/MS), and sequencing by hybridization. Examples of electrophoretic analysis include slab gel electrophoresis such as agarose or polyacrylamide gel electrophoresis, capillary electrophoresis, and denaturing gradient gel electrophoresis.

Other exemplary sequencing methods include: dideoxy sequencing reactions (Sanger method) using labeled terminators or primers and gel separation in slab or capillary, shotgun sequencing, polymerase chain reaction (PCR), real-time polymerase chain reaction (qPCR), reverse transcription PCR (RT-PCR), multiplex PCR, ligase chain reaction, pyrosequencing, sequencing by synthesis, sequencing by ligation, massively parallel signature sequencing, polony sequencing, SOLID sequencing, DNA nanoball sequencing, mass spectrometry sequencing, microfluidic sequencing, high-throughput sequencing, Illumina sequencing, HiSeq sequencing, MiSeq sequencing, 16S ribosome sequencing, sequencing by chain termination and gel separation, as described by Sanger et al., PNAS, 74 (12): 5463 67 (1977); chemical degradation of nucleic acid fragments. See, Maxam et al., PNAS, 74:560 564 (1977); sequencing by hybridization. See, e.g., Harris et al., (U.S. patent application number 2009/0156412); Illumina (MiSeq and HiSeq), Helicos True Single Molecule Sequencing (tSMS) (Helicos Biosciences) See Harris T. D. et al. (2008) Science 320:106-109; see also, e.g., Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS, 100:3960-3964 (2003); 454 sequencing, e.g., Roche 454 GS FLX (Roche) (Margulies, M et al. 2005, Nature, 437, 376-380); SOLID technology, e.g., SOLID 5500 series (Applied Biosystems); Ion Torrent/IonProton/Ion Proton sequencing, e.g., PGM, Proton, S5 Series, single molecule, real-time (SMRT) technology (Pacific Biosciences); nanopore sequencing (Oxford Nanopore Technologies) (Soni G V and Meller A. (2007) Clin Chem 53:1996-2001); Qiagen Gene Reader; chemical-sensitive field effect transistor (chemFET) arrays (See e.g., US Patent Application Publication No. 2009/0026082); and use of an electron microscope (Moudrianakis E. N. and Beer M. PNAS USA. 1965 March; 53:564-71), or combinations thereof, incorporated by reference herein. Exemplary next generation sequencing methods are described, e.g., in Muneer Ahmad Malla et al., “Exploring the Human Microbiome: The Potential Future Role of Next-Generation Sequencing in Disease Diagnosis and Treatment”, Front. Immunol., 7 Jan. 2019, incorporated by reference herein). If desired, the extracted nucleic acids can be amplified. Suitable amplification methods include polymerase chain reaction (PCR), ligase chain reaction (LCR), loop-mediated isothermal amplification (LAMP), strand displacement amplification (SDA), replicase-mediated amplification, Immuno-amplification, nucleic acid sequence-based amplification (NASBA), self-sustained sequence replication (3SR), rolling circle amplification, and transcription-mediated amplification (TMA). For example, the PCR can be real-time PCR. In some embodiments, the PCR is quantitative real-time PCR (QRT-PCR).

If desired, nucleic acids synthesized as the result of gene transcription and/or metagenomic molecules can be detected. For example, in the case of the 16S rRNA gene, genomic DNA corresponding, in whole or part, to regions of the 16S rRNA gene, messenger RNA (mRNA) transcripts, in whole or part, of the 16S rRNA gene, and/or functional 16S IRNA may be detected and used to enumerate the abundance of a microbial taxon, e.g., characterized by sequence homology of a particular 16S rRNA gene sequence.

Genetic analysis can be performed using standard techniques, for example whole genome sequencing analysis as well as widely used typing approaches based on nucleotide variation in several hundred DNA sequences and a few gene fragments: Multi-locus Sequence Typing (MLST), Multi-locus Variable number of tandem repeats Analysis (MLVA), rMLST and cgMLST) (discussed, e.g., in Marcos Pérez-Losada, M. et al., “Microbial sequence typing in the genomic era”, Infection, Genetics and Evolution, Vol. 63, September 2018, p. 346-359).

Sequencing by any of the methods described above and known in the art produces sequence reads. Sequence reads can be analyzed according to any number of methods known in the art to identify microbes (including bacterial taxa) in a sample, e.g., a sample of vaginal fluid from a healthy donor.

Identification of microorganisms and sorting of them into taxa may also be achieved by other means such as analyzing proteomes, transcriptomes, metabolomes, or combinations thereof. For example, microbial RNA transcripts, proteins, non-16S genes, etc. may be profiled.

Other methods that may be employed to identify microbes include microarrays or other oligonucleotide probe-based technology (DNA or RNA-based) or antibody-based detection methods, including enzyme-linked immunoabsorbent assay (ELISA), western blot, immunohistochemistry, immunocytochemistry, flow cytometry and fluorescence-activated cell sorting (FACS), immunoprecipitation, and enzyme linked immunospot (ELISPOT).

### Strain Identity and Donor-Recipient Comparison of Resident Strains

Comparing the sequencing results for the microbial constituents of the recipient sample with the microbial constituents of the donor sample to determine sequence identity of strains residing in both can be done by any suitable method known in the art.

Typically, strain identity is established by comparing two nucleic acid sequences, e.g, one nucleic acid sequence obtained from the recipient sample and one obtained from the donor sample. One sequence acts as a reference sequence (e.g., from the donor sample), to which test sequences are compared (e.g., from the recipient sample). When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window can be established, and the sequences can be aligned. Methods of alignment of sequences for comparison are well-known in the art Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA) or by manual alignment and visual inspection (see, e.g., Current Protocols in Molecular Biology (Ausubel et al., eds. 1995 supplement)).

Algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available on the World Wide Web through the National Center for Biotechnology Information. The BLAST algorithm also performs a statistical analysis of the similarity between two sequences (see, e.g., Karlin & Altschul, Proc. Nat'l. Acad. Sci. USA 90:5873-5787 (1993)).

Another example of a useful algorithm is PILEUP which creates a multiple sequence alignment from a group of related sequences using progressive, pairwise alignments to show relationship and percent sequence identity PILEUP uses a simplification of the progressive alignment method of Feng & Doolittle, J. Mol. Evol. 35:351-360 (1987).

Several alternative approaches may be taken, if desired, to determine strain similarity and/or identity between donor and recipient. One approach is to isolate strain(s) from both a donor and a recipient and compare their sequences. Based on the degree of sequence similarity (very high) of the strain(s) a determination can be made that the strain(s) are clonal isolates. A second approach is to use metagenome assembled sequences or MAGs (metagenome-assembled genomes), a process that relies on unique sequences and co-abundance variation. This process does not require first to isolate strains to get their genome. The process supports the construction of a single strain genome from the metagenome sequencing data. The process requires a high number of bacterial sequencing reads (e.g., using a high-quality DNA purification preparation). Optionally, these approaches can be combined with one or more bioinformatics methods that filter out human reads. Exemplary approaches and techniques are described, e.g., in Johanna B. Holm et al., “Comparative Metagenome-Assembled Genome Analysis of “Candidatus Lachnocurva vaginae”, Formerly Known as Bacterial Vaginosis-Associated Bacterium-1 (BVAB1)”, Front. Cell. Infect. Microbiol., 31 Mar. 2020; Min Yap et al. “Evaluation of methods for the reduction of contaminating host reads when performing shotgun metagenomic sequencing of the milk microbiome”, Scientific Reports volume 10, Article number: 21665 (2020); H Bjørn Nielsen et al., “Identification and assembly of genomes and genetic elements in complex metagenomic samples without using reference genomes”. Nat Biotechnol. 2014 August; 32 (8):822-8.

### Repetition of Steps, Multiple Donors

It will be appreciated that the various method steps described here for identifying one or more bacterial strains capable of colonizing the human female genitourinary tract, can be, where appropriate and unless specified otherwise, performed in any particular order, and repeated, e.g., to improve selection of strains. For example, the steps of isolating vaginal fluid/vaginal secretions from donor and/or recipients, administrating processed vaginal fluid/vaginal secretions and/or identified strains to recipients, identifying strains (e.g., by sequencing of samples and/or by isolating and sequencing strains from samples) and performing one or more in vitro assessments, and other activities may be repeated one or more (e.g., two, three, or four) times. For example, a vaginal fluid/vaginal secretion is processed from a donor and administered to a recipient. After a period of time, the recipient is assessed, e.g., to determine changes in the recipient's microbial community and/or one or more health changes (including measuring biomarkers). The microbial community of the recipient is assessed (e.g., by nucleic acid sequencing) and compared to the microbial community of the donor. One or more strains capable of colonizing and engrafting in the recipient's microbial niche are identified. These may now be isolated from the donor's or recipient's microbial community. The strains may then be administered to the same recipient or a different recipient. After a period of time, the recipient is assessed again, e.g., to determine changes in the recipient's microbial community and/or one or more health changes (including measuring biomarkers). The microbial community of the recipient is assessed (e.g., by nucleic acid sequencing) and residency of the one or more previously identified strains is assessed. Additional strains to the ones previously identified may also be identified in this round that are successful in colonizing and engrafting. Strains my thus be optimized by sequential isolation from recipients and repeated administration to recipients and subsequent identification. Further, in vitro testing and propagation may be used in between repeated administration steps to further optimize the one or more strains that have been identified.

In other embodiments, a donor sample can be administered to a recipient more than one time, e.g., two, three, four or more times. In some embodiments, recipient receives two or more donor samples from different donors.

### Colonization and Engraftment

In some embodiments, the one or more strains identified as described herein are capable of colonizing and becoming established (engrafted) in the urogenital tract (e.g. the vaginal and or endometrial tract). In some embodiments, colonization and engraftment occurs even during menstrual discharge. The one or more strains identified as described herein may continue to reside in the urogenital tract after administration over several menstruation cycles. In some embodiments, the one or more strains identified as described herein engraft in the vaginal microbial niche over one or more than one (e.g., two, three, four, five or six) menstruation cycle upon vaginal administration. Residence time can be determined, e.g., using nucleic acid sequencing, e.g., for specific lactobacilli, e.g., comparing sequencing results prior to and post administration of the one or more strains, e.g., to determine the identity of newly added strains (e.g., one or more strains of lactic acid producing bacteria) to the recipient's microbial community, and then at predetermined time intervals (e.g., prior or post a menstruation cycle, optionally, over a number of menstruation cycles) determine (e.g., through nucleic acid sequencing) that all or a certain subset of the newly added strains are still substantially present at the specific time interval. The residence time may vary depending on various factors including hormone levels (e.g., estrogen), diet, sexual activity, acidity status of the vagina, and the presence or absence of genital infections and other microbial perturbations, e.g, treatment with antibiotics.

Successful colonization and engraftment of the one or more strains identified by the methods described herein may be indicated by one or more of: decreased pH, increased lactic acid content, lower abundance of antibiotic resistance genes, decreased amount of fungal DNA, decreased toxin content, decreased pathogenicity factors, decreased inflammatory cytokines and chemokines, decreased immune cell infiltrates, decreased total bacterial DNA load, increased viscoelasticity, increased sialoglycan content, decreased relative or absolute abundance of pathobionts or pathogens, or any combination thereof in the vaginal cavity and the vaginal microbial niche (e.g., when compared to baseline of the same subject (e.g., prior to administration and engraftment)).

### Bacterial Strain Isolation and Propagation

The methods for identifying bacterial strains capable of colonizing and engrafting the female genitourinary tract optionally further comprise one or more steps to isolate, test, propagate and formulate (e.g., dosage forms, preparations, compositions, such as, e.g., pharmaceutical compositions) the one or more strains that are identified using the methods described herein. As described herein, one or more strains capable of colonizing and engrafting the female genitourinary tract can be identified by comparing the constituents (bacterial taxa) of the donor female with those of the recipient female (e.g., by nucleic acid sequencing), after a predetermined amount of time and/or after determining a desired outcome (e.g., changes in dysbiosis or reduction in one or more disease-associated symptoms). Strains identified from the recipient sample that match one or more strains from the donor sample by a predetermined degree of sequence identity are thought to be capable of colonization and engraftment, and, in some embodiments, associated with resolution of dysbiosis and/or disease-associated health symptoms, or another desired outcome. Upon obtaining the identification of the strains that are successful in colonizing and engrafting the microbial niche in vivo, the strains so identified are then isolated from the healthy donor sample comprising the healthy donor microbial community. Alternatively, if desired, the strains so identified are then isolated from a recipient sample comprising the modulated or shifted microbial community that is associated with a reduction in dysbiosis and/or a reduction of disease-associated health symptoms, or another desired outcome.

Isolation and propagation of bacterial strains are known in the art and a variety of methods exist to accomplish bacterial isolation and propagation in vitro.

For example, the lactic acid producing bacteria (e.g., from the donor sample comprising the donor (vaginal) microbial community) or the recipient sample comprising the recipient (vaginal) microbial community) are isolated, e.g., from human vaginal fluid/cervicovaginal secretions, selected and cultured in vitro in a suitable nutrient medium providing at least one isolated bacterial strain. The at least one isolated strain is then optionally tested for its ability to colonize and engraft by administering the isolated strain to a female recipient. The at least one strain is then tested for residence time in the microbial niche of the urogenital tract of the recipient and/or its associating with one or more health-related outcomes, e.g., resolution of dysbiosis or reduction of one or more disease-associated symptoms exhibited by the female recipient prior to administration of the one or more isolated strains.

Alternatively, or in addition, the isolated strains are tested for the presence of antimicrobial resistance (AMR) genes. Preferably, the isolated strains comprise a low abundance (or are substantially free) of AMR genes. Antimicrobial resistance (AMR) genes include genes that confer resistance to one or more antibiotics, including, e.g., aminoglycosides, beta-lactams, tetracyclines, and sulfonamides (e.g., as described and cataloged in the NCBI National Database of Antibiotic Resistant Organisms (NDARO)). It will be appreciated that due to extensive use of antibiotics the cut-off is not zero. Some reasonable allowance for the presence of AMR genes is made. The precise cut-off can be determined by one of ordinary skill, based on, e.g., the nature of the AMR genes (e.g., degree of health concern) and public health recommendations.

Alternatively, or in addition, the isolated strains are tested for the presence of one or more pathogenicity factors/bacterial virulence factors, and/or colonization factors.

Further, if desired, though not generally necessary for the methods described herein, the one or more isolated strains can undergo one or more in vitro functionality tests (either prior to, concurrent with or after administration to a female recipient), e.g., to supplement or complete the data obtained from in vivo testing, including a) growth in a culture medium, b) antagonism of pathogens (bacterial, viral, and/or fungal) that inhabit the vaginal mucosa, c) high viability during fermentation, d) persistent colonization of mucosal surfaces (e.g., as judged by in vitro adherence assays, e.g., VEC), e) high lactic acid production, f) genetic stability upon repeated cultivation and in vivo upon vaginal administration, and/or g) high viability after final processing, e.g., preserved in a diluent, in lyophilized (desiccated) or (micro) encapsulated from, and storage. Each of the distinguishing characteristics described herein can be detected according to standard methods known in the art.

For example, antagonism to pathogens may be determined by measuring the production of hydrogen peroxide by the isolated strain in vitro, e.g., by colorimetric assay (e.g., using tetramethylbenzidine-containing medium or using commercially available hydrogen peroxide detection strips). Alternatively, or in addition antagonism to pathogens may be determined by measuring plating assays with the isolated strain(s) and one or more pathogens and determining the size (e.g., diameter) and number of the inhibition zones around the colonies of the isolated strain(s). For example, rapid growth can be measured, e.g., by determining the growth rate (population doubling) into late log phase or stationary phase, e.g., in comparison to a comparator strain or known growth rates of other members of the same species. For example, a high rate of viability and percent viability (e.g., after culturing or desiccation) can be measured, e.g., by determining the growth rate and/or number of dead/life cells, after exposure to stress, e.g., variations in culturing temperature, pH (e.g., low pH, high pH) richness of the growth medium, concentration of buffering agents, etc., and exposure to desiccation (e.g., freeze-drying, lyophilization) and storage (and measuring the percent viable cells at predetermined intervals). Life/dead cell counts can be performed visually, e.g., with a microscope, e.g., with a dark field condenser and a Petroff-Hausser counting chamber to obtain a total cell count, or by colony forming units (CFU) on appropriate media plates, and the Live/Dead ratio calculated as CFU per ml/total cells per ml. For example, lactic acid levels can be determined, e.g., in culture media, using a commercially available lactate assay or using common chemical separation techniques such as capillary electrophoresis and chromatography (e.g., HPLC). For example, the ability of the isolated strain(s) to adhere to the vaginal mucosa in vivo may be assessed in vitro using vaginal epithelial cells (VEC), cultured Caco-2 cells or other cell lines (e.g., HeLa) by determining an adherence value, e.g., by calculating the percent vaginal epithelial cell (VEC) cohesion value, defined as the percentage of VECs, where at least one bacterial cell is adhered to in the total number of VECs. Alternatively, the adherence is determined by calculating the percent vaginal epithelial cell (VEC) cohesion value, defined as the percentage of VECs, where at least one bacterial cell is adhered in the total number of VECs. Another measure of in vitro adherence is to count the average number of microbial cells adhered to a pre-defined number of epithelial cells. For example, the stability of the bacterial genetic profile of the isolated strain(s) upon repeated cultivation in large-scale production, and during manufacturing processes of, for instance, (pharmaceutical) compositions may be tested. In one embodiment, the isolated strain(s) preserve their genetic profile upon at least 10 repeated cultivations.

The isolated strain(s) are preferably evaluated and selected as to genetic stability in vivo upon vaginal administration, e.g., they are genetically stable in vivo for at least 1 month, 2 months, 3 months, 4 months, 5 months, 6 months, 8 months, ten months, or for at least 12 months.

The isolated strain(s) are preferably evaluated and selected as to their viability upon cryo-preservation (e.g., freezing) and storage, as well as freeze-drying/lyophilization. The isolated strain(s) are preferably viable for up to several months (e.g., 1, 2, 3, 4, 5, 6, 9, 12, 15, 18, or 24 months, or longer) if stored frozen at about −18° C., or preferably at about −80° C. Viability decreases over time. The isolated strain(s) are suitable for use if they retain at least 1%, 5%, 10%, 20%, 30%, 40%, 50%, 60%, 70%, or at least 80% viable bacteria prior to use. In some embodiments, the isolated strain(s) retain at least 30%, 40%, 50%, 60%, 70%, or at least 80% viable bacteria prior to use. Viable cells are generally able to colonize and engraft. Percent viability refers to the percentage of viable bacteria in a population. If desired, the one or more isolated strains are further processed to be freeze-dried (lyophilized) and/or pelleted, e.g., for easy storage, packaging, formulated in, for instance, compositions, and transport, and can be rehydrated before administration.

In some embodiments, the isolated strain(s) are provided in formulations suitable for prolonged stability over time. In some embodiments, the formulations comprise one or more of PVA, form gel polaxamer, form gel HA, form gel Carbopol, lyophilized gel NACMC, lyophilized gel HA, lyophilized gel guar gum, tablet NACMC (3 tons), tablet NACMC (1 ton), tablet PVP, or tablet polaxamer.

The bacterial strains capable of colonizing and engrafting the female genitourinary tract, e.g., isolated by the methods described herein can be propagated by any means know in the art. For example, lactic acid producing strains (including, e.g., Lactobacillus strains) can be grown in a number of different types of media, in either liquid, semisolid (e.g., agar), or solid form. The culturing conditions can be similar to the natural environment inhabited by the bacteria.

Standard bacterial media generally include salts, a source of carbohydrate, and a pH buffer. For Lactobacillus, pH is generally maintained in the range of about 4.5 to about 7.0, preferably between pH 5,5-6,5 with the addition of sodium phosphate, arginine, ammonium hydroxide, sodium hydroxide, potassium hydroxide, etc. Vitamins and growth agents, including amino acid formulations, can also be added. Bacterial media are known and commercially available (e.g., from Sigma-Aldrich), including, e.g., MRS (including Lactobacillus-MRS Agar (De Man, J. et al., J. Appl. Bact. 23:130-135, 1960)), Rogosa, NZM, YM, M17, Thayer-Martin, Trypticase Soy, CDM (Geshnizgani A. M., and Onderdonk A. B., (J Clin Microbiol. 1992 May; 30 (5): 1323-6)), and Brain-Heart infusion (BHI) broth. Carbohydrates useful for growing the present strain include D-galactose, D-glucose, D-fructose, D-mannose, D-mannitol, N-acetylglucosamine, amygdalin, arbutin, esculin, salicin, D-cellobiose, D-maltose, sucrose, D-trehalose, amidon, and glycogen. Some of the media can be made selective for lactic acid producing bacteria, such as lactobacilli, e.g., by using a low pH, and addition of certain additives, e.g., sodium acetate and ammonium citrate, e.g., to inhibit other flora and fungi. In some embodiments, the medium comprises Brucella blood agar with hemin and vitamin K, which enables growth of a wide range of vaginal bacteria.

Lactobacilli are aerotolerant organisms highly directed towards fermentation. Lactobacilli naturally colonize mammalian mucosal surfaces, and generally grow optimally at about body temperature, e.g., 37° C., though they also grow at lower (e.g., 30° C.) and higher temperatures (e.g., 40° C.). The growth rate can be controlled by changing the temperature.

After reaching the desired bacterial cell density, the bacterial cells can be harvested using any suitable method to remove the cells from the culture medium. Non-limiting exemplary methods for harvesting the cultured cells include filtration, centrifugation, and sedimentation. In some embodiments, the cell biomass is washed at least once using a physiologically buffered solution. In some embodiments, the wash solution may contain additional components, such as, e.g., glucose. In some embodiments, the wash solution comprises a buffer or one or more buffering agents. Exemplary buffering agents that may be used or added to the wash solution include phosphate salts, (e.g., Na₂HPO₄, NaH₂PO₄, NaHCO₃, and arginine).

Formulation into Preparations and Pharmaceutical Compositions

The one or more strains capable of colonizing and engrafting identified by the methods described herein can be formulated into strain preparations and (pharmaceutical) compositions and various dosage forms, e.g., using one or more carriers, excipients, diluents and/or buffers, that can optionally, be pharmaceutically acceptable. In some embodiments, the dosage form can be an applicator or dispenser, e.g., those commonly used in medicine and science. In some embodiments, the dosage form can be a vaginal suppository that will remain in the vaginal cavity until it is dissolved.

Other dosage forms suitable for formulation include a suspension, spray, gel, cream, powder, (gelatin or vegetable cellulose) capsule, solution for lavages, douches, ovules, a vaginal insert, tablets, disk, wafer (e.g., drying on film, by vaporization), or a microencapsulated product employing excipients and formulation techniques known to those skilled in the art. Suitable dosage forms are formulated to readily dissolve and/or disperse inside the vaginal cavity.

Other dosage forms suitable for formulation include absorbent products comprising the bacterial composition, such as, e.g., a feminine hygiene diaper, panty liners, sanitary napkin, tampon, panty guard or an incontinence guard.

The one or more strains capable of colonizing and engrafting identified by the methods described herein can be formulated in dry form, e.g., lyophilized or spray dried and then optionally formulated into a dosage form described herein, or reconstituted, e.g., with sterile water, a weak acidic solution, gel, or buffer prior to use. If the composition is not reconstituted, depending on the precise formulation of the dosage form, rehydration may also be achieved inside the vaginal cavity, e.g., aided by resident vaginal fluid. Methods of preserving viable bacteria by lyophilization can promote long-term preservation of the microorganism. One skilled in the art will be able to lyophilize bacteria using standard techniques.

In all of these embodiments, dyes, perfumes, pH buffering agents, drying or resuspending agents, or other standard materials for drug formulation can be incorporated into the compositions and dosage forms.

In all of these embodiments, devices and compositions described herein may be packed in a suitable packaging, for example in a bottle, flacon, blister pack, cartridge, applicator or dispenser.

### Methods to Define Profiles, Including, Microbial, Dysbiotic, Health, Reference, Donor, and Recipient Profiles

Further provided herein are methods of defining vaginal microbial communities, such as donor and/or recipient vaginal microbial communities, e.g., to increase the success rate of colonizing the recipient's female genitourinary tract with one or more strains capable of colonizing the human female genitourinary tract identified by the methods described herein.

The methods include defining profiles, e.g., by means of data, such as nucleic acid sequencing data, e.g., derived from the donor and/or recipient vaginal microbial communities (microbial profile), e.g., a profile from the vaginal microbial communities, e.g., a pangenome or supragenome of selected microorganisms; and, if desired, health and other subject data, e.g., health history, age and/or hormonal status, race/ethnicity, other genetic traits, and immune/inflammatory marker profile (health profile). For example, for genetic data, a profile may include, e.g., nucleic acid profiles (e.g., sequences) that are enriched (or highly amplified), and/or profiles may comprise a totality of inputs (e.g., all genes or genomes from all strains within a predefined group, e.g., a clade, such as, e.g., a pangenome). For health data, a profile may include a comorbidity that is highly common among (e.g., enriched in) a test group, and/or a profile may consist of the totality of all ages (e.g., represent a range). In embodiments, the profile is selected at least in part based on identifying at least one genetic element associated with the capability to successfully engraft/colonize the female genitourinary tract of a female recipient or likelihood thereof. In embodiments, the profile may thus act as a guiding template to select microbial strains as described herein.

For example, a recipient profile may comprise: (1) a microbial profile, e.g., nucleic acid sequencing data, e.g., derived from the recipient vaginal microbial community in its current state, e.g., prior to administration of one or more strains capable of colonizing and engrafting in the urogenital tract; the recipient may exhibit a dysbiosis (e.g., associated with inflammation and/or an infection, e.g., vulvovaginal candidiasis, bacterial vaginosis); and/or (2) a health profile that includes one or more of the following information and data: recipient health history (including chronic conditions, e.g., obesity, diabetes; sexually transmitted disease, smoking, alcohol intake, drug use, etc.), current medications, underlying indication (e.g., disease/disorder), age and/or hormonal status (including pre-, post-menopausal, pregnancy, puberty), race/ethnicity (e.g., Caucasian, Black/African, Hispanic, Asian, etc.), other genetic traits, Body Mass Index (BMI), and/or immune/inflammatory profile (e.g., inflammatory biomarkers).

A profile, e.g., a recipient profile (or a donor profile) may further comprise data derived from one or more host (subject) biomarker(s) and/or microbiota-associated biomarker(s), systemic or local, such as, e.g., metabolomics, cytokine panels, host genetic markers, and host hormonal markers. The profiles can be generated, e.g., by aggregating data over a plurality (e.g., 5, 10, 20, 30, 40, 50, 100, 200, 300, 500, 1000) of subjects (e.g., donor or recipient) and associated microbial samples (e.g., vaginal microbial samples, e.g., derived from vaginal fluid/vaginal secretions). Data aggregation techniques are known to those in the art and may include various calculations, e.g., of means, averages, deviations, derivatives, etc.

The profiles once generated can be provided to one or more users, e.g., physicians, clinicians, other healthcare providers, or a home user (e.g., a self-treating subject, or a subject being treated by family or other household members), e.g., to inform healthcare decisions, such as, e.g., appropriate treatment of a certain condition presented by a female subject. In one embodiment, the profile is used to make decisions concerning standard of care. In one embodiment, the profile is used to make decisions concerning the selection of isolated strains capable of colonizing and engrafting the urogenital tract (e.g., selecting strain(s) with the highest probability of colonization and engraftment), e.g., to treat a disease or disorder associated with the urogenital tract (including treating vulvovaginal candidiasis and dysbiosis).

Provided herein are methods of defining vaginal microbial donor communities, such as, e.g., a donor profile. The methods include: obtaining a plurality of vaginal donor samples, wherein each donor sample comprises vaginal microbes obtained from a different non-dysbiotic (e.g., healthy) donor female (e.g., from vaginal fluid, e.g., a cervicovaginal secretion); using an analytical technique to obtain a plurality of vaginal microbial profiles from the plurality of vaginal samples; identifying a profile from among the plurality of microbial profiles, thereby defining a plurality of vaginal microbial donor communities.

Optionally, the microbial profile can be combined with a health profile, that includes one or more of the following information/data: donor health history (including chronic conditions, e.g., obesity, diabetes; sexually transmitted disease, smoking, alcohol intake, drug use, etc.), current medications, existence of underlying conditions (e.g., disease/disorder), age and/or hormonal status (including pre-, post-menopausal, pregnancy, puberty, and being on hormonal contraception), race/ethnicity (e.g., Caucasian, Black/African, Hispanic, Asian, etc.), other genetic traits, Body Mass Index (BMI), and/or immune/inflammatory profile (e.g., inflammatory biomarkers).

Provided herein are methods of defining vaginal microbial recipient communities, such as, e.g., a recipient profile. The methods include: obtaining a plurality of vaginal recipient samples, wherein each recipient sample comprises vaginal microbes obtained from a different recipient female (e.g., from vaginal fluid, e.g., a cervicovaginal secretion); using an analytical technique to obtain a plurality of vaginal microbial profiles from the plurality of vaginal samples; identifying a profile from among the plurality of microbial profiles, thereby defining a plurality of vaginal microbial recipient communities.

For example, where the recipient presents with dysbiosis, the methods include: obtaining a plurality of vaginal samples, wherein each sample comprises vaginal microbes obtained from a different dysbiotic female (e.g., from vaginal fluid, e.g., a cervicovaginal secretion), using an analytical technique to obtain a plurality of vaginal microbial profiles from the plurality of vaginal samples, identifying a dysbiotic profile from among the plurality of microbial profiles, thereby defining a plurality of vaginal microbial recipient communities.

Optionally, the microbial profile can be combined with a health profile, that includes one or more of the following information/data: recipient health history (including chronic conditions, e.g., obesity, diabetes; sexually transmitted disease, smoking, alcohol intake, drug use, etc.), current medications, existence of underlying conditions (e.g., disease/disorder), age and/or hormonal status (including pre-, post-menopausal, pregnancy, puberty, and being on hormonal contraception), race/ethnicity (e.g., Caucasian, Black/African, Hispanic, Asian, etc.), other genetic traits, Body Mass Index (BMI), and/or immune/inflammatory profile (e.g., inflammatory biomarkers).

Typically, a recipient will exhibit one or more of: a microbial dysbiosis, e.g., a dysbiosis of the urogenital tract, a disease or disorder, e.g., associated with (chronic) inflammation, and/or an infection (e.g., bacterial vaginosis). A recipient may present with a wide variety of other conditions including one or more of: chronic conditions, e.g., obesity, diabetes; sexually transmitted disease, smoking, alcohol intake, drug use, intake of current medications, old age, young age (including pre-, post-menopausal, pregnancy, puberty), and/or chronic inflammation.

In one embodiment, the methods include determining from the recipient (e.g., dysbiotic) profiles a suitable donor profile for administration of a vaginal microbial donor community to a recipient female in need thereof.

In one embodiment, the methods include determining from the recipient (e.g., dysbiotic) profiles a suitable treatment regimen (e.g., including standard of care treatments, e.g., antimicrobial, anti-inflammatory, or hormonal treatments) for the condition presented by the recipient female. Optionally, the subject is informed of their profiles. In some embodiments, the profiles provide valuable diagnostic guidelines for the evaluation of subjects with respect to health and disease status.

In one embodiment, the methods include providing or obtaining a vaginal fluid/vaginal secretion sample from a subject (e.g., a donor female or a recipient female), conducting an assay to identify (and/or quantity, e.g., abundance of) a plurality of microbes present in said sample (e.g., genus, species or strain), optionally processing the data comprising the identity of the plurality of microbes present in said sample, e.g., in order to obtain a subset of identities of the microbes, to provide a profile (e.g., a microbial profile); comparing the microbial profile to a reference profile (e.g., a profile associated with dysbiosis and/or inflammation, or a disease or disorder, e.g., one that is associated with the urogenital tract), and if there is a substantial match (e.g., based on one or more predetermined parameters) between the microbial profile and the reference profile, informing a healthcare provider or a subject of their health status and/or a suitable treatment regimen. Suitable assays for these methods include those described herein, including, nucleic acid sequencing. If desired, the methods include comparing a health profile (e.g., the subject's health profile with a reference health profile, e.g., from a reference population), thereby informing a healthcare provider or a subject. In some embodiments, the methods comprise assessing potential success with colonization and engraftment, e.g., of one or more strains capable of colonizing and engrafting in the female urogenital tract described herein.

The methods may further comprise obtaining profiles (e.g., microbial profiles), e.g., derived from a vaginal microbial sample, assaying the sample to determine the presence, abundance (e.g., overall microbial burden), and/or diversity of microbes, and comparing the results to a reference profile having known associations with successful colonization and engraftment. In some embodiments, the reference profile is determined at different time points (e.g., menstrual cycle, age, hormonal state, health/disease state, dysbiotic state, inflammatory state, etc.) and/or across a plurality of subjects (e.g., a reference population). By ascertaining the microbial profile from numerous samples with a shared characteristic, profiles can be determined that provide a reference point for further analysis.

A reference profile can be, e.g., obtained from the microbial community of normal vaginal microbiota, e.g, a plurality of vaginal microbiota profiles is obtained from females without a vaginal pathology (healthy female. One or more profiles is identified among the plurality of vaginal microbiota profiles; and at least a subset of the plurality of vaginal microbiota profiles is assigned to a profile that defines normal vaginal microbiota. Alternatively, or in addition, reference profile can be, e.g., obtained from the microbial community of normal vaginal microbiota, e.g., a plurality of vaginal microbiota profiles is obtained from females with abnormal (e.g., dysbiotic) vaginal microbiota, e.g., reference profiles based on microbial populations present in the urogenital tract of women with symptoms of a vaginal pathology.

A reference profile can be, e.g, obtained by sampling during routine gynecological exam, e.g., to provide a baseline reference, e.g., to establish a distribution of vaginal microbiota that is normal for a particular woman (normal vaginal ecology). Such a baseline profile provides a convenient comparison in the event the subject presents with symptoms of a condition or disease affecting vaginal health.

Normal vaginal microbiota varies among women, and between women of different racial and/or ethnic backgrounds. Multiple profiles (e.g., donor, recipient profiles) may be identified that define categories of normal, and conversely, categories of abnormal, vaginal microbiota in a plurality of women, regardless of how the pluralities are defined, i.e., how the distinctions between different pluralities of women are drawn (e.g., along criteria such as, health condition, age, racial and/or ethnic ancestry, etc.).

In another example, microbial profiles, e.g., assigned to a subject exhibiting a disease or condition associated with the urogenital tract, can be compared to a reference profile (e.g., a profile generated from a plurality of women) to provide an indication of the subject's potential for successful engraftment of one or more isolated strains that are associated with colonization and a health benefit (e.g., identified by the methods described herein). An evaluation may be performed based on one or more predetermined parameters. The evaluation may include, selecting a specific set or subset of isolated strain(s) that have a high probability of successful engraftment and/or informing, e.g., the healthcare provider of a treatment regimen that includes one or more co-treatments (e.g., antimicrobial, anti-inflammatory, or hormonal treatment), or providing other steps to prepare the urogenital tract for administration of the strains or maintenance of the new microbial community after administration (e.g., providing acidification, prebiotics, etc.).

In some embodiments, the microbial profiles comprise data (e.g., nucleic acid sequences) identifying additional lactic acid producing bacteria. In some embodiments, the microbial profiles comprise data (e.g., nucleic acid sequences) identifying one or more strains belonging to one or more species selected from Lactobacillus crispatus, Lactobacillus iners, Lactobacillus jensenii, and Lactobacillus gasseri.

Typically, the microbial profiles are provided by a culture-independent method. For example, the microbial profiles can be provided by preparing a nucleic acid sample directly from (vaginal) microbial samples (e.g., vaginal fluid/vaginal secretion). In some embodiments, taxa (genus, species, strains) may be identified using polymorphic polynucleotides, such as a phylogenetically informative gene. The identity can be determined, for example, by determining the nucleotide sequence of the polymorphic polynucleotide, or a portion or subsequence thereof. Phylogenetically informative genes include functional genomic sequences, such as, protein coding regions and/or regulatory regions. Phylogenetically informative genes (e.g., homologs or orthologs of a gene) differ between species but originate from a common ancestor. The polynucleotide sequences of orthologous genes in different species have diverged over time accumulating mutations, that is, nucleotide alterations (which can be insertions, deletions, point mutations, and/or recombination events), which can be detected using any of a variety of methods for detecting sequence differences. Typically, a phylogenetically informative gene is one for which at least one ortholog can be detected among a large number of species of microorganisms. For example, a conserved gene, such as a house-hold gene is used, e.g., that is found in a large number of species and then sequence variations in that gene are determined.

Microbial profiles can be differentiated using any of a variety of methods, including, e.g., clustering algorithms and other methodologies. In general, clustering is a type of unsupervised learning in which the classes are unknown a priori and the goal is to discover these classes from data. For example, a variety of donor and recipient classes can be identified. In some embodiments, identification of classes aids the matching of donors and recipients.

Additional methods and models useful for defining and interpreting one or more profiles (e.g., microbial, health, dysbiotic, reference, donor, or recipient profiles described herein) include logistic regression, ordinal logistic regression, linear or quadratic discriminant analysis, clustering, principal component analysis, nearest neighbor classifier analysis, and discrete time-proportional hazards models.

Logistic regression analysis may be used to generate an odds ratio and relative risk for each characteristic. Linear discriminant analysis attempts to classify a subject into one of two categories based on certain object properties. Quadratic discriminant analysis takes the same input parameters as linear discriminant analysis but uses quadratic equations, rather than linear equations, to produce results.

Microbial profiles can be generated by methods known in the art. In some embodiments, data are used to cluster a training set Clustering is described, e.g, in Duda and Hart, Pattern Classification and Scene Analysis, 1973, John Wiley & Sons, Inc., New York: Kaufman and Rousseeuw, 1990, Finding Groups in Data: An Introduction to Cluster Analysis, Wiley, New York, N.Y.; Duda, Pattern Classification, Second Edition, 2001, John Wiley & Sons, Inc; and Hastie, 2001, The Elements of Statistical Learning, Springer, New York; Everitt, 1993, Cluster analysis (3rd ed.), Wiley, New York, N. Y.; and Backer, 1995, Computer-Assisted Reasoning in Cluster Analysis, Prentice Hall, Upper Saddle River, N.J., Clustering algorithms or other statistical models include the Cubical Clustering Criterion (CCC, Sarle The Cubic Clustering Criterion. SAS Institute (1983), SAS Technical Report A-108, SAS Institute Inc., Cary N. C. (1983)); pseudo F index (Calinski & Harbasz (Communications in Statistics 3:1-27 (1974)); and pseudo T2 test (Duda & Hart Pattern Classification and Scene Analysis John Wiley & Sons, Inc., New York (1973)), or a combination thereof.

For example, clustering involves (a) defining a suitable distance between objects, and (b) selecting and applying a clustering algorithm, such as, e.g., hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Alternatively, or in addition, classification methods (or class prediction) can be utilized, e.g., described in Duda, Pattern Classification, Second Edition, 2001, John Wiley & Sons, Inc. Classification methods include principal component analysis, discriminant analysis (linear and quadratic discriminant), nearest neighbor classifiers (k-nearest neighbor), classification and regression trees (CART), multivariate decision trees, ID3, and C4.5, evolutionary algorithms, neural networks and multinomial log-linear models, support vector machines, and aggregated classifiers (bagging, boosting, forests). Other models include multiple additive regression tree (MART) models and Cox proportional hazards model (Cox, David R (1972). “Regression Models and Life-Tables”. Journal of the Royal Statistical Society, Series B. 34 (2): 187-220).

Methods useful for constructing clusters include, e.g., pair-wise distances method, maximum variation method, systematic cover method, and cover sampling method (e.g., described in U.S. Pat. No. 7,627,437 Categorization of microbial communities). The selection of methods depends on the amount of variation in the cluster. The lowest resolution results from using the systematic cover method, which focuses on richness alone in choosing a sample. The highest resolution results from choosing a sample using the maximum variation method, which is directed at explaining as much of the variation in the cluster as possible. The pair-wise distances method and the cover sampling method provide intermediate resolutions. Following cluster analysis, the samples may optionally be further analyzed to identify profiles.

These, and other methods known in the art are suitable for determining profiles (e.g., microbial, health, dysbiotic, reference, donor, or recipient profiles described herein) and performing comparisons between profiles.

### Definitions

The definitions hereinafter apply to all aspects and embodiments disclosed herein.

### General

Unless otherwise explained, all technical and scientific terms used herein have the same meaning as commonly understood by one of ordinary skill in the art to which this disclosure belongs. It is further to be understood that methods and materials similar or equivalent to those described herein can be used in the practice or testing of this disclosure. The materials, methods, and examples are illustrative only and not intended to be limiting. Other features of the disclosure are apparent from the following detailed description and the claims.

The singular terms “a,” “an,” and “the” include plural references unless context clearly indicates otherwise. Similarly, the word “or” is intended to include “and” unless the context clearly indicates otherwise. The term “comprises” means “includes.” The abbreviation, “e.g.” is derived from the Latin exempli gratia, and is used herein to indicate a non-limiting example. Thus, the abbreviation “e.g.” is synonymous with the term “for example.” The term “optional” or “optionally” means that the subsequent described event, circumstance or substituent may or may not occur, and that the description includes instances where the event or circumstance occurs and instances where it does not.

Unless otherwise indicated, all numbers expressing quantities of components, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art.

All patents, patent applications, and publications identified are expressly incorporated herein by reference for the purpose of describing and disclosing, for example, the methodologies described in such publications that might be used in connection with the present invention

In a preferred embodiment, the term “about” shall allow a deviation of +10%, and even more preferably of +5% from an indicated numerical value.

The term “comprising” shall be understood to simultaneously also disclose the term “consisting” as a preferred option. For example, if a composition is said to comprise three components, this also discloses a composition consisting of these three components as preferred embodiment.

If the term “comprising” is used when referring to (a) pharmaceutically active compound(s), bacterium (a), and the like, this shall be understood to simultaneously also disclose that the pharmaceutically active compound(s), bacterium (a), and the like is/are preferably the sole pharmaceutically active compound(s), bacterium (a), and the like. For example, if a donor sample is said to comprise Lactobacillus crispatus and Lactobacillus iners, this simultaneously and preferably discloses that a donor sample contains Lactobacillus crispatus and Lactobacillus iners as the sole bacteria. If a donor sample is said to comprise Lactobacillus crispatus, Lactobacillus iners, and an excipient, this simultaneously and preferably thus discloses that a donor sample contains Lactobacillus crispatus and Lactobacillus iners as the sole bacteria and in addition comprises an excipient. It further discloses, that the donor sample consists of Lactobacillus crispatus, Lactobacillus iners, and an excipient.

As used herein, a “nucleic acid” includes DNA and RNA (e.g., mRNA, IRNA, or tRNA) and analogs thereof, including nucleic acids comprising conventional nitrogenous bases (e.g., A, G, C, T. U), base analogs (e.g., inosine), derivatives of purine or pyrimidine bases and any substitutions

As used herein, the term “isolate nucleic acids” refers to the purification of nucleic acids from one or more cellular components. Isolated nucleic acids can be prepared from specimens using any acceptable method known in the art. For example, cells can be lysed using known lysis agents, and nucleic acids can be purified or partially purified from other cellular components.

As used herein, “sequence identity” or “percent identical” as applied to nucleic acid molecules is the percentage of nucleic acid residues in a candidate nucleic acid molecule sequence that are identical with a subject nucleic acid molecule sequence, after aligning the sequences to achieve the maximum percent identity, and not considering any nucleic acid residue substitutions as part of the sequence identity. Nucleic acid sequence identity can be determined using any method known in the art, for example CLUSTALW, T-COFFEE, BLASTN.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least about 95% identity to a given reference sequence Percent identity can be anywhere from 90% to 100%. Most embodiments include at least: 95%, 96%, 97%, 98%, 99% or 99.9% compared to a reference sequence using the programs described herein (e.g., BLAST, using standard parameters).

A “microbial profile” is a set of the species and/or strains of microorganisms present in a sample of microorganisms. To the extent that a sample of microorganisms is obtained from, and corresponds to the species found in, a shared environment, the microbial profile details the species present in a microbial community. The term “profile” as used herein refers to both an individual profile and an aggregate profile over a plurality of individual profiles, depending on the context of its use. In some embodiments, an aggregate profile may include, e.g., a pangenome (sequence), e.g., all the genes (or genomes) of all individual members (e.g., strains) within a particular grouping (e.g., clade).

As used herein the term “lactic acid producing bacteria” means bacteria that produce lactic acid by fermentation.

As used herein the term “bacterial sample” or “microbial sample” means a sample comprising bacteria. The sample can be, e.g., vaginal fluid/discharge.

As used herein the term “suitable nutrient medium” means a medium in which the bacteria might be cultivated.

“Genitourinary tract” or “urogenital tract” as used herein as used herein includes the uterus, fallopian tubes, ovaries, vagina, cervix, vulva, and urinary tract. In some instances, used herein are teachings and exemplifications specifically calling out the “vaginal tract”, “vaginal cavity” or “vagina”. One of ordinary skill will appreciate that these exemplifications and teachings are illustrative only and non-limiting and, thus also apply, where appropriate, to other anatomical sites of the genitourinary tract or urogenital tract, not just to the vagina.

“Dysbiosis” as used herein means a microbial imbalance, i.e. an aberration of the healthy state, where normally predominant species are diminished in abundance and less predominant species become more abundant and/or predominant. Vaginal dysbiosis is a microbial imbalance in the vagina, wherein the vaginal imbalance may, in embodiments, extend to the endometrium. Dysbiosis is generally associated with one or more of: (a) qualitative and quantitative changes in the content or amount of the microbiota itself, (b) changes in their metabolic activities; and/or (c) changes in their local distribution (e.g., inside a niche). A dysbiotic human microbiota (or microbiome) refers to a population of microbes that promotes inflammation of a tissue of the urogenital tract (e.g, the vagina) and/or that contributes to or establishes an environment that permits or promotes the colonization or growth of one or more pathogenic microbes. Dysbiosis also refers to a perturbation of the urogenital (e.g., vaginal) homeostasis. In some embodiments, a dysbiotic vaginal microbiota will generally result in increased pH relative to a healthy microbiota, e.g., a pH above 4.5, e.g., a pH of 5.0, 5.5, 6.0, 6.5, 7.0 or higher. In some embodiments, vaginal dysbiosis is characterized by a reduction of Lactobacillus spp., and an increased diversity of vaginal anaerobic bacteria. In some embodiments, vaginal dysbiosis is associated with upper genital tract infections or pelvic inflammatory disease (PDI), and increased risk of sexual transmitted diseases. Dysbiosis may be characterized by the relative amount of selected pathogens, such as >20% selected pathogens, and the relative low abundance of vaginal lactobacilli, e.g. <10% vaginal lactobacilli (L. crispatus, L. iners, L. jensenii, L. gasseri). Dysbiotic subjects as used herein comprise subjects having vaginal symptoms (symptomatic subjects) and subjects not having any vaginal symptoms (asymptomatic subjects), wherein symptoms are characterized by Amsel's criteria, Nugent score and/or Hay/Ison score.

A “desired change” of dysbiosis may thus comprise prophylaxis and/or treatment of any of the above mentioned symptoms associated with dysbiosis, e.g. reduction of inflammation or infection of the urogenital tract, or the restoration and/or rebalancing of the vaginal microbiota (e.g., to achieve homeostasis). The desired change of dysbiosis may alleviate at least one or more symptoms, delay the development of a symptom, alter the course of a symptom (e.g., slowing the progression of a symptom), or reverse a symptom, wherein the symptom may include an increase of beneficial Lactobacillus spp., a reduction of upper genital tract infections or pelvic inflammatory disease (PDI), a reduction of the relative amount of selected pathogens. In some embodiments, the desired change includes a decrease of selected vaginal pathogens to less than 40%, less than 30% or less than 20% and/or the increase of the relative amount of vaginal lactobacilli to at least 50%, 60%&, 70%, or 80% (L. crispatus, L. iners, L. jensenii, L. gasseri).

In contrast, the term “normal” or “healthy” “vaginal flora” or “vaginal niche” “microbiota” “urogenital tract” or “community state” or similar terms connote that a woman has no vaginal complaints and does not exhibit a vaginal pathology (e.g., no sign or symptom corresponding to or resulting from a pathology of the vagina), and the condition of the vagina is such of a relatively low susceptibility to sexually transmitted diseases and pathogens, and generally of low pH, e.g., less than or equal to pH 4.5, e.g., between 3.2 and 4.5; and generally dominated by lactic acid producing bacteria (e.g., Lactobacillus spp.). Normal vaginal microbiota or normal flora are a community of microorganisms that localize to the vagina in a normal, healthy, that is, a non-pathological, non-pathogenic and/or non-dysbiotic, state.

“Cervicovaginal secretions” or “vaginal fluid” refers to the mixture of mucus secreted by the cervix, shed epithelial cells, vaginal transudate, and bacteria found in the vagina of a woman.

“Endometrial fluid” refers to a fluid accumulation within the endometrial cavity that contains bacterial cells, host cells and microbe and host derived proteins, nucleotides and metabolites. Typically, the endometrial fluid is obtained by aspiration for further analysis.

“Isolated bacterial strain” means a strain that has been separated from other strains (e.g., from a vaginal bacterial community, e.g., derived from a sample of cervicovaginal secretions or vaginal fluid) and cultivated in vitro in a culture comprising said strain. An isolated bacterial strain is substantially free of contaminants or components that accompany the material it was derived from in its native state (e.g., such as, vaginal mucus and epithelial cells). The isolated bacterial strain is used interchangeably with the term “isolate”.

“Culture-independent method” means methods not involving isolation and/or in vitro propagation of bacterial strains, e.g., in cultures.

The term “microbe” is used synonymously with the term “microorganism” and includes bacteria (Archaea, Eubacteria), yeast, fungi, and viruses. The term “species” is used herein to refer to a taxonomically and/or genetically distinct group of microorganisms. Species may include one or more distinguishable (e.g, by sequencing) strains.

The term “microbiota” refers to a community of microorganism localized to a distinct shared environment (a “microbial niche”). For example, “vaginal microbiota” is a community of one or more species of microorganisms that are localized to, or found in, a vagina. The term “microflora” or “flora” is used synonymously with the term “microbiota.” Healthy or normal microbiota denominates the community of commensal microorganisms that colonize (inhabit) a particular microbial niche of the host, such as the vagina. Bacteria are the most numerous microbial components of the normal flora.

“Microbiome” refers to the totality of microbes (bacteria, archaea, yeast, fungi and phages) and their genomes.

“Mucosa” as used herein indicates a mucous membrane. Mucus is a secretion produced by, and covering, mucous membranes. Mucous fluid is viscous and typically produced from mucous cells (e.g., goblet cells) found in mucous membranes and submucosal glands, and rich in antiseptic enzymes (such as lysozyme), immunoglobulins, inorganic salts, proteins such as lactoferrin, and glycoproteins (mucins). Mucosal surfaces include epithelial linings of the reproductive tract (vagina) and, e.g., lactobacilli are capable of colonizing the vaginal mucosal surfaces.

As used herein, the term “effective amount” means the amount (e.g., of a SCVMP) to be administered to a typical subject (e.g., a female recipient) that is sufficient to lead to a desired beneficial or therapeutic effect in the subject. The desired beneficial or therapeutic effect includes prophylaxis and/or treatment, e.g., of dysbiosis, inflammation or an infection or urogenital tract, and also includes the restoration and/or rebalancing of the vaginal microbiota (e.g., to achieve homeostasis), e.g., an anti-inflammatory and/or anti-pathogenic state of the urogenital tract and the vaginal microbiota. An effective amount, for example, is the amount sufficient (e.g., at dosages and for periods of time necessary) to alleviate at least one or more symptom, delay the development of a symptom, alter the course of a symptom (e.g., slowing the progression of a symptom), or reverse a symptom. Effective amounts generally cause statistically significant, measurable changes.

A “lyophilized”, “spray-dried” or “freeze-dried” composition refers to a composition from which moisture has been removed, e.g., for easy storage and transport. Such compositions can be rehydrated before use (e.g., administration). The lyophilized, spray-dried, or freeze-dried composition may be further pelleted or packaged for easier storage and transportation.

As used herein, the term “viable” refers to a cell (e.g., a bacterial cell) that is able to survive in a given condition (e.g., storage for a certain period of time under particular storage conditions, e.g., including, temperature, humidity) and is generally able to colonize and reproduce (e.g., in the urogenital tract) after exposure to the condition. Percent viability refers to the percentage of viable cells in a population. For example, percent viability can refer to the percentage of lactobacilli in a pharmaceutical composition that will survive (e.g., refrigeration, freezing and/or storage) and colonize upon application to a vaginal mucosal surface. Viability can be evaluated by, e.g., CFU or VCC methods. In the CFU method, dilutions series of the samples are plated on suitable medium for the microorganisms of interest, after which the number of colonies is counted after an outgrowth period at suitable temperature. In the VCC method, samples are stained with a live/dead stain and counted in e.g., a counting chamber. The live/dead stain can be a fluorescent dye that stains cells with a compromised membrane (and hence decreased or no viability) red, and intact (live) cells green.

The abbreviation “cfu” means “colony-forming unit”.

The abbreviation “VCC” means viable cell count (as determined by life cell staining).

The phrases “excipient” “pharmaceutically acceptable carrier” “diluent” or “buffer” as used herein mean a non-active, pharmaceutically acceptable material, ingredient, composition or vehicle that is added to form part of the final formulation and/or maintains a drug or other agent in a form for delivery to a subject. Each carrier preferably is compatible with the other ingredients of the formulation, for example the carrier does not decrease the impact of an active ingredient or agent upon the treatment, e.g., the carrier is pharmaceutically inert. In some embodiments a pharmaceutically acceptable carrier can be a carrier other than water (including, e.g., a cream, emulsion, gel, liposome, nanoparticle, film, ointment and/or vaginal device). In some embodiments, a pharmaceutical composition is provided comprising the substantially complete vaginal microbiota preparations together with a pharmaceutically acceptable carrier and/or diluent (e.g., saline). These compositions allow the easy administration of the preparations by means known to the person skilled in the art. In some embodiments, a buffering agent is added, e.g., a weak acid or base that maintains the acidity at a chosen level (e.g., between pH 3.5 and 4.5) and prevents a rapid change in acidity.

The terms “contacting”, “administering” or “subjecting” and more specifically, “vaginal application” or “vaginal administration”, are used interchangeably herein and relate to a subject (e.g., a female recipient) or a specific organ or other physiological site (e.g., the urogenital tract or vaginal cavity or a subpart thereof, e.g., to a site on a vaginal wall (mucosal or endometrial surfaces) or vaginal fornices) and a dosage form, preparation or (pharmaceutical) composition that is provided or given to the subject or site, e.g., for the purpose of colonizing and engrafting a desired bacterial community. In one embodiment, administering is performed locally. In one embodiment, administering is performed topically (e.g., vaginally).

The terms “dose” and “dosage” are used interchangeably herein. A dose refers to the amount of active ingredient given to an individual at each administration. The dose will vary depending on a number of factors, including frequency of administration (e.g., daily, one or more times per week, per month, or per 3 months); size and tolerance of the individual; severity of the condition, intended result (e.g., treatment, prophylaxis, modulation or restoration of the microbial community), and the route of administration. A baseline dose can be administered and modified based on the initial response of the subject. For example, a single dose of the compositions comprising the substantially complete vaginal microbiota preparations described herein can be in the range of 10⁴ to 10¹⁰ colony forming units (per dose). In other embodiments, a single dose of the composition can be in the range of 10³ to 10¹², 10⁴ to 10⁹, 10⁵ to 10⁹, 10⁵ to 10⁸, 10⁶ to 10⁹, 10⁷ to 10⁹, or 10⁷ to 10¹⁰ colony forming units (per dose).

A “dosage form” refers to a particular physical form of a composition or preparation and depends, e.g., on the desired amount of the material to be administered and on the route of administration, e.g., oral or topical (e.g., vaginal). For example, a dosage form can be in a suppository, a tablet, a capsule, a film, a cream, etc., or a device, such as, e.g., an applicator or dispenser, e.g., for vaginal administration. Dosage forms may be single or multiple-use dosage forms.

As used herein, the term “colonization” or “engraftment” refers to the colonization of an environment (e.g., a microbial niche), e.g., the vagina or vaginal epithelium, by a microbe, e.g., a bacterium (e.g, lactobacilli), such that the viable population of that microbe continues to reside, e.g., in the niche, for a certain period of time. Engraftment can be transient or stable depending on the period of time the microbe continues to reside in the niche. Colonization and engraftment (and residence time) can be quantified, e.g., by counting the number of colony forming units (CFU)/gram and/or performing nucleic acid sequencing of microbes comprised in one or more vaginal samples that are taken over a certain period of time.

The term “subject” refers to a human (e.g., a human female). In one embodiment, the subject is 18 years or older. In one embodiment, the subject is in puberty. In one embodiment, the subject is pregnant. In one embodiment, the subject is of childbearing age. In one embodiment, the subject is pre-menopausal. In one embodiment, the subject is post-menopausal. In one embodiment, the subject is a donor female providing a microbial sample. In some embodiments, a subject is a human female receiving a microbial sample. In one embodiment, the subject is a recipient of a composition comprising the SCVMP described herein, e.g., a recipient female. In one embodiment, the subject is a donor female providing a microbial sample. In some embodiments, a subject is a human female exhibiting a clinical condition related to a microbial imbalance (e.g., a dysbiosis).

The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “lower”, “reduced”, “reduction” or “decrease”, “down-regulate” or “inhibit” mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level (or levels below the limit of detection) as compared to a reference sample), or any decrease between 10-100% as compared to a reference level.

The term “substantially” means to a great or significant extent. For example in the case of a sample that is “substantially free of” pathogens, the term means the sample is, for the most part, or essentially, but possibly not completely, void of a pathogen. A sample that is substantially free of selected pathogens can refer to a sample that comprises about 5%, <5%, <4%, <3%, <2%, or <1% of the selected pathogens. In a particular embodiment, the term “substantially free” comprises <5%.

The term “strain” has no universally accepted definition, and as used herein refers generically to a sub-species categorization. In one embodiment, strains are represented by the bacterial haplotypes. In another embodiment, strains are analyzed and defined by whole genome sequencing. In one embodiment, strains share a sequence identity of at least 98%, 99%, 99.5%, or 99.9% in defined genetic region, such as an allele or microsatellite region. In a preferred embodiment, the defined genetic region is a conserved genetic region, wherein a “genetic region” refers to a stretch of nucleic acid, e.g. DNA, which is inheritable. In another embodiment, microbes belong to the same bacterial strain if the level of genomic diversity is lower within a strain compared to the level of genomic diversity among different strains. The genomes of the microbes belonging to the same strain thus form a genetic cluster of diversity that is less divers than when compared to another cluster of another strain.

The term “haplotype” refers to a set of DNA variations or polymorphisms, e.g. single nucleotide polymorphisms (SNPs), on the same chromosome or in defined genetic regions and that are inheritable. Hence, haplotypes can be used as indicators of strains.

“Substantially complete vaginal microbiota preparation(s)” is sometimes abbreviated as SCVMP herein.

### EXAMPLES

### Example 1

### Screening of Women to Identify Donors and Recipients

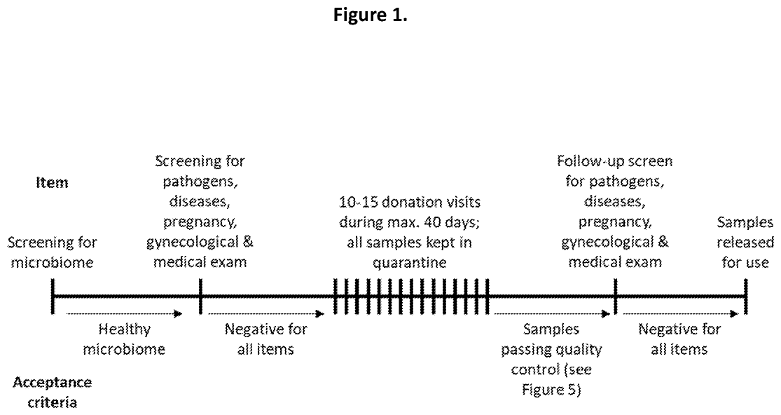

This Example describes the process of identifying suitable donors based on the analysis of the vaginal microbiota composition and absence of pathogens. A schematic overview of the screening is shown in FIG. 1.

### Participant Enrolment and Vaginal Samples

Healthy women without a history of vaginal health issues were recruited. Enrolling participants were informed, consented and provided a vaginal swab for microbiome analysis, and a separate swab for human papillomavirus (HPV) analysis. A total of 96 women consented and provided samples. For the last 60 women screened, instead of a vaginal swab, a cervicovaginal secretion (CVS) sample was obtained using a vaginal self-sampling device (e.g., a pliable menstrual cup that is inserted into the vaginal cavity, such as a Softdisc menstrual cup by The Flex Company, also sold under the name ‘Flex disc’) to enable both microbiome and biomarker analysis, as well as a dedicated swab for HPV screening. Both sample types were obtained via self-sampling by donors after thorough instruction of the donor on the correct sampling procedure.

### Microbiome Analysis

The microbiota composition from the vaginal swab or CVS samples was determined by shotgun DNA sequencing analysis. Swabs for vaginal microbiome analysis were kept at 4° C. for up to 48 hours prior to DNA extraction. CVS samples were diluted with saline to reduce the viscosity, aliquoted (for procedure, see Example 3) and stored at −80° C. prior to DNA extraction. DNA was extracted from both sample types using the Molysis Complete5 kit (MolZym), which uses a differential lysis method to extract microbial DNA and remove human DNA. Shotgun sequencing and bioinformatics analyses of the microbiomes was performed (Seqbiome, Cork, IE). All samples were prepared for shotgun metagenomic sequencing according to Illumina Nextera XT library preparation kit guidelines, with the use of unique dual indexes for multiplexing with the Nextera XT index kit (Illumina). Samples were sequenced on an Illumina NextSeq 500 sequencing platform with a v2.5 kit (a 300-cycle kit/150 bp PE sequencing) using standard Illumina sequencing protocols. Quality of the raw sequencing data was assessed using FastQC. Samples that did not meet minimum quality check cutoff or a sequencing depth of at least 4 million reads underwent re-sequencing. Shotgun metagenomic sequencing data were then processed through analysis workflow that utilizes Kneaddata wrapper tool. Quality filtering and host genome decontamination (human) were performed utilizing Trimmomatic and Bowtie2. Taxonomic classification of quality filtered reads was further performed using Kraken2 species classifier using a customized version of Genome Taxonomy Database (GTDB) that also includes reference sequences belonging to archaea, fungi and viral genomes. Kraken2 classifications were then passed to Bracken tool to estimate species level abundance. The vaginal microbiota composition was determined for each putative donor and recipient by obtaining information regarding the presence and relative abundance of bacterial species.

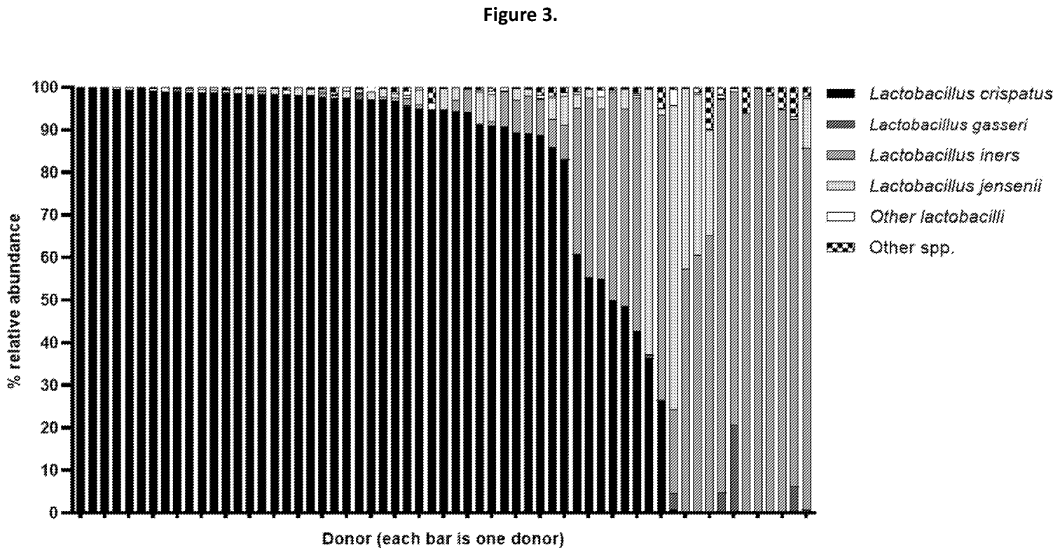

For this study, to qualify as a donor, the vaginal microbiota composition had to pass two criteria: to comprise at least 80% vaginal lactobacilli (L. crispatus, L. iners, L. jensenii, L. gasseri) and to comprise less than 5% selected pathogens (Atopobium spp., Prevotella spp., B. vaginale, and F. vaginae). The vaginal microbiota composition of 61 out of 96 women (64%) satisfied these two criteria and were considered healthy vaginal microbiomes. The relative abundance of the lactobacilli present in the vaginal microbiota are depicted in FIG. 3, wherein each bar represents the microbiota composition of a single healthy donor. The relative quantities of species are indicated on the y-axis. The microbiomes of the screened donors differed in their relative composition. In several instances, the microbiome was dominated by a single species, e.g., L. crispatus, wherein other microbiomes showed a heterogenous population of lactobacilli, e.g., comprising a combination of L. crispatus, L. iners and L. jensenii. Of note, even minute relative quantities, such as less than 0.05% could be detected (see, Table 2).

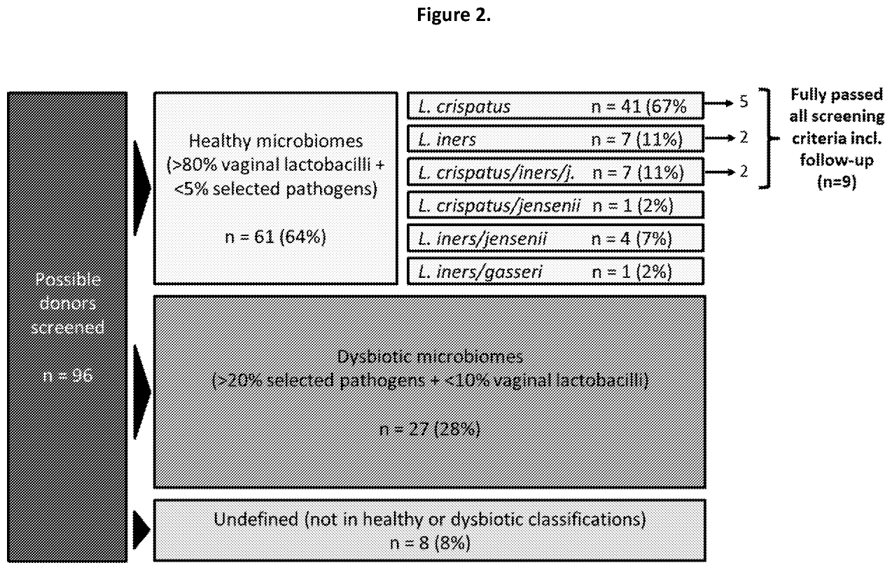

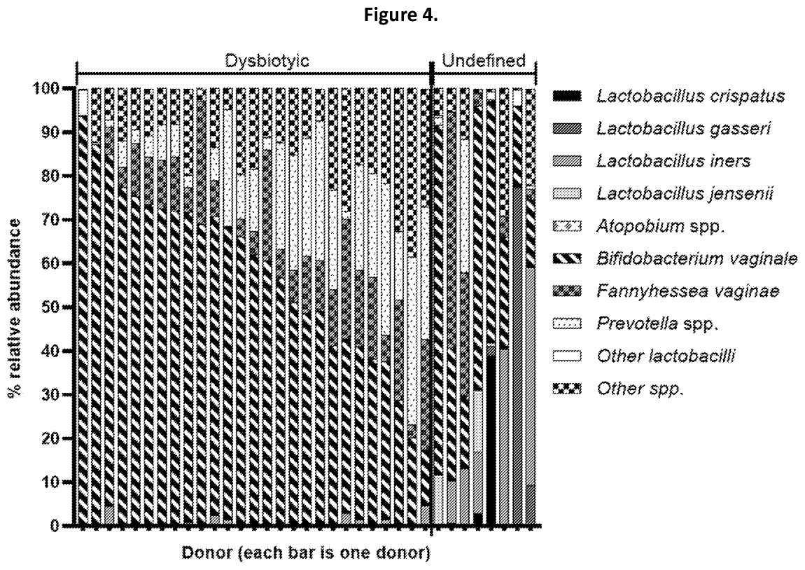

Of the 96 women screened, 28% (n=27) exhibited a dysbiotic microbiome (defined as >20% selected pathogens and <10% vaginal lactobacilli) (see, FIGS. 2 and 4). The women that exhibited dysbiotic vaginal microbiota were free of vaginal disease symptoms and of general good health. The remaining 8% (n=8) fell between the healthy and dysbiotic microbiome classifications, did not exhibit any vaginal disease symptoms, or contained species not classified/defined in the screening criteria (FIG. 4). These women did not qualify as putative donors or recipients.

The microbiota compositions of the healthy cohort (n=61) had a median vaginal lactobacilli abundance of 99.26% and selected pathogens of 0.03%. In contrast, the dysbiotic cohort (n=27) was characterized by a median vaginal lactobacilli abundance of 0.34% and selected pathogens of 87.21%. The undefined cohort (n=8) was characterized by a median vaginal Lactobacilli abundance of 35.76% and selected pathogens of 61.83%.

This suggests that the selection criteria (see, FIG. 2) can reliably distinguish healthy and dysbiotic vaginal microbiota with high certainty. Only healthy vaginal microbiota were considered as putative donors and underwent further stringent screening procedures.

### HPV Screening

Human papillomavirus (HPV) is a highly prevalent viral infection, several strains of which are associated with cervical cancer. The prevalence of HPV-positive women is high. On the vaginal swabs taken for HPV screening during the donor's screening visit, DNA extraction was performed using the Qiagen QIAmp DNA mini kit. Extracted DNA from each donor sample was used in the SeeGene Anyplex II HPV28 kit and ran semi-quantitatively on the BioRad CFX96 Dx qPCR machine, which was set up and calibrated for this assay (Triolab, DK). The semi-quantitative method provides three levels of quantity for 28 different genotypes of HPV, which included all known high-risk genotypes. Only samples that were HPV-negative were considered suitable putative donors.

### Lack of Pathogen Analyses and Examination by Medical Staff

To minimize any adverse effects for the recipients, putative donors (women who had a healthy microbiome and passed the HPV screen) were further screened by a gynecologist for the presence of pathogens, pathobionts, and sexually transmitted diseases, and underwent a medical and gynecological examination to assess the presence of other diseases such as cancer and endometriosis. The procedure included an additional HPV test.

Standard diagnostic tests were performed for HIV, Hepatitis A, B, and C, cytomegalovirus, Treponema, urinary tract infections, HPV, Chlamydia, gonorrhea, Trichomonas, Herpes genitalis, Candida, Mycoplasma, and Streptococcus A, B, C, and G. During the medical examination, putative donors were subjected to a general health check including medical history and medication usage, demographics, heart rate and blood pressure measurements.

For approved donors, the same tests were again performed at a follow-up visit after they had provided their last donations over a specified time period (see, FIG. 1). Until approved donors passed the test at the follow-up visit, and all samples passed all quality checks (see, Example 4), the samples remained quarantined and not released for administration to a recipient.

### Donor Selection

Donors were selected on the screening procedure described above, including microbiome analysis, pathogen screening, and medical and gynecological examination. Only subjects that had a healthy vaginal microbiome, had no positive pathogen test, and had no abnormal findings in the medical and gynecological exam were considered suitable donors and were enrolled in the program (see also, Example 2).

Following the screening protocol, 12 out of 96 screened women (13%) were enrolled into the donor program and started their donation visits. Of these, three failed to provide sufficient donations or failed during follow-up screening. Overall, of all 96 women who enrolled for screening 9% fully passed all screening criteria resulting in nine approved donors (n=9) (Table 1).

Table 1 provides an overview of the pathogens and pathobionts that were screened for in donor subjects (n=38).

TABLE 1

| | n positive |
|---|---|
| Screening | out of n = 38 |
| HIV | 0 |
| Hepatitis A/B/C | 0 |
| CMV IgM | 0 |
| Treponema | 0 |
| UTI | 2 |
| HPV | 13 |
| Chlamydia | 1 |
| Gonorrhoeae | 0 |
| Trichomonas | 0 |
| Herpes genitalis | 0 |
| Mycoplasma | 1 |
| Strep A/B/C/G | 4 |
| Candida | 7 |
| Other (e.g., abnormal gynecological findings) | 4 |
| Total passed - out of the original 96 women | 12 |
| Failed during donations or at follow-up | 3 |
| Total fully passed/approved donors | 9 |

The composition of the SCVMP of all screened subjects (n=96) including the fully approved donors (n=9) is summarized in Table 2.

Table 2: Vaginal microbiota compositions of four cohorts: (i) Healthy, (ii) fully approved donors, (iii) dysbiotic and (iv) undefined. Shown are the relative amounts of the quantified bacteria in median and interquartile range (IQR) (in %) relative to the total amount of detectable species in the sample preparation.

TABLE 2

| | Healthy | | Fully approved | | Dysbiotic | | Undefined | |
|---|---|---|---|---|---|---|---|---|
| | (n = 61) | | donors (n = 9) | | (n = 27) | | (n = 8) | |
| Species | median | IQR | median | IQR | median | IQR | median | IQR |
| Lactobacillus | 94.75 | 50.05 | 97.42 | 50.16 | 0.00 | 0.00 | 0.03 | 1.08 |
| crispatus | | | | | | | | |
| Lactobacillus gasseri | 0.00 | 0.08 | 0.00 | 0.00 | 0.00 | 0.00 | 0.28 | 3.84 |
| Lactobacillus iners | 0.84 | 42.20 | 0.84 | 46.48 | 0.34 | 1.11 | 11.47 | 20.79 |
| Lactobacillus jensenii | 0.74 | 2.82 | 0.07 | 1.34 | 0.00 | 0.00 | 0.10 | 3.42 |
| Total vaginal | 99.26 | 1.44 | 99.62 | 0.37 | 0.34 | 1.10 | 35.76 | 33.35 |
| lactobacilli | | | | | | | | |
| Atopobium spp. | 0.00 | 0.00 | 0.00 | 0.00 | 0.00 | 0.01 | 0.00 | 0.00 |
| Bifidobacterium | 0.01 | 0.30 | 0.00 | 0.08 | 61.60 | 31.71 | 27.98 | 39.65 |
| vaginale | | | | | | | | |
| Fannyhessea vaginae | 0.00 | 0.00 | 0.00 | 0.00 | 10.70 | 9.75 | 2.63 | 10.11 |
| Prevotella spp. | 0.00 | 0.01 | 0.00 | 0.00 | 10.23 | 22.19 | 0.02 | 0.46 |
| Total selected | 0.03 | 0.50 | 0.01 | 0.08 | 87.21 | 10.10 | 61.83 | 49.59 |
| pathogens | | | | | | | | |
| Other lactobacilli | 0.31 | 0.55 | 0.15 | 0.14 | 0.00 | 0.00 | 0.28 | 0.98 |
| Other species | 0.14 | 0.58 | 0.03 | 0.33 | 12.48 | 10.96 | 5.71 | 13.50 |

“Healthy”, “Dysbiotic” and “Undefined” cohorts were categorized based on the relative quantities of the vaginal lactobacilli and pathogens as described above (see also, FIG. 2). “Fully approved donors” refers to subjects that, in addition to passing the first screen successfully underwent additional screens, e.g., to exclude STIs and other infections and medical conditions as described above. Subjects that passed all screening criteria and qualified as suitable donors (n=9) had a median vaginal lactobacilli concentration of 99.62% and selected pathogens of 0.01%. Approved donors had the highest amount of selected vaginal lactobacilli and the lowest amount of vaginal pathogens of the groups. Fully approved donors were not limited to L. crispatus-dominant vaginal microbiota compositions but comprised a mixture of lactobacilli species or were L. iners dominant.

### Recipient Selection

Recipients were recruited and enrolled in clinical studies. In one study, healthy volunteer women aged 18 to 45 were recruited for screening and participation. Only women without vaginal disease symptoms and of general good health were enrolled for screening. Another study enrolled a single patient with vaginal symptoms. All recipients were classified as having vaginal dysbiosis (harboring a dysbiotic vaginal microbiome) according to the inclusion criteria defined herein (see, e.g., FIG. 2): >20% selected pathogens (Atopobium spp., Prevotella spp., B. vaginale, and F. vaginae) and <10% vaginal Lactobacilli.

### Example 2

### Obtaining Donor CVS Samples

This Example describes the process of obtaining vaginal microbiota samples from a donor, wherein the sample comprises substantially complete vaginal microbiota.

### Donation Visit Setup

Donors that successfully passed the screening process described in Example 1 were invited to donate cervicovaginal secretions (CVS) within a time period of 40 days between the two gynecologist/pathogen check visits (see, FIG. 1). Each donor provided 10-15 donations at any time during the 40 days except on days during menstruation and one day thereafter. The donations were spaced at least 16 hours apart.

During the donation period, donors needed to adhere to the following restrictions. abstinence from vaginal and anal sexual intercourse; no swimming in lakes, Jacuzzis and swimming pools; no use of intravaginal products (e.g., tampons, soap). At each visit, the donor filled out a questionnaire to affirm adherence to these restrictions together with general health questions.

After the donation period, the donors underwent a follow-up check at the gynecologist. Only if they passed this follow-up, were their samples considered for release and use for administration (see, FIG. 1, Example 1) Until that time, and until all samples passed all quality checks (see, Example 4), all samples were kept in quarantine.

### Donor Self-Collection of CVS

Cervicovaginal secretion (CVS) samples were obtained through self-collection using a vaginal self-sampling device after thorough instructions. Donors obtained CVS samples in a dedicated, hygienically designed donor room. The donor room was cleaned with 70% ethanol after each donor and subjected to biweekly environmental monitoring to check for Enterobacteriaceae, total aerobic microbial count, and yeast and mold. This setup maximizes cleanliness and minimizes processing time, compared to, e.g., home sampling. Only a single contamination was detected in one of the donor samples consisting of skin bacteria, caused by the donor not following the sampling procedure correctly.

The vaginal self-sampling device was a single use menstrual cup with a flexible/pliable ring and plastic foil cup (described in Example 1). For CVS donations, it was not worn like a menstrual cup over the cervix, but instead used as a large swab by inserting it partially folded into the vagina, leaving it in a longitudinal position for about 10 seconds and twisting it along its longitudinal axis while removing it. The donor deposited the vaginal self-sampling device into a provided labeled and pre-weighed sterile 50 mL tube. After 15-20 min, this process was repeated a second time with a new vaginal self-sampling device, which was placed in a separate sterile 50 mL tube.

The soft and pliable material of the vaginal self-sampling device in conjunction with it being inserted partially folded into the vaginal canal enables the effective collection of CVS from the surface of the vaginal cavity, without damaging or irritating the vagina. The vaginal self-sampling devices are intended and safe for vaginal use, but to ensure maximum donor safety and control, each batch of the devices was screened for contamination of Enterobacteriaceae and confirmed to be free of any contamination.

The procedure described here is believed to have several advantages over using the vaginal self-sampling device over a prolonged time period as a collecting device over the cervix. With the above method, the bacteria comprised in the CVS sample spend less time in contact with the vaginal self-sampling device, and the amount of vaginal mucus and vaginal bacteria that is collected is increased. The vaginal epithelium contains the substrates for the vaginal microbiota. Thus, a sample obtained in this manner is thought to have a higher concentration of viable vaginal lactobacilli and mucus, and a lower amount of fluid from the endometrium.

### Example 3

### Producing a SCVMP

This Example demonstrates the production of a SCVMP from cervicovaginal secretions.

### Process to Produce a SCVMP

A schematic representation of the sample processing is provided in FIGS. 5A and 5B. At each visit, the donor was provided with two 50 mL sterile tubes, which were pre-labelled and pre-weighed, and two vaginal self-sampling devices, along with the questionnaire (see, Examples 1 and 2). The CVS was collected by centrifuging for 5 min at 190×g and ambient temperature. The low speed collects the CVS from the device, while not phase-separating into layers. All further steps were conducted under sterile conditions at ambient temperature. The self-sampling devices were discarded from the tubes and the CVS sample weight was determined. Samples with a total (two tubes combined) weight of less than 200 mg were discarded. Samples in which blood was visibly present were discarded.

The sample in each tube was mixed with 1 mL saline to reduce viscosity, after which the two samples were combined and aliquoted for storage and further testing. The two combined samples were then distributed over several aliquots for quality control (see, Example 4) and storage as shown in FIG. 5B.

The cryovials containing samples for storage were placed in a CoolCell (Corning) and then at −80° C. A CoolCell has a controlled cooling rate of 1° C. per minute, which ensures maintaining maximum viability of the samples. After a minimum of 2 hours, the samples were transferred into a regular −80° C. storage box labelled ‘quarantined’. Samples were released only after all release criteria had been met (see, FIG. 5B and as described above).

### Example 4

### Quality Control

This Example describes the quality control of a SCVMP produced from cervicovaginal secretions obtained from the donor female, so it can be used safely for administration.

### Overall Procedure

A schematic representation of the sample processing procedure with details about quality control is provided in FIGS. 5A and 5B This procedure is highly optimized, in such a way that the volumes used for analyses and quality control are as low as possible to minimize loss of SCVMP. The sample used for pH measurement is discarded after measurement. The retention vial is maintained for at least 1 year after administration for safety reasons (e.g., to check for STIs in case one occurred in a recipient). Every first and last sample of each donor was subjected to a qPCR analysis to check for the absence of multi-drug resistance genes (MDR genes).

### Analyses for Quality Control

The pH was measured using a micro-pH probe. Technical triplicate measurements were performed, and repeated if the difference between measurements was >0.2. The sample used for pH measurement is discarded after measuring. Microscopy was performed. The absence of sperm cells was determined by microscopy and using acid phosphate paper, a highly sensitive and selective method to identify sperm cells. Viability was tested by colony-forming units (CFU) count on de Man, Rogosa and Sharpe (MRS) agar plates or with viable cell count (VCC) for donors who at screening were dominant in L. crispatus, L. jensenii, or L. gasseri. For donors who at screening were dominant for L. iners, this method could not be used. Instead, viability for these samples was tested using BacLight live/dead staining (ThermoFisher Scientific) and counting the viable cells/mL in a Thoma cell counting chamber under the microscope. Within one week after a sample was used for administration, a stability vial was used to test cell viability at the time of vaginal administration, and dose calculation.

DNA extraction was performed using the Molysis Complete5 kit (Molzym) according to the manufacturer's instructions to obtain sufficient bacterial sequence reads to perform in silico engraftment check after administration based on metagenome data (see, Example 1 for methods used). The multi-drug resistance (MDR) marker qPCR was performed using the same DNA as was used for Shotgun sequencing, using the SeeGene Allplex Entero-DR qPCR assay kit on a BioRad CFX96 Dx qPCR machine calibrated for the assay. This kit allows single or multiple detection of carbapenemase genes (NDM, KPC, OXA-48, VIM, IMP), extended spectrum beta-lactamase (ESBL) genes (CTX-M), and vancomycin resistance genes (VanA, VanB).

### Example 5

### Donor Microbiome Characterization (Species Level)

This Example describes the SCVMPs suitable for vaginal administration.

### Microbiota Compositions During the Donation Period

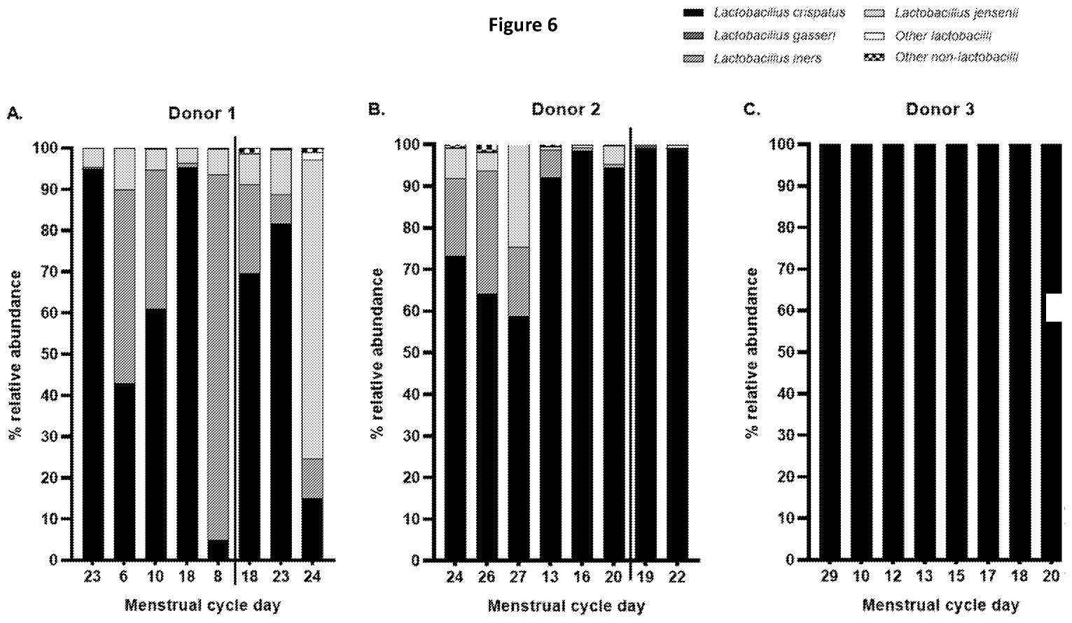

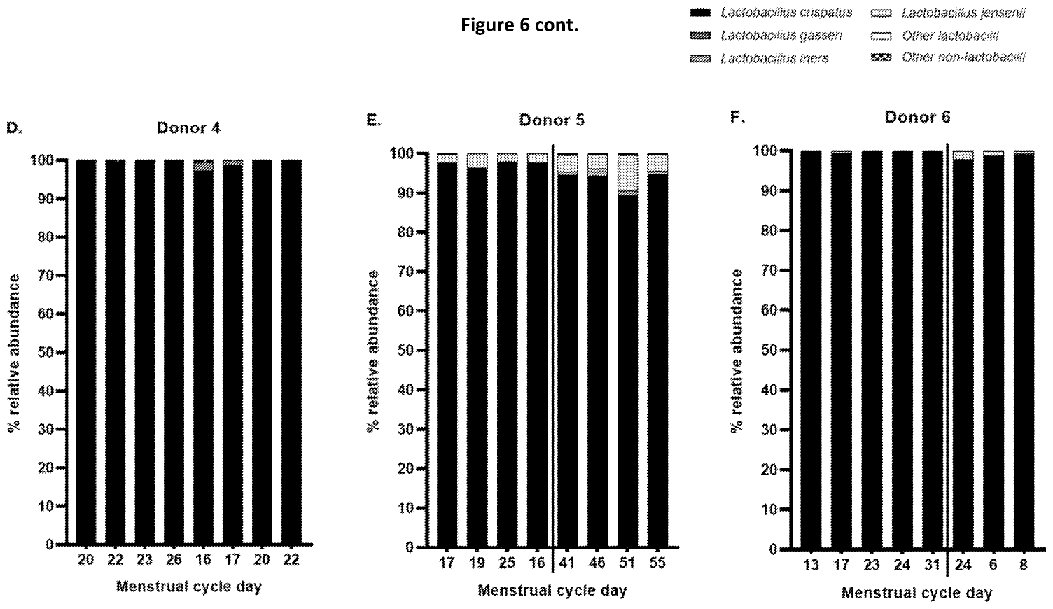

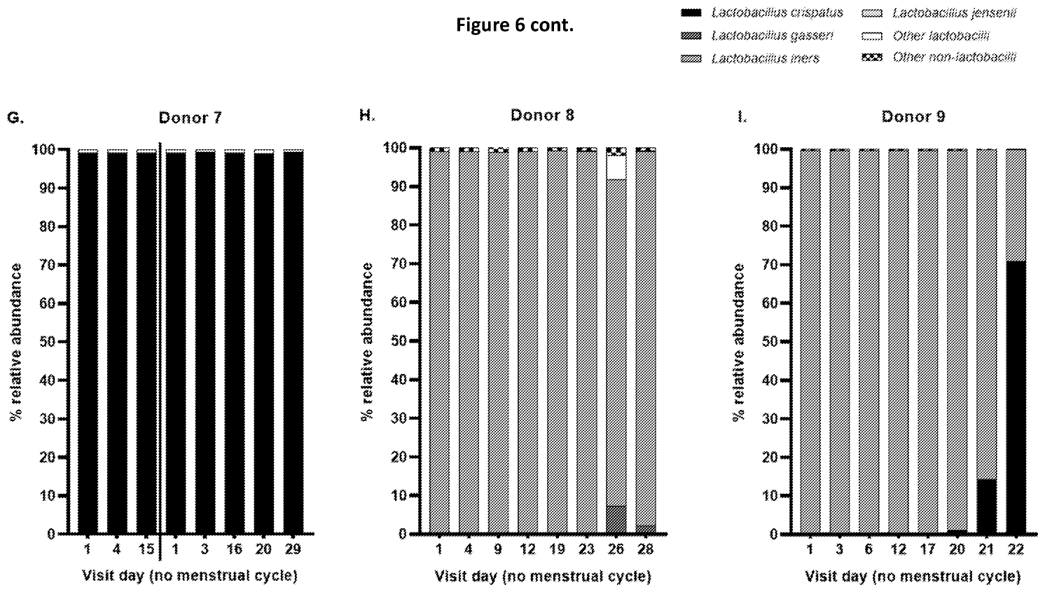

It is known that the vaginal microbiome in women can fluctuate over the menstrual cycle or depending on sexual activity (Gajer et al., 2012, Science Translational Medicine). For donors with mixed species compositions that did not use contraception (Table 3), fluctuations between the species were observed over the menstrual cycle (FIGS. 6A, 6B), but the totality of L. crispatus, L. iners, L. jensenii, L. gasseri in the sample remained stably >80%. The microbiome of donors with a strongly L. crispatus-dominant microbiome was very stable over extended periods of time (e.g., Donor 7, FIG. 6G).

The microbiome of L. iners-dominant donors was mostly stable, although in one case a shift between L. iners and L. crispatus was observed towards the end of the donation period (FIG. 6I). This donor did not have a menstrual cycle due to contraception. The microbiome stability observed in the donors is likely aided by the restrictions that the donors adhere to, such as, e.g., no sexual intercourse.

Some donors returned for a second round of donation visits. In FIG. 6, this is indicated by a vertical line. There was a 1 month pause in-between two rounds of donations for donors 2, 5, and 7, and a 1,5-months pause for donor 1. For donor 6, there was a 2-months pause in-between donation rounds, and she changed from using contraceptive pills in the first round to no hormonal contraceptives in the second round.

In several donor samples, a certain species (mostly L. jensenii) was present in small but very consistent amounts (0.3-3.5%). In one of these donors, an even smaller amount of L. iners was consistently present (0.07-0.33%). Small amounts of both L. iners (0.02-0.10%) and L. jensenii (0.01-0.03%) were consistently observed in yet another L. crispats-dominant donor. In the two donors with mixed species composition, none of the species present in the mixture fully disappeared (below detection level) over the donation period even though their relative abundances sometimes decreased to as low as 0.27%. Altogether, this implies that the healthy vaginal microbiome is a stable ccosystem and points to a symbiotic relationship between the different Lactobacillus species in one person. This is further substantiated by the observations of the vaginal administration of the preparations, as described in Example 6.

In addition to the four main vaginal lactobacilli (L. crispatus, L. iners, L. jensenii, L. gasseri), other lactobacilli were regularly observed to be consistently present in small amounts in donor samples (mostly <0.05%): L. acetotolerans, L. acidophilus, L. amylovorus, L. gallinarum, L. gigeriorum, L. helveticus. L. johnsonii, L. kefiranofaciens, L. kitasatonis, L. paragasseri, L. psittaci, Lactobacillus sp002911475, L. taiwanensis, L. ultunensis, L. coleohominis, L. reuteri, and L. vaginalis. The stable presence of these lactobacilli further points to a complex ecosystem that in its entirety might be needed to promote and maintain a healthy vaginal niche.

Some other lactobacilli appeared sporadically in a subset of the donor samples. L. helveticus was not observed in the two L. iners-dominant donors, and only appeared in the last visit of a single donor who shifted from L. iners-dominant to L. crispatus-dominant, which may suggest that certain species prefer cohabitation with each other while others might be incompatible in the ecosystem. In contrast to reports in the literature where L. iners-dominant donors were not considered suitable for administration, the data suggest that L. iners-dominant microbiomes of fully approved donors may have a very stable and healthy ecosystem and are suitable donors for SCVMPs.

L. gasseri was infrequently observed as a dominant species (FIG. 3) but was typically found in small amounts in several of the donors (FIG. 6, Table 1). L. gasseri was detected in L. crispatus-dominant donor 4 (FIG. 6D) in relative concentrations of 0.01-2.23%; in mixed microbiome donor 1 (FIG. 6A) in relative concentrations of 0.01-0.07%; and in L. iners-dominant donor 8 (FIG. 6H) in relative concentrations of 0.3-7.14%. The consistent maintenance of minor Lactobacillus species suggests that the minor species might play a hitherto unknown role in maintenance or stability of the vaginal ecosystem.

### Physical Properties of the Obtained Samples

The weight of samples fluctuated among donations of the same donor and among donors, but on average was >200 mg. CFU and pH remained stable and within the defined threshold of <4.5 (Table 3). Altogether, this stability over both the microbiota composition and sample properties indicates the health and stability of the donor microbiomes.

Table 3 summarizes the properties of the CVS and SCVMPs that were generated. Each row contains data for one donor during her 10-15 donations over a 40-day period. The ‘Time window’ column lists the number of days during which the donor provided the number of samples listed in the ‘Nr of donations’ column. Donor ID is the same as in the microbiome graphs in FIG. 6. The weight is the combined weight of the two samples provided by the donor, prior to adding saline. The dose is the final amount of mL in the substantially complete vaginal microbiota preparation vial used for dosing a recipient, after all volumes for analyses have been taken out (see, FIG. 5 B). The maximum dose is 1.8 mL to fit in a cryovial. Viable cells/dose was determined by CFU plate counting on MRS medium grown anaerobically for 2-3 days for all species except for the two L. iners-dominant donors (8 and 9). For these, viable cells/dose was determined by live/dead staining and counting viable cells in a counting chamber under the microscope. Abbreviations: hIUD: hormonal intrauterine device; pill: oral contraceptive

TABLE 3

| | | | | Time | Ranges (min-max) | | | | Averages | | | |
|---|---|---|---|---|---|---|---|---|---|---|---|---|
| Donor | | | No of | window | Weight | Dose | | Viable | Weight | Dose | | Viable |
| ID | Microbiome | Contraception | donations | (days) | (mg) | (mL) | pH | cells/dose | (mg) | (mL) | pH | cells/dose |
| 1 | Mixed | none | 15 | 28 | 244-1035 | 1.5-1.8 | 3.9-4.4 | 8.6E+06- | 515 | 1.6 | 4.1 | 1.2E+08 |
| | | | | | | | | 3.1E+08 | | | | |
| 2 | Mixed | none | 10 | 36 | 311-918 | 1.4-1.8 | 4.1-4.3 | 1.9E+08- | 597 | 1.7 | 4.2 | 3.6E+08 |
| | | | | | | | | 5.4E+08 | | | | |
| 3 | L. crispatus | none | 11 | 25 | 628-1778 | 1.7-1.8 | 3.7-3.8 | 5.1E+07- | 1206 | 1.8 | 3.8 | 1.5E+10 |
| | | | | | | | | 8.4E+10 | | | | |
| 4 | L. crispatus (+some | hIUD | 14 | 31 | 195-486 | 1.3-1.7 | 3.9-4.2 | 7.2E+07- | 334 | 1.5 | 4.1 | 1.9E+08 |
| | L. gasseri) | | | | | | | 3.4E+08 | | | | |
| 5 | L. crispatus + | pill | 12 | 30 | 340-823 | 1.5-1.8 | 3.7-4.0 | 3.3E+08- | 525 | 1.7 | 3.9 | 7.4E+09 |
| | L. jensenii | | | | | | | 1.0E+11 | | | | |
| 6 | L. crispatus + | pill | 14 | 20 | 276-703 | 1.3-1.8 | 3.8-3.9 | 5.0E+07- | 487 | 1.6 | 3.8 | 1.7E+10 |
| | L. jensenii | | | | | | | 1.0E+11 | | | | |
| 7 | L. crispatus + other | pill | 13 | 16 | 376-710 | 1.6-1.8 | 3.8-4.0 | 3.6E+08- | 542 | 1.7 | 3.9 | 8.5E+09 |
| | lactobacilli | | | | | | | 1.0E+11 | | | | |
| 8 | L. iners (+L. | hIUD | 12 | 31 | 532-1593 | 1.6-1.8 | 3.9-4.0 | 4.3E+08- | 868 | 1.8 | 3.9 | 8.5E+08 |
| | gasseri towards | | | | | | | 1.2E+09 | | | | |
| | end) | | | | | | | | | | | |
| 9 | L. iners (+ L. | hIUD | 14 | 24 | 233-627 | 1.4-1.8 | 3.9-4.2 | 1.3E+08- | 446 | 1.6 | 4.0 | 3.0E+08 |
| | crispatus towards | | | | | | | 6.0E+08 | | | | |
| | end) | | | | | | | | | | | |
| Average | | | 13 | 27 | | | | | 614 | 1.6 | 4.0 | 5.5E+09 |

It was further evaluated whether repeated CVS donations would affect the composition of the sample. Donors provided samples 2 times per day with a 6-hour interval and 3 times per day with a 4-hour interval in between samples. Both of the repeated CVS donation procedures resulted in a significant increase of the pH (approx. 0.4 increase), and a decrease of sample weight (to around or below 200 mg) and CFU (1 log), rendering repeated sampling within a single day unsuitable. This suggests that the microbiota require more than 6 hours to fully regenerate after sampling. Consequently, donors were allowed to give samples once a day at most with approximately 16-hour intervals between donations.

### Stability of the SCVMP

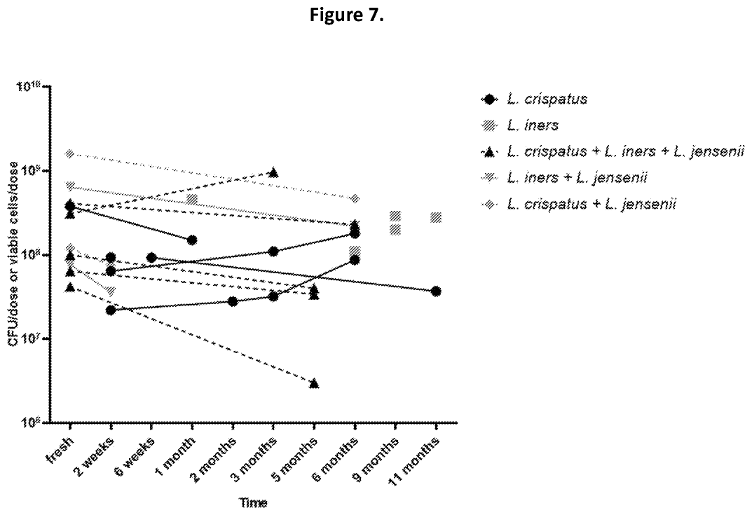

The stability of SCVMPs comprising different vaginal microbiota compositions was tested after different storage periods between 2 weeks to 11 months (FIG. 7). Stability was tested on 50 μL aliquots, which was found to be representative for the full volume vials (data not shown).

Generally, viability remained high over time for all species with no significant loss observed (average loss of viability below 1-log). No major differences in viability between species were observed. Other researchers have reported 2-log viability decreases after 9 months for vaginal microbiota compositions that are not L. crispatus-dominant. The improved stability seen here might, at least in part, be due to the collection method of obtaining the cervicovaginal secretion provided herein, which does not rely on a prolonged time period the collection cup is worn inside the donor subject but instead exposes vaginal mucus and bacteria only briefly to the collection cup. Without being bound by theory, it is believed that the longer the lactobacilli reside in the menstrual cup without contact or being able to adhere to the vaginal epithelium, the worse the viability, which might be particularly relevant for sensitive species, such as L. iners.

The process of obtaining the samples and processing the same described herein provides a SCVMP that is stable over extended periods of time for all tested species compositions.

Further, it was assessed whether the mode of thawing the samples affected viability of the lactobacilli comprised in the preparation. When performing vaginal administration of the preparation in the clinic, the clinician will generally pre-warm all materials needed to 30-37° C. Sample viability was tested after thawing the sample and warming it up to approx. 30° C. by two alternative methods: (i) thawing from −80° C. to 4° C., then to room temperature (RT) and 30° C., or (ii) the same procedure but without the 4° C. step.

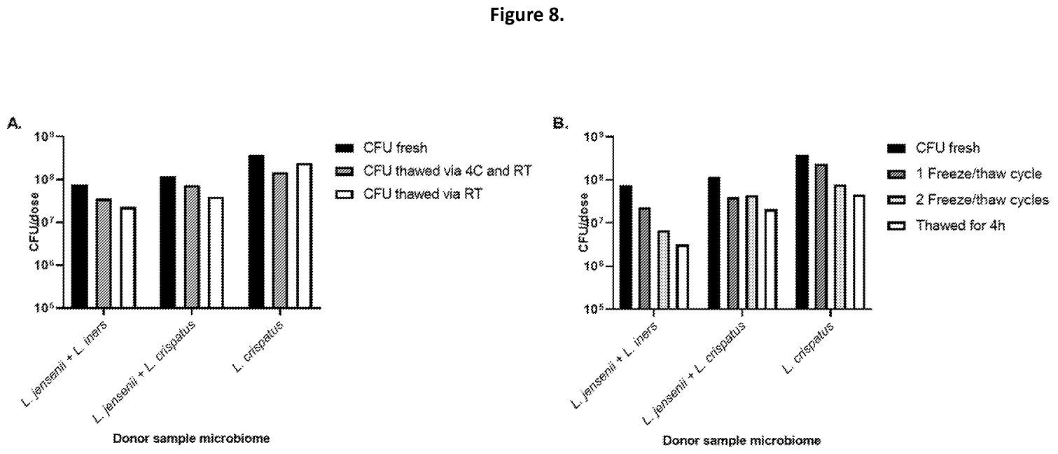

FIG. 8A shows the results, wherein ‘CFU fresh’ is the CFU obtained when plating the fresh sample directly after sampling and prior to any storage. ‘CFU thawed via 4C and RT’ is the CFU after taking the sample out of −80° C. storage and gradually warming it up by placing it at 4° C. for 30 min, then at room temperature (RT) for 30 min, and then at 30° C. ‘CFU thawed via RT’ skips the step at 4° C.

CFU and cell viability remained stable in all tested samples, wherein the former method resulted in slightly better viability for 2 samples, while for a third one the latter method was slightly better (FIG. 8A). However, differences were small and generally acceptable viability was maintained. This suggests that the 4° C. step can be omitted, and method (ii) was used for implementation in the clinic during vaginal administration of the preparations.

### Repeated Freeze-Thaw Cycles

Another aspect of stability is the capacity to withstand repeated freeze-thaw cycles. This is practically relevant in cases where recipients fail to report for an appointment to receive the SCVMP, and the samples might have to be re-frozen for administration at a later time point. Alternatively, shipments might be delayed and need refreezing after thawing while in transport. Hence, the effect of freeze-thaw cycles on the samples, as well as a prolonged incubation at room temperature (RT) were assessed.

FIG. 8B shows preliminary data, wherein ‘CFU fresh’ is the CFU as obtained when plating the fresh sample directly after sampling and prior to any storage. ‘1 Freeze/thaw cycle’ is the CFU after taking the sample out of −80° C. storage and leaving it at RT for about 40 min and pre-warming it to around 30-37° C. during about 30 min. ‘2 Freeze/thaw cycles’ is the CFU after repeating this freeze-thaw cycle one more time but without heating to 30-37° C., with the sample being stored at −80° C. in-between thaw cycles for 2-3 weeks. ‘Thawed for 4 h’ is the CFU after taking the sample out of −80° C. storage and leaving it at room temperature (RT) for 4 hours instead of 30 min prior to plating. The results indicate that undergoing the freeze-thaw cycle twice reduces viability more than undergoing the cycle just once. L. crispatus-L. jensenii mixtures were most robust and could withstand repeated freeze-thaw cycles better than other tested compositions (FIG. 8B).

A prolonged (4 h) incubation at RT reduced viability by more than 1 log or close to 1 log and hence should be avoided. These preliminary data demonstrate that all samples could maintain cell viability for at least one freeze-thaw cycle, when not left for longer periods at RT. This is useful in a clinical setting, where samples can be refrozen and do not need to be discarded.

### Example 6

### Changing Dysbiotic Microbiota Through Vaginal Administration of the Preparation and Stable Engraftment

This Example demonstrates how a SCVMP can be used to revert a recipient's dysbiotic microbiome to a donor's healthy microbiome using vaginal administration, and how the information can be used to select engrafting strains.

### Administration of SCVMP and Results

The SCVMP was first thawed by moving it from −80° C. to room temperature for 40 minutes. Thereafter, the sample was placed in an incubator at 37° C. for 30 minutes. The tube containing the preparation was then gently inverted S times for mixing. The preparation was drawn up into a 5 mL syringe and applied intravaginally to the recipient lying in the lithotomy position using an insemination catheter attached to the 5 ml syringe. The recipient was instructed to remain lying down in a horizontal position for 30 minutes following the sample being inserted intravaginally.

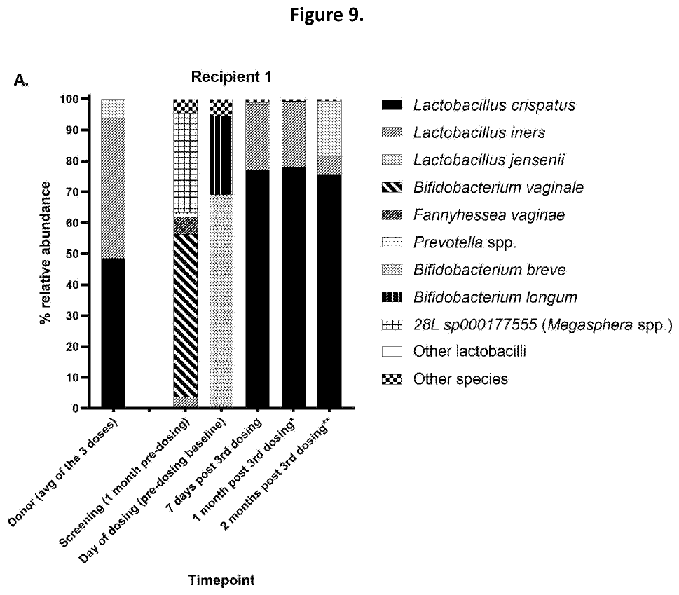

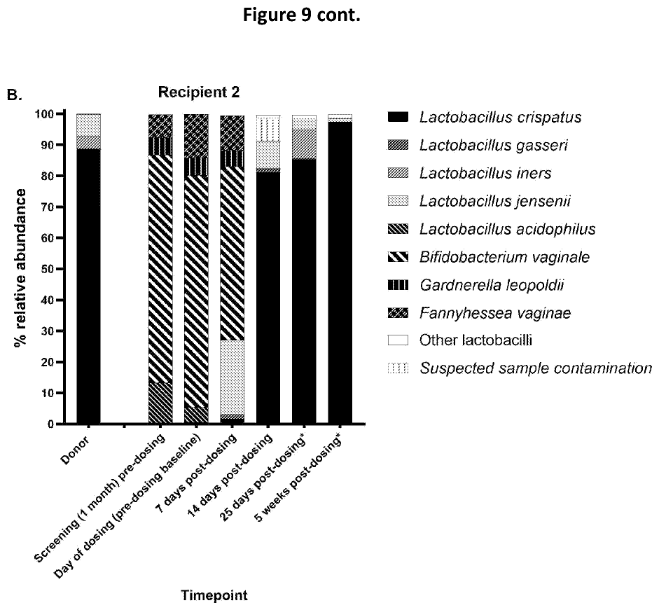

Two representative changes of a dysbiotic vaginal microbiota after administration of a SCVMP are shown in FIG. 9. Recipient 1 received 3 doses (1 dose/day over the course of 3 consecutive days, in the range of 4.2×10⁷-10×10⁸ CFU/dose) (FIG. 9A), while recipient 2 received only a single dose of 3.1×10⁸ CFU/dose (FIG. 9B). To increase the chance of success for recipient 2, an in vitro donor-recipient matching assay was performed as described in Example 6. The sample that performed best in that assay, from donor 1 (see, Table 4), was successfully used for administration in recipient 2. The recipients were not pre-treated with antibiotics prior to or during the administration of the preparation. After administration of the preparation, the microbiome changes of the recipients demonstrate that the SCVMP is capable of changing a dysbiotic microbiome to a microbiome more closely resembling that of the donor, by administration of the SCVMP alone (without treatment with antibiotics).

All recipients were enrolled based on their microbiome at screening, which was generally one month before their visit. At their visit for administration of the SCVMP, their microbiome was analyzed again as baseline measurement. Recipient 1 was vaginally asymptomatic, while recipient 2 showed vaginal disease symptoms at the time of administration. Recipient 2 reported a marked decrease in symptoms one week after administration of the preparation, and full disappearance of all vaginal disease symptoms after two weeks.

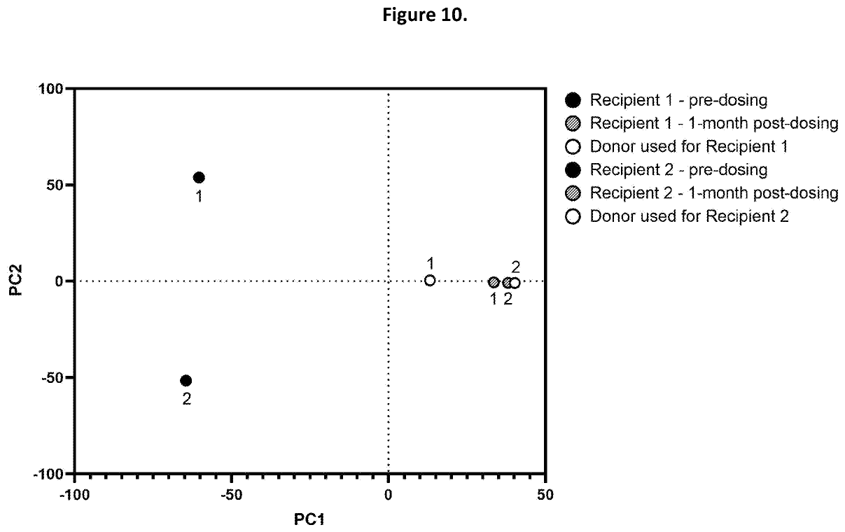

The post-administration microbiome of the recipients appeared similar to the donors' and remained fairly stable. Overall, the post-administration recipients clustered together in an unsupervised principal component analysis (PCA) plot with the donor samples, while the pre-administration recipients form a separate cluster (FIG. 10). This suggests that the vaginal microbiota of the recipient after administration of the preparation did not only change from a dysbiotic state but resembles the vaginal microbiota composition of the healthy, non-dysbiotic donor.

The microbiota compositions of the recipients pre- and post-administration of the SCVMP(s), and the composition of the respective donor are summarized in Table 4. The Table shows the relative abundances (determined by Shotgun sequencing) of species in donor and recipient samples. Donor species engrafted in the vaginal niche of recipients, including species that were present in the donor in low quantities but absent in the recipient prior to administration of the preparation.

The relevant species for which this phenomenon is observed are highlighted in the grey columns. The post-administration microbiome of the recipient included species that were present in minor quantities in the donor microbiome (Table 4; wherein L. is Lactobacillus and B. is Bifidobacterium), suggesting that the entire stable ecosystem was transferred from the donor to the recipient. It was also observed that these species were stably present for all donation visits of most donors (see, Example 5). SCVMPs were effective in both a single and a multiple dose in changing the recipient's vaginal microbiota composition.

Other researchers have previously suggested a harmful role of L. iners. Without being bound by theory, it is believed that beneficial and less beneficial strains of L. iners exist. L. iners is present in many of the donor microbiomes that were tested. In women that pass the stringent screening criteria described herein, L. iners may play a beneficial role in a healthy, stable microbiome.

This Example thus demonstrates the successful change of a dysbiotic vaginal microbiome by administration of a SCVMP obtained from a healthy donor female to a dysbiotic recipient, wherein post-administration the recipient's microbiota composition closely resembled the donor's microbiota composition.

The successful engraftment of the donor's SCVMP allows the subsequent identification of bacterial species and strains as discussed in detail in the following examples. In particular, not only the identification of major strains, but also minor strains, e.g., strains that are present in low relative abundance in the SCVMP, can be identified. The identification of a single bacterial strain or a plurality of bacterial strains (e.g., a bacterial consortium) that are capable of engrafting in the recipient's vaginal niche and improving the recipient's dysbiosis cannot be identified by in vitro methods using classical “reverse” microbiological screens. Known methods in the art aim to identify lactobacilli that may potentially engraft in the vaginal niche based on known functions thought of as being predictive (based on work with known lactobacilli) and advantageous in vitro growth. There is a question, however, how predictive for successful engraftment these functions are. The present method first determines the desired outcome (e.g., change of dysbiosis) and subsequently identifies the lactobacilli are capable of engrafting that brought about the change. This “forward” screen approach allows not only the identification of lactobacilli that are capable of engrafting and improving dysbiosis in the recipient's vaginal tract but does so with a higher degree of success than with in vitro methods. The present method has a higher chance of identifying effective strains that are capable of improving dysbiosis and engraftment. In addition, the method allows the identification of strain consortia (a plurality of strains that are well adapted to thrive with each other), e.g., by combining two, three, four or more strains that were isolated from the same donor sample.

A further advantage lies in the capability of identifying strains that are present in very low relative quantities, e.g., at levels below 0.5%, 0.1% or less than 0.01%. The Lactobacillus strain(s) that are identified to be capable of engrafting in the recipient's vaginal niche may be combined into a consortium of lactobacilli which are capable of engrafting in a dysbiotic vaginal niche. The present method thus allows the identification of lactobacilli strains or consortia thereof that are capable of engrafting irrespectively of having a previously known function in the vaginal niche. Details of the various steps of the method are exemplified below.

TABLE 4
Relative abundance (%) of vaginal Lactobacillus species and selected vaginal pathogens
after administration of the substantially complete vaginal microbiota preparation.

| Recipient | L. | L. | L. | L. | L. | Limosilactobacillus | Prevotella | B. |
|---|---|---|---|---|---|---|---|---|
| 1 | crispatus | gasseri | iners | jensenii | helveticus | vaginalis | spp. | vaginale |
| Dose 1 | 42.84 | 0.00 | 47.17 | 9.84 | 0.01 | 0.02 | 0.00 | 0.00 |
| donor | | | | | | | | |
| sample | | | | | | | | |
| Dose 2 | 46.65 | 0.00 | 48.30 | 4.89 | 0.00 | 0.05 | 0.00 | 0.00 |
| donor | | | | | | | | |
| sample | | | | | | | | |
| Dose 3 | 56.07 | 0.00 | 39.78 | 3.83 | 0.00 | 0.10 | 0.00 | 0.00 |
| donor | | | | | | | | |
| sample | | | | | | | | |
| Screening | 0.02 | 0.00 | 3.63 | 0.00 | 0.00 | 0.00 | 1.29 | 52.64 |
| Day of | 0.08 | 0.00 | 0.02 | 0.00 | 0.00 | 0.00 | 0.55 | 0.04 |
| (pre-) dosing | | | | | | | | |
| 7 days post | 77.05 | 0.00 | 21.17 | 0.50 | 0.02 | 0.02 | 0.31 | 0.00 |
| 3rd dosing | | | | | | | | |
| 1 cycle post | 77.85 | 0.00 | 21.28 | 0.08 | 0.02 | 0.12 | 0.01 | 0.03 |
| 3rd dosing | | | | | | | | |

| | | | | 28L sp000177555 | | |
|---|---|---|---|---|---|---|
| Recipient | Fannyhessea | B. | B. | (Megasphera | Other | Other |
| 1 | vaginae | breve | longum | spp.) | lactobacilli | species |
| Dose 1 | 0.00 | 0.00 | 0.00 | 0.00 | 0.11 | 0.04 |
| donor | | | | | | |
| sample | | | | | | |
| Dose 2 | 0.00 | 0.00 | 0.00 | 0.00 | 0.09 | 0.04 |
| donor | | | | | | |
| sample | | | | | | |
| Dose 3 | 0.00 | 0.00 | 0.00 | 0.00 | 0.10 | 0.05 |
| donor | | | | | | |
| sample | | | | | | |
| Screening | 5.75 | 0.00 | 0.00 | 32.23 | 0.00 | 4.44 |
| Day of | 0.00 | 68.49 | 25.31 | 0.00 | 0.00 | 5.53 |
| (pre-) dosing | | | | | | |
| 7 days post | 0.00 | 0.00 | 0.00 | 0.00 | 0.04 | 0.92 |
| 3rd dosing | | | | | | |
| 1 cycle post | 0.00 | 0.00 | 0.00 | 0.00 | 0.16 | 0.57 |
| 3rd dosing | | | | | | |

| Recipient | L. | L. | L. | L. | L. | Limosilactobacillus | L. |
|---|---|---|---|---|---|---|---|
| 2 | crispatus | gasseri | iners | jensenii | helveticus | vaginalis | acidophilus |
| Donor | 88.71 | 0.05 | 4.03 | 7.16 | 0.02 | 0.01 | 0.00 |
| sample | | | | | | | |
| (1 dose) | | | | | | | |
| 26 days | 0.03 | 0.00 | 0.03 | 0.00 | 0.00 | 0.00 | 13.30 |
| pre- | | | | | | | |
| dosing | | | | | | | |
| Day of | 0.36 | 0.00 | 0.24 | 0.00 | 0.00 | 0.00 | 4.85 |
| (pre-) | | | | | | | |
| dosing | | | | | | | |
| 7 days | 1.70 | 1.14 | 0.45 | 23.80 | 0.08 | 0.03 | 0.00 |
| post- | | | | | | | |
| dosing | | | | | | | |
| 14 days | 81.20 | 1.10 | 0.13 | 8.90 | 0.80 | 0.00 | 0.02 |
| post- | | | | | | | |
| dosing | | | | | | | |
| 25 days | 85.30 | 0.24 | 9.40 | 3.76 | 0.77 | 0.07 | 0.02 |
| post- | | | | | | | |
| dosing * | | | | | | | |
| 5 weeks | 97.30 | 0.05 | 0.03 | 1.10 | 0.94 | 0.00 | 0.02 |
| post- | | | | | | | |
| dosing * | | | | | | | |

| | | | | | Suspected |
|---|---|---|---|---|---|
| Recipient | B. | Fannyhessea | Gardnerella | Other | sample |
| 2 | vaginale | vaginae | leopoldii | lactobacilli | contamination |
| Donor | 0.00 | 0.00 | 0.00 | 0.02 | 0.00 |
| sample | | | | | |
| (1 dose) | | | | | |
| | | | | 0.00 | |
| 26 days | 73.50 | 7.50 | 5.50 | 0.00 | |
| pre- | | | | | |
| dosing | | | | | |
| Day of | 74.80 | 14.10 | 5.60 | 0.00 | |
| (pre-) | | | | | |
| dosing | | | | | |
| 7 days | 56.00 | 11.20 | 5.10 | 0.00 | |
| post- | | | | | |
| dosing | | | | | |
| 14 days | 0.01 | 0.00 | 0.00 | 0.17 | 7.24 |
| post- | | | | | |
| dosing | | | | | |
| 25 days | 0.03 | 0.00 | 0.00 | 0.09 | |
| post- | | | | | |
| dosing * | | | | | |
| 5 weeks | 0.01 | 0.00 | 0.00 | 1.23 | 0.12 |
| post- | | | | | |
| dosing * | | | | | |

### Example 7

### Donor Microbiome Characterization (Haplotype Level)

This example describes the characterization of the donor microbiome at the strain level, wherein a strain can be identified by a distinct bacterial haplotype. The haplotypes act as an indicator or measure of the strains present in the SCVMPs suitable for vaginal administration.

While Example 5 described the donor samples on a species-level, this Example describes them on a haplotype or strain level. Haplotypes are a computational grouping of genomic variants (or polymorphisms that are inherited together). Haplotypes can be seen as indication of strains, where the haplotypes are defined by technical parameters such as selected genes and the number of genomes used as input. Haplotypes are typically used to assess the variety of sequences present on a sub-species level, allowing a detailed and deeper-level comparison of different metagenomes. Different methods can be used to identify haplotypes, and each will result in slightly different numbers of haplotypes (e.g., Chen C et al. Reconstruction of Microbial Haplotypes by Integration of Statistical and Physical Linkage in Scaffolding, Molecular Biology and Evolution, Vol 38, 2021, p. 2660-2672) but all will provide distinguishable haplotype signatures, enabling the sub-species level comparison of donors and recipients and hence confirmation of engraftment.

Here, StrainFinder (Smillie C S et al. Strain Tracking Reveals the Determinants of Bacterial Engraftment in the Human Gut Following Fecal Microbiota Transplantation. Cell Host Microbe. 2018; 23 (2): 229-240;) was used to determine the number of haplotypes. The haplotype analysis was performed on single copy core genes (n=1529 in this case), which were identified from metagenome assembled genomes (MAGs) reconstructed from the donor samples and a full-length isolate genome sequenced and assembled from one of the donor samples. Metagenomic reads were mapped onto a reference genome (an isolate genome of the target species; selected based on completeness/redundancy threshold) and single nucleotide polymorphisms (SNPs) were identified in the single copy core gene regions, which were subsequently used as an input to an algorithm that predicts the number of haplotypes.

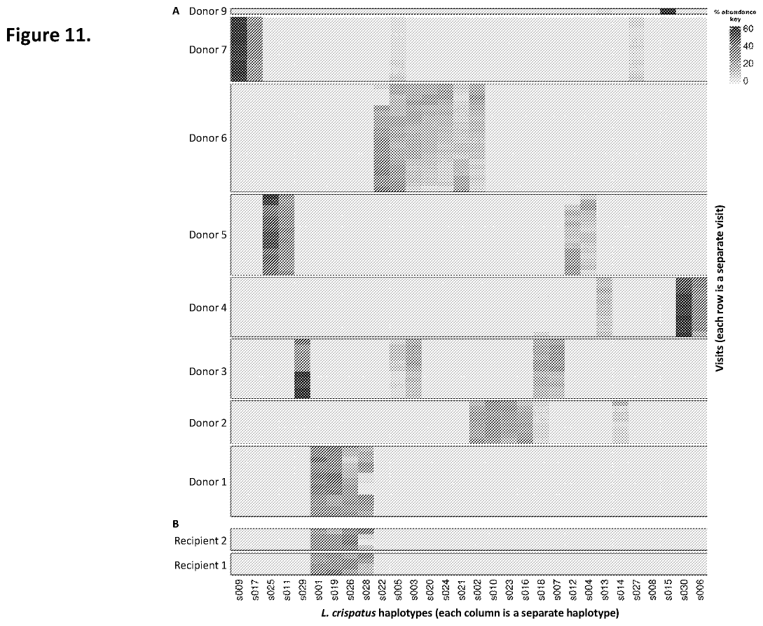

FIG. 11A shows an example of this for L. crispatus from donors 1-9. Each column represents one haplotype, wherein the darkness of the shading indicates relative abundance. Each row corresponds to a vaginal microbiota preparation obtained from a separate donor visit. Donor 8 is not shown at all and donor 9 only with one visit, as these donors were L. iners-dominant and did not have any L. crispatus. or only towards the end of their visits (FIGS. 6H-I).

In the example shown in FIG. 11A, this analysis showed 30 different L. crispatus haplotypes in total in the donor material. Some haplotypes were unique for one donor, while others were present in multiple donors. Some haplotypes consistently occurred together in the samples tested, indicating a possible symbiotic relationship. The data presented in FIG. 11A suggest that each donor had its own highly distinguishable haplotype signature comprising a plurality of L. crispatus haplotypes that were stable over multiple donations in any given donor but was distinct from the haplotype signatures of other donors. This information can be used to compare donor and recipient material and identify the lactobacilli strains that are able to successfully engraft, as is done in the next Example.

### Example 8

### Blinded in Silico Engraftment Analysis on Haplotype Level

This Example describes how the haplotypes generated in Example 7 can be used to determine engraftment of a SCVMP from a certain donor in its respective recipient.

To analyze engraftment by the specific donor's microbiome in the recipients' vaginal niche, the post-treatment recipient microbiome was compared to a library of all donor microbiomes (metagenomic sequencing data). The analysis was performed blinded, i.e., the analyst was not aware which donor the preparation was derived from. Recipient samples for the engraftment analysis were obtained at four different time points at 1 week, 1 menstrual cycle, and/or 2 menstrual cycles after SCVMP administration. In addition, samples from recipient 1 were obtained also 1 and 2 days after SCVMP administration.

Two recipients received SCVMPs from the same donor (Donor 1, but different substantially complete vaginal microbiota samples from separate visits). As can be seen from FIG. 11B, both recipient microbiomes have a haplotype signature (e.g., a set of lactobacilli haplotypes) that is identical to donor 1 and distinct from any of the other donors, suggesting that engraftment of the microbiome of donor 1 in the recipient was successful, and that the strains capable of engrafting into the vaginal niche of the recipient recapitulate the donor's microbiome. Only visits after SCVMP administration are shown, as the recipients did not have any L. crispatus prior to the administration (see FIGS. 9A and 9B for recipients 1 and 2, respectively) and no L. crispatus haplotypes were found in the pre-dosing visits in the recipients.

While currently available examples in literature compare transfer of therapeutic lactobacilli on a species level, the present analysis is more informative and conclusive, as it eliminates the possibility of the result being a naturally reverting microbiome, which is not detectable on a (higher) species level. The highly distinguishable haplotype signature of each donor (see FIG. 11A) allowed such analysis.

### Example 9

### Isolation of Bacterial Strains

This Example describes how single strains are isolated from the SCVMPs.

To start the isolation, the microbiota composition of the SCVMP was analyzed on both the species and the haplotype level. The isolation process was aimed at maximizing the number of isolated lactobacilli. For all Lactobacillus species except L. iners, MRS medium was used. For L. iners, MRS with cysteine was used. The minimum number of colonies to be picked was based on haplotypes found in the microbiome (see Example 8): for each major haplotype (present in high relative abundance), three colonies were picked, and for each minor haplotype (present in low relative abundance), five colonies were picked. This approach ensures that the number of colonies picked reflects the diversity in a preparation and hence maximizes the number of isolated lactobacilli. Generally, for each morphology 3 colonies were picked. If the number of morphologies was less than the number of haplotypes, more colonies were picked.

The starting point for isolation was a 50 μL aliquot of the SCVMP A dilution series of the SCVMP was plated on the selected medium (which was chosen based on the species present according to the sequencing analysis) and grown for 3 days at 37° C. in an anaerobic jar with gaspack. All plates were counted and closely observed to identify different colony morphologies. All work was performed under sterile conditions. Single colonies were described, picked and transferred to new plates of the same medium and grown under the same conditions until colonies were observed that can be picked (normally 1-3 days). Once grown on the second plate, colonies were again described and transferred to a third plate in the same way as with the second. If the colony morphology was not pure, a new lineage was started. After growth on the third plate single colonies were transferred to liquid medium of the same type as the plates. The liquid cultures were incubated at 37° C. in an anaerobic box with a gaspack. Once grown (normally 1-5 days), glycerol stocks were prepared, the pH was measured, and an aliquot was taken to be used for DNA extraction and sequencing.

The single isolate genomes were compared to the original metagenome of the donor's sample that the isolate was derived from. If this showed that not all of the metagenome of the original preparation was covered, more colonies were sent for sequencing.

### Example 10

### Selection and Characterization of Isolates for Clinical Development

This Example describes the selection and characterization procedure of how strains isolated from the SCVMPs are selected for clinical development.

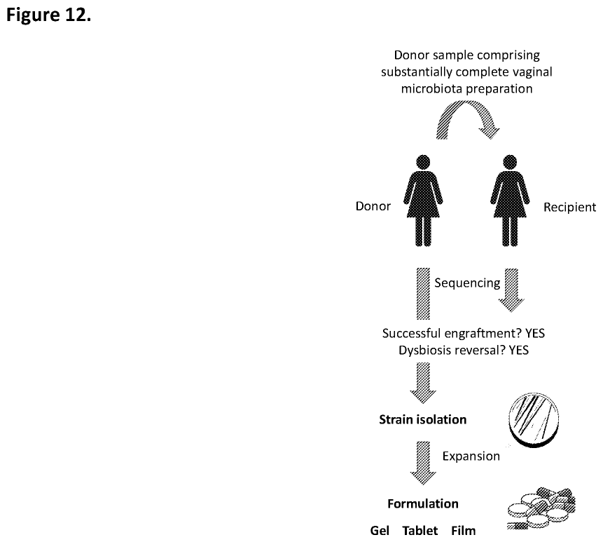

To select strain candidates for clinical use (hereafter simply referred to as ‘strain’ or ‘candidate strain’), a selection process is followed as outlined below. Step 1 is the key step in the selection process. One or more subsequent steps, in any combination, may be performed. This approach is different from previously described strain development programs, as it takes an in-human first approach and starts with a sample comprising strains that successfully engrafted in a recipient (“forward screen”). Strategies in the art typically start with a strain collection (with no prior knowledge of any strain collection member's ability to engraft from one person into another), wherein the collection comprises isolated strains which are tested in vitro regarding specific parameters, e.g., growth at low pH, followed by the transfer of selected isolated strain into a human subject and evaluation of engraftment (“reverse screen”). In contrast, the strategy described herein starts with strains that have shown clinical engraftment first (e.g., wherein a change of dysbiosis after transfer from a donor to a recipient as part of a SCVMP may be used as a readout), and wherein the strain(s) are isolated and identified second. This increases the chance of identifying strains from SCVMPs that are capable of engrafting and effecting a desired outcome (e.g., a change in vaginal dysbiosis and/or inflammation), compared to existing methods that attempt to determine engraftment chances of the isolated strains through in vitro approaches. A simplified schematic of the selection process described herein including formulations (described in Examples 11-14) is depicted in FIG. 12.

One or more of the following steps may be included in the selection process as desired:

Clinical engraftment: The first step and criterion for candidates is clinical engraftment of the strain(s) after administration of a donor's SCVMP to a recipient, optionally a recipient exhibiting dysbiosis. Suitable strain candidates engraft in at least one recipient and preferably more than one recipient, e.g., two, three or more different recipients.

Immune response: The immune response in a recipient after administration of the SCVMP (that the strain is derived from) is evaluated, using, for example, proteomics (e.g., Olink) and transcriptomics (e.g., Fluidigm or RNAseq) approaches. This can be done both on the level of the substantially complete preparation and also on the species-level using correlation matrices. Species/strains that result in upregulation of anti-inflammatory markers and/or downregulation of pro-inflammatory markers represent preferred potential candidate strains.

Safety profile: Safety can be evaluated at the level of the SCVMPs, e.g, as tested in human clinical trials. For example, in the event that an isolated strain originates from a SCVMP that showed product-related adverse events in a human trial, the isolated strain is non-preferred. A safety profile of the strains can additionally be assessed in silico and in vitro, by testing e.g., the presence of toxin genes and antibiotic resistance genes. Strains with a better safety profile (e.g., fewer resistances and/or toxin genes) are preferred.

Strain context: The strain context of the candidate strains is evaluated. As observed in Examples 5 and 6, SCVMPs differ in their complexity; wherein some may contain many species that all transfer to a recipient, other SCVMPs may contain only a few Lactobacillus species. As observed in Example 8, also on haplotype/strain level, there is variation in the number of strains present in a SCVMP. For creating a strain-based product for clinical use, one option is to combine all the strains originally present in the engrafting SCVMP. As consortia production is increasingly complex with larger numbers of strains/species in the product, SCVMPs with a simpler strain context (e.g., fewer total strains or species) are preferred over SCVMPs with a more complex strain context (e.g., many strains/species). Another option for creating a strain-based product for clinical use is to create a mixture of strains derived from one or multiple donors that together cover the full or a large part of a reference genome, e.g., a “pangenome” (and its functionality), in which case it is the broader genomic strain context that is used for selection rather than the single-SCVMP strain context. This approach is further described in point 6 in this Example.

Competition assay: In vitro competition assays can be performed with a recipient's or dysbiotic subject's CVS material against isolated strains and/or SCVMP derived from a donor. An example of a set up for an in vitro competition assay is a well-diffusion assay, where recipient/dysbiotic material is grown on an agar plate, and donor-derived material (e.g.,) or strains isolated therefrom are added to small wells punched into the plate. After several days of incubation, the plate is visually checked for clearing zones around the wells. It is thought that the larger the area where the (dysbiotic) recipient material has not grown around that well, the larger the inhibitory capacity of the donor-derived strain or preparation. Strains that produce large clearing zones (e.g., relative to other strains) and/or strains that show SCVMPs clearing zones with multiple different (dysbiotic) recipient materials are preferred. As a control for this assay, a donor SCVMP-recipient CVS pair is used that has been tested in the clinic.

In silico selection: In silico analysis can be performed on both functional and genomic/haplotype level, to identify genes and strains that are linked to, e.g. success of changing dysbiosis (e.g., a reversion from dysbiosis to a healthy vaginal microbiome, and/or a reduction of inflammation in the reproductive tract) in clinical trials conducted in humans with the SCVMPs from which the strains are derived. These data may, for example, reveal genes or metabolites that are required for engraftment success. Moreover, these data may be used to create a pangenome for each species, which provides the sequence of all genes of the engrafting strains of said species. The pangenome is the entire set of genes present in a selected set of organisms, in this case bacteria Strains can then be selected that cover (or a most homologous to) a preselected portion (or all) of the pangenome which can be combined into a strain-based product, e.g., to cover the desired pangenome expected to be involved in engraftment without using all strains originally present in the SCVMP.

In vitro and in silico characterization: In silico characterization of candidate strains can include genome assembly and functional annotation, as well as comparisons with other strains both from isolations from SCVMPs as well as public databases. In vitro assays on the candidate strains include, for example, determining D- and L-lactic acid production, acidification rate, pH optimum and pH tolerance, pathogen inhibition and survival, ability to culture and propagate, and detectability, stability, substrate utilization capacities, and requirements for, e.g., metals, vitamins, amino acids. The in vitro assay outcomes can be compared to engraftment data and engraftment pharmacodynamics. Strains that engraft in multiple recipients and/or engraft faster than others are preferred. Then, one or more in vitro assays can be consulted to further stratify the strains in terms of preferability, e.g., based on how the strains performed in these assays.

Pre-clinical in vitro model tests can be performed as further desired. These may include, for example, animal skin tolerance models and immune assays on cell lines.

### Example 11

### Multiparameter Assessment of Various Dosage Formulations Using Simulated Vaginal Fluid

A variety of suitable dosage forms were assessed for formulating the one or more bacterial strains identified by the methods of the invention, including formed gels, lyophilized gels, tablets, and films. A number of excipients were assessed to achieve suitable dosage forms, including mannitol, micro-crystalline cellulose, mucin (porcine, Sigma), hyaluronic acid (Sigma), maltodextrin, Guar gum (Sigma), inulin (Sigma), alginic acid (sodium alginate, Dupont), polyvinyl alcohol (PVA Parteck SRP 80, Merck), sodium CMC (Ac-Di-sol, sodium carboxymethyl cellulose, DuPont), polyvinylpyrrolidone (Kollidon (PVP), BASF), hydroxypropyl methylcellulose (Methocel K4M (HPMC), Colorcon), poloxamer (poloxamer 407 (Kolliphor), BASF), Carbopol (Carbopol 934, Serva), lactic acid, and acetate buffer.

### Basic Formulations:

Formed gels were prepared using the excipients including, e.g., hyaluronic acid, sodium alginate, HPMC/PVP, and poloxamer 407. The gels were pH adjusted to maintain a pH about pH 3.4-pH 3.9. A combination of lactic acid and acetate buffers were assessed.

Tablets were prepared using the excipients such as bulking agents including, e.g., microcrystalline cellulose, HPMC/PVP, maltodextran, and poloxamer 407 and compression. Lyophilized excipients were also evaluated to determine if muco-adhesion and/or gelling is improved. A combination of lactic acid and acetate buffer salts was assessed for inclusion. Tablet tensile strength was targeted at about 10 MPa, to ensure lack of excessive breakages and good processability. The target for pH of a reconstituted tablet (e.g., inside the vaginal cavity) was about pH 3.4-pH 3.9. The disintegration profile in a low liquid volume environment (e.g., inside the vaginal cavity) was also assessed.

For lyophilized gels, a number of excipients were lyophilized, including, e.g., hyaluronic acid, sodium alginate, HPMC/PVP, and poloxamer 407. All gels were pH adjusted to maintain a pH about pH 3.4-pH 3.9. A combination of lactic acid and acetate buffers were assessed. The target lyophilized appearance was a clean, uniform cake. The target water content was generally less than 3% w/w water (e.g., to increase viability of drug substance). The target reconstitution time in a vial was less than 2 minutes with hand swirling.

For films (air-dried) PVA in various concentrations was assessed. For example, PVA was supplemented with sodium CMC and other excipients to modify drying times, final flexibility of films, muco-adhesion etc. A combination of lactic acid and acetate buffers was assessed, and pH effect on immersion in simulated vaginal fluid evaluated. The target film properties included sufficient flexibility for application (e.g., to the vaginal tract), non-tackiness, acceptable drying times for ease of processing, a suitable film thickness, and disintegration profile in a low liquid volume environment (e.g., inside the vaginal cavity), as well as effective release and engrafting of drug substance. The target for pH of a reconstituted film (e.g., inside the vaginal cavity) was about pH 3.4-pH 3.9.

All components were assessed for optimization of viscosity (e.g., at 37° C., e.g., inside the vaginal cavity), enhancement of muco-adhesion (e.g., to epithelial, mucosal surfaces e.g., inside the vaginal cavity) and/or positive impact on drug substance stability (e.g., bacterial viability upon formulation and storage). Ideal formulations show little to no flow on suitable vertical surfaces and maintain high bacterial viability (e.g., CFU count) both upon formulation and during (long-term) storage.

Formulation selection parameters that were assessed included: Muco-adhesion (of reconstituted product, e.g., in the vaginal tract); viscosity (of reconstituted product), e.g., final viscosity for gel-based product needs to be syringeable at ambient temperature and preferably congealed at 37° C. (at body temperature, e.g., in the vaginal tract); total sugar content (of reconstituted product), e.g., ideally at or lower than physiological concentration (about 0.5-1.0 mg/mL); volume of reconstituted product, e.g., up to 3 mL; hydration rate/disintegration rate (e.g., of gel/matrix), e.g, sufficient physical integrity to provide desired release rate; pH, e.g, between about pH 3.4-3.9 (e.g., to promote inhibition of competitive vaginal bacteria); water activity/moisture content (e.g., of dried formulations), e.g., between 0.5-3% water (e.g., for longer term dried formulation stability); microbial diversity, e.g., relative abundance of Lactobacillus species, such as, e.g., L. crispatus, L. gasseri, L. jensenii, and L. iners; total dose/potency, e.g., preferably above 1×10⁵ CFU per administration (per dose), shelf-life (not reconstituted) at various temperatures, and microbial limits, e.g., absence of microorganisms such as, Pseudomonas aeruginosa, Candida albicans, Staphylococcus aureus, Ph Eur criteria 5.1.4, 2.6.12 & 2.6.13).

Testing was performed using standard assays, including plate count (e.g., MRS agar) or viable cell count (VCC, e.g., using Quantom Tx automated counting system (AM620) with fluorescent stain), e.g., for life bacteria count, dose determination, shelf-life; rheometer, e.g., for muco-adhesion and viscosity, pH meter, Karl Fisher/water activity meter, Ph Eur testing, e.g., for microbial loads.

### Gel Formulations:

Gel formulations were prepared with the following excipients:
Hyaluronic Acid at 0.5% and at 2%
HPMC+Carbopol+PVP
Poloxamer 20%+Sodium Alginate 2%
PVA 3%+NaCMC 2%
Poloxamer 20%+Guar gum 2%
Guar gum at 0.5% and 2%
Mucin 5%
Inulin 8%

Flow rates (viscosity, mPa×sec/shear rate 1/sec) under gravity at ambient temperature were determined with 0.5 mL on a 56 mm glass slide for Hyaluronic acid 0.5%, HPMC Kollidon 25+Carbopol, Guar gum 0.5%, Simulated mucus and inulin. Values obtained ranged from 300 to 1×10⁷ mPa×sec at shear rate of 0 to 3 to 7,000 mPa×sec at a shear rate of 100 1/sec, depending on formulation. Guar gum (0.5%) and hyaluronic acid (0.5%) did not appear to form gels, yielding low viscosities. Viscosity against temperature from 15 to 45° C. (with fixed shear rate of 10 s-1) was also determined. Poloxamer showed a thermo-reversible gelling behavior with an increase in viscosity at about 25° C. (from about 10,000 to about 12,000 mPa×sec, while other formulations mostly showed small changes in their viscosities at various levels between about 500 and 12,000 mPa×sec at 15° C. and about 200 to 11,000 at 45° C., depending on formulation.

The next step included an assessment which of the gel bases could be lyophilized and reconstituted to form an acceptable gel. The appearance of a ‘cake’ was also evaluated to indicate homogeneity.

A simulated vaginal fluid (SVF) was produced, with the following composition: 35 mg of NaCl, 14 mg of KOH, 22 mg of calcium hydroxide, 20 mg of lactic acid, 10 mg of acetic acid, 1.6 mg of glycerol, 50 mg of glucose into 10 mL of water, adjusting to pH 4.2 using HCL.

After suitable excipients were determined, these excipients were used to form gels by adding 1.5 mL of SVF. The addition of SVF allowed correct pH balance and simulate the addition of a substantially complete vaginal microbial preparation to the excipient base. Samples were hand-shaken within vials to form homogenous gels and then lyophilized. Some excipients produced acceptable appearance post-lyophilization. PVA, Guar gum and HA, formed acceptable (clear) gels; Kolliphor P 407 (Poloxamer 407) and Methocel K4M formed white, crystalline cakes; maltodextrin and PVP displayed discolorations (e.g., yellow), whereas sodium alginate, mucin, and NaCMC produced non-uniform cakes with discolorations (e.g., brown). Surprisingly, mannitol which typically forms consistently acceptable cakes, was crystalline which is probably due to the salt contents in the SVF.

The resulting gels were reconstituted with 1.5 mL deionized water post lyophilization. HA, guar gum and PVA reconstituted easily, with reconstitution times of 2:30 min, 2:30 min, and 1:30 min, respectively. HA formed a homogenous gel with a pH of 4.6 Guar gum formed a non-homogenous gel with a pH of 4.5. PVA formed a homogenous liquid with some foaming with a pH of 4.7. PVP, NaCMC, poloxamer, sodium alginate and HPMC formed semi-homogenous gels. All required more than 3 minutes for reconstitution. PVP dissolved slowly with a pH of 4.1. NaCMC formed a non-homogenous viscous liquid with a pH of 4.9. Poloxamer displayed some foaming with a pH of 5.4. HPMC formed a gel with some foaming with a pH of 4.4. Sodium alginate was non-homogenous with a pH of 5.5. Mannitol formed a homogenous liquid with some crystals remaining with a pH of 4.4.

A muco-adhesion test was conducted to study the effect under gravity of the drug product when applied. For this, a mucosal surface (such as would be present in the vaginal cavity) was simulated. 500 mg of mucin was compressed into disc a with a flat 2 cm tooling to approximately 3 tons of pressure. These were then adhered to a substrate, e.g., the bottom of a plastic box. Each disc was then wetted with approximately 200 μL of deionized water and rubbed with a gloved finger until the surface of the mucin became tacky to the touch. About 0.5 mL of sample (or the complete dosage unit, in the case of tablets and PVA films) was then applied in the horizontal position and allowed to settle for 2 minutes. The box was then raised so that samples were in a vertical position, and a visual assessment was taken with respect to adherence properties (muco-adhesion test) of the Guar gum, poloxamer, mannitol, PVP, hyaluronic acid, sodium alginate, CMC, HPMC, and PVA gel samples. Hyaluronic acid, NaCMC, sodium alginate and HPMC showed the best muco-adhesion in this test.

As poloxamer showed thermo-reversible behavior in earlier tests, the muco-adhesion testing was repeated on a pre-heated plastic box and mucin disc at 37° C. It was confirmed that the gel that formed was more viscous than at ambient temperature and retained on the surface of the wetted mucin disc. This indicates that the poloxamer may be at suitable viscosity at body-temperature

Syringeability was also assessed following adjustment of concentrations: 3% (45 mg) carbopol, 4% (60 mg) sodium alginate, 3% (45 mg) HPMC & 1.9% (30 mg) sodium CMC, and 24% (360 mg) poloxamer. These were prepared using an SVF: lactate buffer mix. The inclusion of a lactate buffer in the pH range of ˜3.4-4 may promote engraftment of the lactic acid bacteria in vivo (e.g., in the vaginal tract), e.g., potentially by minimizing the competition of undesirable bacterial taxa resident in the existing microbial niche. Excipient were mixed with 1.5 ml SVF+lactate buffer. Carbopol and poloxamer both formed homogenous gels after hand shaking with some air bubbles which dispersed when settled. Sodium alginate and HPMC+NaCMC did not form homogenous gels.

A lyophilized gel format as a dosage form for the one or more bacterial strains identified by the methods of the invention or the substantially complete vaginal microbial preparation can, for example, be produced by blending with excipients followed by lyophilization and packaging, e.g., in vials. The vials can then be reconstituted, e.g., in a clinic setting, with water to form a gel in the vial prior to application of the reconstituted drug product, e.g., by using an applicator, such as a syringe, to administer the composition comprising the one or more bacterial strains identified by the methods of the invention or the substantially complete vaginal microbial preparation to the vaginal tract of a subject.

A frozen gel describes a dosage form for the one or more bacterial strains identified by the methods of the invention or the substantially complete vaginal microbial preparation that is blended with gelling excipients (optionally along with a suitable lactate buffer) and the liquid gel form is then frozen (at −80° C.) and stored in either a vial or a pre-filled syringe.

### Tablet Evaluation:

In addition to gels, tablet-pessaries were generated as an additional dosage form. The following aspects were considered: choice of excipient suitable for compression to form tablets and for lyophilization, as well as to provide acceptable level of muco-adhesion, optional inclusion of lactate buffer with a pH target of about pH 3.5-4, optionally with the aim to have a single tablet prepared from individual cervico-vaginal fluid donations. All of the assessed excipients which produced both acceptable gel formation and acceptable lyophilization (e.g., to form a reasonably free-flowing powder for further processing) were evaluated (alongside additional excipients) for tableting quality on a manual tablet press, including Carbopol, HPMC, gelatin, sodium alginate, poloxamer, NaCMC, and pectin. Tablets were compressed at about 1 ton a with 5.5 mm tooling to form tablets. Tablets were gently added to wetted mucin discs and muco-adhesion assessed, as described earlier.

All tablets remained attached for over 5 minutes. Tablets were rinsed every minute to assess impact of adhesion. NaCMC began to swell into a gel. Tablets of carbopol, HPMC, alginate, NaCMC showed sufficient tablet integrity. Poloxamer formed a tablet but with relatively low strength. Pectin and gelatin tablets showed low hardness and some brittleness.

### Polymeric Films:

Film forming/casting is a potential route of preserving viability in live biological materials. To investigate this, the following excipient test samples were generated;
PVA 3%
PVA 20%
PVA 3%, NaCMC 2%
PVA 20%, NaCMC 5%

All samples were mixed as necessary to make a gel/film and transfer into plastic blister pack (size 0) and were stored at ambient conditions for about 48 hours to set.

Both samples containing 3% PVA did not form suitable films. Films containing NaCMC displayed brittleness and were difficult to remove from the blister mold. The 20% PVA sample did form a film and could be removed with ease from the blister mold. The film was flexible and resistant to tearing.

To evaluate whether forming a film with the inclusion of the one or more bacterial strains identified by the methods of the invention, or the substantially complete vaginal microbial preparation is feasible, a ‘sandwich’ approach to film preparation was tried. This was performed in an upturned Karl Fischer lid (with PTFE lining), to produce a mold with a flat disc design. A 12% PVA solution (from the previous 20%) was used to reduce the initial viscosity and allow more consistent sample preparation via pipette. Preparation was performed as follows:

0.25 mL of 12% PVA was pipetted into each mold, followed by 0.5 mL of SVF. Another 0.25 mL of 12% PVA was pipetted on top to complete the ‘sandwich’. The molds were left to solidify.

After about 48 hours, discs were formed, which were flexible and left no apparent residue on the PTFE lining. This suggests that the PTFE may be a suitable material for molding, the film was easily recovered from this surface.

Films were gently added to mucin discs and muco-adhesion assessed, as described earlier. The PVA discs showed no movement from the mucin surface, indicating good muco-adhesion. A wash of about 2 mL of SVF was performed after 5 minutes, which did not displace the PVA discs from the mucin surface.

TABLE 5
Formulation summary:

| | | | Lyophilized/ | |
|---|---|---|---|---|
| Chemical | | | frozen liquid | Film-form |
| name | Functionality | Tablet | gel | (air-dried) |
| Hyaluronic acid | Structuring polymer | ++ | + | − |
| Sodium alginate | Structuring polymer | − | − | − |
| Maltodextran | Bulking agent, carbon | − | − | − |
| | source | | | |
| Guar gum | Bulking agent, prebiotic/ | + | + | − |
| | viscosity agent | | | |
| NaCMC + HPMC | Polymer combination | + | + | − |
| Polyvinyl alcohol | Structuring polymer | − | − | ++ |
| Sodium CMC | Polymer/mucoadhesive | ++ | ++ | − |
| Polyvinylpyrrolidone | Structuring polymer | ++ | + | − |
| Hydroxypropyl | Structuring polymer | ++ | ++ | − |
| methylcellulose | | | | |
| (HPMC) | | | | |
| Poloxamer 407 | Structuring polymer | − | ++ | − |
| Carbopol 934 | Polymer/viscosity modifier | ++ | ++ | − |
| Mannitol | Control | − | − | − |
| Sodium lactate | pH modification | ++ | ++ | ++ |
| Lactic acid | pH modification/ | ++ | ++ | ++ |
| | enhancement of drug | | | |
| | product effectiveness | | | |

++ suitable for dosage form
+ potentially suitable for dosage form
not suitable for dosage form

### Example 12

Assessment of Bacterial Viability when Frozen with Cryoprotectant

Alongside the development of a dosage form, such as a gel, tablet of film, the stability of an L. crispatus strain isolated from donor 1 was evaluated in a liquid format at −20° C. and −80° C. This format can be used as an intermediate product which may then be combined with, e.g., gelling agents in clinic (e.g., prior to administration to the vaginal tract) to form a suitable drug product. The surrogate taxon simulates an isolated Lactobacillus strain identified by the methods of the invention.

Two concentrations of the cryoprotectant glycerol were evaluated along with two concentrations of mucin. The pH of the medium was also investigated using a phosphate buffered saline, and 1% lactate buffer at pH 3.4-4:

An L. crispatus stock was prepared from a bacterial isolate as described herein, incubated in MRS broth at 40° C. for approximately 5 days. The L. crispatus stock was diluted with the below formulations in Table 6 to an appropriate concentration (1×10⁸ CFU/mL).

TABLE 6
Testing cryoprotectants

| Control/ | Final | Lactate |
|---|---|---|
| cryoprotectant | concentration | buffer |
| PBS, control | — | — |
| PBS, control | — | 1% |
| Glycerol | 10% | — |
| Glycerol | 25% | — |
| Glycerol | 10% | 1% |
| Glycerol | 25% | 1% |
| Mucin | 2% | — |
| Mucin | 5% | — |
| Mucin | 2% | 1% |
| Mucin | 5% | 1% |

A freeze/thaw study was conducted that consisted of: Samples frozen (1 day, −80° C. and −20° C., respectively), defrosted and tested (subsampled-1 day), frozen a second time (1 day, −80° C. and −20° C., respectively), and defrosted and tested (1 day), frozen a third time (1 day, −80° C. and −20° C., respectively), and defrosted and tested (1 day). Viable cell count was assessed. The higher glycerol concentrations offered the best cryoprotection with increased viability at −80° C. storage. Samples at −20° C. showed low viability after the third freeze/thaw cycle. After 1 cycle, on average, at −20° C. the samples were between 4×10⁶ viable cells/mL and 2×10⁷ viable cells/mL, whereas the samples at −80° C. were 4×10⁶ viable cells/mL and 6×10⁷ viable cells/mL. The viability didn't change significantly between 1^{st} and 3^{rd} cycle with a decrease of 0-1 log for storage at −20° C. and no further decrease at −80° C.

A stability study was conducted that consisted of: VCC prior to freezing, at 1 month and 2 months in frozen storage. Stability data at −20° C. and −80° C. gave similar results to the freeze-thaw study, in that the −20° C. storage showed a larger loss of viability of L. crispatus than what is seen for storage −80° C. Higher glycerol content increased cryoprotection and viability was maintained better in −80° C. with only about 0.5 log drop over 2 months. For purposes of storage, there was very little difference between 1% lactate buffer and PBS medium. The final glycerol concentration will likely need to be adjusted to lower than 25%, e.g., 20%, 17.5%, 15%, 12.5% or 10% as high glycerol concentrations may undesirably affect the physical characteristics of certain formulations/dosage forms, such as, e.g., gels and tablets.

### Example 13

Assessment of Bacterial Viability when Lyophilized with Various Excipients

Suitable excipients were selected on the basis of their physical properties, such as the quality of cake formation, ease of reconstitution following lyophilization and homogeneity of the resulting gel (see, previous examples). Next, it was assessed which excipients provide most viability protection during the lyophilization and manufacturing process.

To evaluate this, individual excipients were combined with L. crispatus to simulate a substantially complete vaginal microbial preparation. The L. crispatus stock was diluted with the formulation to an appropriate concentration (1×10⁸ CFU/mL). Mannitol and maltodextran were included as control samples, as they typically provide adequate viability protection during lyophilisation.

TABLE 8
Formulations for viability testing upon lyophilization.

| | | | % w/v after | |
|---|---|---|---|---|
| Formulation | Chemical | % w/v in | 1.5 mL | VCC/mL after |
| No | name | buffer stock | reconstitution | lyophilization |
| 1 | Hyaluronic acid | 4.5 | 3 | 1.22E+08 |
| 2 | Maltodextran | 7.5 | 5 | 1.34E+08 |
| 3 | Guar gum | 4.5 | 3 | 1.44E+08 |
| 4 | Polyvinyl alcohol | 15 | 10 | 1.33E+08 |
| 5 | Sodium CMC | 12 | 8 | 2.02E+08 |
| 6 | Polyvinylpyrrolidone | 15 | 10 | 2.13E+08 |
| 7 | Hydroxypropyl | 18 | 12 | 4.30E+07 |
| | methylcellulose | | | |
| 8 | Poloxamer 407 | 30 | 20 | 2.54E+08 |
| 9 | Carbopol 934 | 4.5 | 3 | 7.05E+07 |
| 10 | Mannitol | 7.5 | 5 | 2.69E+08 |
| Buffer for | Sodium lactate | 0.75 | 0.5 | — |
| make up | Lactic acid | 0.75 | 0.5 | — |

A reasonable viability retention (% VCC recovered from stock) was obtained for the majority of excipients. As expected, mannitol performed well as lyophilization protectant, and served as a control for the process. HPMC, showed the lowest recovery of viability.

Viability recovery for carbopol as an excipient was also lower compared to other excipients but it displayed desirable gel characteristics in earlier experiments. It should be noted that the lyophilized material can be filled into capsules which represent another suitable dosage form for vaginal administration of a composition comprising a substantially complete vaginal microbiota preparation, though capsule filling was not tested.

The most preferred excipients for tablets are sodium CMC, polyvinylpyrrolidone and poloxamer. Sodium CMC tableted well and was also able to form a homogeneous suspension upon reconstitution. Polyvinylpyrrolidone lyophilized well, is suitable for tablet compression and gave good viability. Poloxamer provided good viability retention and can be optimized for use in tablet formulation.

The most preferred excipients for lyophilized gels are hyaluronic acid, Guar gum, and sodium CMC. Hyaluronic acid exhibits desirable viscosity, the ability to resuspend homogeneously, and acceptable viability retention.

The most preferred excipients for frozen liquid gels are hyaluronic acid, poloxamer, and carbopol. Carbopol displays very good viscosity and homogeneity though lower viability retention is observed.

The most preferred excipient for (air-dried) films is polyvinyl alcohol which exhibited good viability retention.

### Example 14

### Assessment of Stability of Various Dosage Forms

This Example assesses the stability of various dosage forms.

The following dosage forms were generated for stability testing:
3 different gel formulations were generated based on Carbopol (Carbopol 934), hyaluronic acid, and poloxamer (poloxamer 407), respectively, which also contained sodium lactate, lactic acid and L. crispatus in simulated vaginal fluid. The gel formulations were placed at −80° C. directly following preparation.
3 different tablet formulations were generated based on polyvinylpyrrolidone, sodium carboxymethlcellulose, and poloxamer which also contained lactate buffer and L. crispatus in simulated vaginal fluid. The formulations were lyophilized and tabletted. The tablet formulations were stored at 2-8° C. and 25° C., respectively.
3 different lyophilized gel formulations were generated based on sodium carboxymethlcellulose, hyaluronic acid, and Guar gum, respectively, which also contained sodium lactate, lactic acid and L. crispatus in simulated vaginal fluid. The gel formulations were lyophilized and the resulting dry powder stored at 2-8° C. and −20° C., respectively.
1 film formulation containing PVA and L. crispatus in simulated vaginal fluid sandwiched between two layers of PVA (described earlier) was also evaluated. The film formulation was stored at 2-8° C.

The samples were tested for viable cell count (VCC) at t=0 right after formulation. Samples were reconstituted with PBS and adjusted to pH 7 to ensure the activity of the fluorescent dye used for VVC. The volumes of the sample preparations differed based on the final sample viscosities, and these volumes are compensated for in dilution factors in the results. Samples were also tested after 1 month (t=1) and 2 months (t=2).

TABLE 9
Formulation stability (VCC of different formulations at various temperatures)

| Formulation | Mean | t = 1 | t = 1 | t = 1 | t = 1 | t = 2 | t = 2 | t = 2 | t = 2 |
|---|---|---|---|---|---|---|---|---|---|
| Sample | t = 0 | (2-8° C.) | (−80° C.) | (−20° C.) | (+25° C.) | (2-8° C.) | (−80° C.) | (−20° C.) | (+25° C.) |
| PVA | 5.04E+08 | 7.04E+08 | | | | 5.57E+08 | | | |
| Form Gel polaxamer | 3.51E+07 | | | | | | 6.82E+07 | | |
| Form Gel HA | 1.66E+08 | 2.32E+08 | 2.01E+08 | | | | 2.97E+08 | | |
| Form Gel Carbopol | 4.42E+07 | | 5.65E+07 | | | | | | |
| Lyo Gel NACMC | 3.36E+08 | 4.80E+08 | | 4.25E+08 | | 2.91E+08 | | 4.63E+08 | |
| Lyo Gel HA | 5.35E+07 | 2.32E+08 | | 2.01E+08 | | 1.74E+08 | | 1.89E+08 | |
| Lyo Gel Guar Gum | 8.02E+06 | 6.97E+06 | | 1.84E+08 | | 2.73E+08 | | 5.00E+08 | |
| Tablet NACMC (3 tons) | 5.79E+08 | 3.01E+08 | | | | 4.37E+08 | | | |
| Tablet NACMC (1 ton) | 5.79E+08 | | | | 2.18E+08 | | | | 1.88E+08 |
| Tablet PVP | 1.19E+08 | 3.28E+08 | | | 2.88E+08 | 3.88E+08 | | | 1.92E+08 |
| Tablet Polaxamer | 2.60E+08 | 3.57E+07 | | | | 1.82E+07 | | | 2.81E+07 |

For the 2-8° C. storage conditions, PVA, lyophilized gel and tabletted NaCMC, tabletted PVP and lyophilized gel HA all appeared to retain substantial viability after 2 months. A decrease was observed in the viability in the poloxamer tablet already after 1 month.

Although the lyophilized guar gum had a lower initial viability, this was maintained at both tested timepoints. For the −80° C. storage conditions, the formed gels of both the Carbopol and the HA appear to have maintained the viability at a similar level to t=0 at the 1 or 2-month time point, respectively.

The viability measured for the formed gel of poloxamer was stable at the 2-month time point. The lyophilized gels for all formulations exhibited no significant loss of viability at 2 months. For the 2-8° C. storage conditions after 2 months, PVA yielded the highest viable cell count with a small margin, followed by NACMC.

For the tablet formulations stored at 25° C., NaCMC and PVP formulations showed no appreciable loss of viability.

Hyaluronic acid: In both the lyophilized gel (2-8° C. and −20° C.) and frozen formulations (−80° C.) yielded good viability protection during processing (T=0) and on stability at all studied temperatures.

Sodium CMC: In both the lyophilized gel (2-8° C. and −20° C.) and tablet forms (2-8° C. and 25° C.) yielded good viability protection both during processing (T=0) and on stability at all studied temperatures.

Polyvinylpyrrolidone: In the tablet form (2-8° C. and 25° C.) yielded good viability protection both during processing (T=0) and on stability.

Polyvinyl alcohol: As an air-dried film disc (2-8° C.) retained viability, yielding higher VCC than all other tested formulations at 1 and 2 months.

Guar gum: The lyophilized gel (2-8° C. and −20° C.) showed good viability after storage.

Carbopol: In the frozen liquid gel (−80° C.) showed an initially lower viability in comparison to both alternative frozen liquid gels but appeared to maintain this level at t=1 month.

### Example 15

### In-Human Clinical Testing

This Example describes in-human tests of the selected strains isolated from substantially complete vaginal microbiota preparations.

A plurality of isolated multiple strains and single strains (e.g., identified as described in Examples 6-9, and isolated as described in Examples 10 and 11) that pass through pre-clinical trials with a satisfactory safety profile are tested to determine in-human safety and efficacy in volunteer subjects with or without vaginal dysbiosis. The subjects can be symptomatic or asymptomatic for vaginal health symptoms. In a placebo-controlled manner, participants are dosed daily for up to a month with vaginally applied preparations of multiple strains (strain consortia) and/or single strains, optionally in a variety of formulations, e.g., such as described in Examples 11-14. In multiple arms of the clinical study, different doses over different periods of time (up to one month) are tested, e.g., with daily self-administered applications. In some embodiments, the first dose is also applied with or without an antiseptic vaginal pretreatment, wherein the pretreatment may be performed by an investigator. Outcomes from these in-human trials are conducted to determine the effect of the isolated strains or strain consortia on changes in endometrial microbiome, vaginal microbiome, local (pro- and anti-inflammatory) biomarkers in vaginal and/or endometrial tract, and other markers, analytes and readouts related to controlling inflammation and longer-term colonization of the strain or strain consortia during and post daily dosing. The effects are monitored for several weeks up to several months post dosing. The changes of the endometrial microbiome or biomarkers of the endometrial tract are analyzed from a sample of aspirated endometrial fluid.

To determine the safety profiles of the interventions, frequency and severity of adverse events are assessed, including colposcopy findings at days 7 and 14 of dosing and day 7 post-dosing, chemistry/hematology routine safety laboratories and acceptance based on self-administered questionnaires.

The in-human clinical testing also involves testing the resilience of vaginal colonization of the isolated strains and strain consortia upon hormonal changes in the subjects (either through natural fluctuation, e.g., menstrual cycle or hormone administration, e.g., estrogen and progesterone).

Alternatively, or in addition, a two-step clinical study may be conducted, wherein first a strain consortium comprising 8 to 10 bacterial strains is administered and assessed for the strains capability to engraft and for immune changes. In a subsequent second step, a subpopulation (or one or more single strains) of those 8-10 strains that were administered in the first step, are selected based on high levels of immune modulation and engraftment.

1. A method of identifying one or more bacterial strains capable of colonizing a female genitourinary tract comprising:
providing a donor sample comprising a substantially complete vaginal microbiota preparation (SCVMP) derived from a donor female comprising a plurality of bacterial strains;
administering an effective amount of the donor sample to a recipient female's genitourinary tract that exhibits a dysbiotic vaginal microbial niche;
assessing a desired change in dysbiosis of the recipient female's genitourinary tract over a predetermined time period;
identifying one or more bacterial strains of the donor sample that colonize the vaginal microbial niche of the recipient female's genitourinary tract by analyzing a recipient sample comprising a plurality of bacterial strains obtained from the recipient female's genitourinary tract after achieving the desired change in dysbiosis.

2. The method of claim 1, further comprising (a) nucleic acid sequencing of the microbial constituents of the recipient sample, and of the donor sample, (b) comparing the sequencing results for the microbial constituents of the recipient sample with the microbial constituents of the donor sample to determine sequence identity of strains residing in both, and (c) determining that one or more strains is capable of colonization if the one or more strains identified from the recipient sample match one or more strains from the donor sample by a predetermined degree of sequence identity or by their haplotype signatures.

3. The method of claim 2, wherein said nucleic acid sequencing is whole genome sequencing or haplotype analysis.

4. The method of claim 2, wherein said predetermined degree of sequence identity in a defined genetic region between one or more strains of the donor sample and the one or more strains of the recipient sample is at least 98%, 99%, 99.5%, or 99.9%.

5. The method of claim 1, wherein one or more strains of genus Lactobacillus is identified as capable of colonizing.

6. The method of claim 5, wherein the one or more strains of the genus Lactobacillus is identified (e.g., detectable by sequencing) in the donor sample and in the recipient sample after administration of the donor sample to the recipient female but not prior thereto.

7. The method of claim 5, wherein the one or more strains of the genus Lactobacillus is identified (e.g., detectable by sequencing) in the recipient sample after a desired change in dysbiosis of the recipient female's genitourinary tract has occurred.

8. The method of claim 5, wherein the one or more strains of the genus Lactobacillus is identified (e.g., detectable by sequencing) in the recipient sample after one (two, three, four or five) menstruation cycle(s) post administration of the donor sample.

9. The method of claim 5, wherein the one or more strains of the genus Lactobacillus is identified (e.g., detectable by sequencing) in the recipient sample after one (two, three, four, five, six, or 12) month(s) post administration of the donor sample.

10. The method of claim 1, wherein the substantially complete vaginal microbiota preparation (SCVMP) is obtainable by a method comprising
A. providing a microbiota sample from a donor female genitourinary tract; wherein step A comprises one, two, or three of steps (1), (2), (3) or any combination thereof, and both steps (4) and (5):
(1) adding a diluent to the microbiota sample to create a diluted sample,
(2) removing a portion of the diluted microbiota sample for testing (e.g., nucleic acid sequencing),
(3) pre-cooling for either refrigeration or freezing of the remainder of the microbiota sample,
(4) storing the refrigerated or frozen microbiota sample under quarantine,
(5) holding the refrigerated or frozen microbiota sample under quarantine until any completion of any combination of (a) testing the donor to exclude the substantial presence of one or more transmissible pathogens (e.g., blood, and/or cervicovaginal secretions, and/or urine sample testing), (b) confirming the composition and viability of the microbiota, or (c) further confirming the health of the female donor by a plurality of post-screening tests occurring within a time period of 30-90 days post-donation; and
B. releasing the refrigerated or frozen microbiota sample from quarantine to define the microbial constituents of the SCVMP, e.g. by nucleic acid sequencing.

11. The method of claim 10, further comprising a step of
pooling the sample from the same donor (or from different donors) to obtain a sample of predetermined quantity (e.g., of CFUs contained in the sample), and/or
filtering the sample (e.g., to remove cell or bacterial aggregates or other unwanted matter residing in the cervicovaginal secretion of the donor sample).

12. The method of claim 10, wherein the substantially complete vaginal microbiota preparation comprises
(i) one, two, three or four bacterial species from the genus Lactobacillus, selected from Lactobacillus crispatus, Lactobacillus iners, Lactobacillus jensenii, Lactobacillus gasseri, which comprise >80-99.9% of all detectable bacterial species of the preparation; and
(ii) less than 5% of Gardnerella spp., Atopobium spp., and Prevotella spp.

13. The method of claim 10, wherein the substantially complete vaginal microbiota preparation further comprises at least one further Lactobacillus selected from the group consisting of L. acetotolerans, L. acidophilus, L. amylovorus, L. gallinarum, L. gigeriorum, L. helveticus, L. johnsonii, L. kefiranofaciens, L. kitasatonis, L. paragasseri, L. psittaci, Lactobacillus sp002911475, L. taiwanensis, L. ultunensis, L. coleohominis, L. reuteri, and L. vaginalis.

14-22. (canceled)

23. The method of claim 1, wherein the desired change in dysbiosis of the recipient female's genitourinary tract is detectable within two, three, six, nine, or twelve months post administration of the donor sample.

24. The method of claim 1, wherein the desired change is a drop in vaginal pH, e.g., by at least pH 0.3, 0.5, 1.0 or at least pH 1.5.

25-37. (canceled)

38. The method of claim 1, further comprising determining that the donor sample is substantially free of Gardnerella spp., Atopobium spp., and Prevotella spp. prior to administration to the recipient, optionally, wherein the preparation comprises less than 5% of Gardnerella spp., Atopobium spp., and Prevotella spp.

39. (canceled)

40. The method of claim 1, further comprising determining that the donor sample is substantially free of human sperm (spermatozoa) prior to administration to the recipient.

41. The method of claim 1, further comprising determining that the female donor is substantially free of any one or more (two or more, three or more, or four or more) of: (i) bacteria involved in bacterial vaginosis (e.g., Gardnerella spp., Prevotella spp. and Mobiluncus spp.), (ii) yeast (e.g., Candida, Cryptococcus, and Saccharomyces species), (iii) sexually transmitted pathogens (including Neisseria gonorrhea, Chlamydia trachomatis, and Trichomonas vaginalis), (iv) bacteria involved in urinary tract infections (e.g., Escherichia. coli, Staphylococcus, Chlamydia, and Mycoplasma), and (v) viruses (e.g., HIV, human papilloma virus (HPV), hepatitis B virus, hepatitis C virus, HSV-2).

42. (canceled)

43. The method of claim 1, wherein the donor sample comprises about 80-99.9%;
(a) Lactobacillus crispatus;
(b) Lactobacillus iners;
(c) Lactobacillus jensenii; and
(d) Lactobacillus gasseri.

44-63. (canceled)

64. A method of defining one or more vaginal microbial donor communities, the method comprising:
obtaining a plurality of vaginal donor samples, wherein each donor sample comprises vaginal microbes obtained from a different non-dysbiotic (e.g., healthy) donor female (e.g., from vaginal fluid, e.g., a cervicovaginal secretion),
using an analytical technique to obtain a plurality of vaginal microbial profiles from the plurality of vaginal samples,
identifying a profile based on the plurality of microbial profiles, thereby defining a plurality of vaginal microbial donor communities,
wherein the profile is selected at least in part based on identifying at least one genetic element (or a plurality of genetic elements) associated with the capability to successfully engraft/colonize the female genitourinary tract of a female recipient, and
providing the profile to a user.

65. The method of claim 64, wherein the microbial profiles are obtained by preparing at least one nucleic acid sample from at least one species of microbiota present in the vaginal donor sample.

66-71. (canceled)

72. The method of claim 64, wherein the user obtains the profile for the purpose of determining a suitable donor for administration of the vaginal microbial donor communities to a recipient in need thereof.

73. (canceled)

74. A composition or pharmaceutical composition comprising one or more (isolated) bacterial strains capable of colonizing and engrafting the vaginal microbial niche identified according to the method of claim 1.

75. A composition or pharmaceutical composition comprising one or more (isolated) bacterial strains which form a vaginal microbial donor community as defined according to claim 64.
